(19) United States
(12) Patent Application Publication
(10) Pub. No.: US 2018/0200403 A1
(43) Pub. Date: Jul. 19, 2018

(54) FILM, MANUFACTURING METHOD THEREOF, AND APPLICATION THEREOF

(71) Applicant: Industrial Technology Research Institute, Hsinchu (TW)

(72) Inventors: Wei-Hong CHANG, Linnei Township (TW); Ming-Chia YANG, Taipei City (TW); Yun-Han LIN, Taichung City (TW); Kun-Mao KUO, Tainan City (TW); Yu-Chi WANG, New Taipei City (TW); Hsin-Hsin SHEN, Zhudong Township (TW); Li-Jie LIN, New Taipei City (TW)

(73) Assignee: Industrial Technology Research Institute, Hsinchu (TW)

(21) Appl. No.: 15/850,902

(22) Filed: Dec. 21, 2017

Related U.S. Application Data
(63) Continuation-in-part of application No. 15/388,980, filed on Dec. 22, 2016.

(30) Foreign Application Priority Data
Dec. 21, 2016 (TW) 105142419

(51) Int. Cl. A61L 24/04; A61L 24/00; A61B 17/06

(52) U.S. Cl. CPC A61L 24/043; A61B 2017/00942; A61B 17/06166; A61L 24/0094

(57) ABSTRACT

The present disclosure provides a film composed of a polymer mixture, wherein the polymer mixture includes: a hydrophobic composition including polycaprolactone (PCL); and at least one hydrophilic polymer selected from a group consisting of alginate, gelatin, hyaluronic acid, polyvinyl alcohol (PVA), carboxymethyl cellulose (CMC), polyethylene glycol (PEG), collagen, demineralized bone matrix (DBM), bone morphogenetic protein (BMP), albumin, chitosan, fibrin, polyoxyethylene, polyvinylpyrrolidone, wherein the weight ratio of the hydrophobic composition to the hydrophilic polymer is about 1:0.01-100, and wherein the film has the effect of preventing leakage from a surgical wound or a diffuse wound.

### CROSS REFERENCE TO RELATED APPLICATION

This application is a Continuation-In-Part of application Ser. No. 15/388,980, filed on Dec. 22, 2016, which is based on, and claims priority from, Taiwan Application Serial Number 105142419, filed on Dec. 21, 2016, the disclosure of which is hereby incorporated by reference herein in its entirety.

### TECHNICAL FIELD

The technical field relates to a film, a manufacturing method thereof, and the use thereof.

### BACKGROUND

Tissue adhesives and sealants have many potential medical applications, such as wound closures; as a supplement or substitute for sutures or staples used in surgical operations; to prevent the leakage of fluids such as blood, bile, gastrointestinal fluids and cerebrospinal fluid; or for affixing a surgical mesh to soft tissue. Fibrin is the most widely used of such adhesives. However, fibrin has defects that include slow solidification and poor mechanical strength. Moreover, when using fibrin, there is a risk of viral infection, and tissue adhesion can easily occur on the wound. Thus the use of fibrin is limited in surgical applications.

In cardiovascular surgery or a liver, gallbladder, intestine, or stomach resection, surgical mesh or sutures are usually used to reinforce the part subjected to the surgery, however, the material in common use at present has problems that easily result in foreign body inflammation and the inability to absorb tissue fluid. Furthermore, in a resection for the gastrointestinal tract, the part undergoing the resection may experience leakage of digestive fluid, body fluid, or blood, which can result in peritonitis, and during the repair process, peristalsis of the organ may occur. Therefore, an attach film has to be capable of perfectly fitting to the positions which are sutured.

However, because surgical mesh is made of hard material and has bad tissue adaptation, it cannot be tightly attached to soft tissue, and thus needs to be affixed using sutures, which can result in inconvenience during surgical operations. In addition, at present, the commercial surgical mesh appliances commonly used for auto-fixing, such as nails or hidden buttons, still often need be enhanced by use of sutures, and thus a secondary infection and leakage can easily occur at the location of the suture.

Therefore, at present, a novel patching film which can achieve a perfect fit and seal in the damp conditions within the body without the need for an additional fixative is needed.

### SUMMARY

The present disclosure provides a film composed of a polymer mixture, wherein the polymer mixture comprises: a hydrophobic composition including polycaprolactone (PCL); and at least one hydrophilic polymer selected from a group consisting of: alginate, gelatin, hyaluronic acid, polyvinyl alcohol (PVA), carboxymethyl cellulose (CMC), polyethylene glycol (PEG), collagen, demineralized bone matrix (DBM), bone morphogenetic protein (BMP), albumin, chitosan, fibrin, polyoxyethylene and polyvinylpyrrolidone. The weight ratio of the hydrophobic composition to the hydrophilic polymer is about 1:0.01-100. The film has the effect of preventing leakage from a surgical wound or a diffuse wound.

The present disclosure also provides a method for manufacturing a film, comprising: preparing a polymer mixture, wherein the method for preparing the polymer mixture comprises: preparing a hydrophobic solution, wherein a solute of the hydrophobic solution comprises polycaprolactone; and adding at least one hydrophilic polymer as a dispersing agent to the hydrophobic solution and mixing it with the hydrophobic solution, wherein the hydrophilic polymer is selected from a group consisting of: alginate, gelatin, hyaluronic acid, polyvinyl alcohol (PVA), carboxymethyl cellulose (CMC), polyethylene glycol (PEG), collagen, demineralized bone matrix (DBM), bone morphogenetic protein (BMP), albumin, chitosan, fibrin, polyoxyethylene and polyvinylpyrrolidone, wherein the weight ratio of the solute of the hydrophobic solution to the hydrophilic polymer is about 1:0.01-100. The method for manufacturing a film further comprises drying the polymer mixture to form a film.

The present disclosure further provides a double layer film, comprising: an attachment layer; and an anti-adhesion layer on a surface of the attachment layer and bonded thereto, wherein the attachment layer is the aforementioned film, and wherein the hydrophilic polymer, selected from a group consisting of: alginate, gelatin, collagen, demineralized bone matrix, bone morphogenetic protein, albumin, chitosan, fibrin, polyoxyethylene and polyvinylpyrrolidone, wherein the anti-adhesion layer is the aforementioned film, and wherein the hydrophilic polymer, selected from a group consisting of: hyaluronic acid, polyvinyl alcohol, carboxymethyl cellulose, and polyethylene glycol.

The present disclosure further provides a method for manufacturing a double layer film, comprising: (a) preparing a first polymer mixture and a second polymer mixture, wherein a method for preparing the first polymer mixture comprises: preparing a first hydrophobic solution, wherein a solute of the first hydrophobic solution comprises polycaprolactone; and adding at least one hydrophilic polymer as a first dispersing agent to the first hydrophobic solution and mixing it with the first hydrophobic solution to form the first polymer mixture, wherein the hydrophilic polymer, selected from a group consisting of: alginate, gelatin, collagen, demineralized bone matrix, bone morphogenetic protein, albumin, chitosan, fibrin, polyoxyethylene and polyvinylpyrrolidone, wherein the amount of first dispersing agent added is sufficient to let the first polymer mixture become a homogeneous mixture in colloidal form, wherein a method for preparing the second polymer mixture comprises: preparing a second hydrophobic solution, wherein a solute of the second hydrophobic solution comprises polycaprolactone; and adding at least one hydrophilic polymer as a second dispersing agent to the second hydrophobic solution and mixing it with the second hydrophobic solution to form the second polymer mixture, wherein the hydrophilic polymer is selected from a group consisting of: hyaluronic acid, polyvinyl alcohol, carboxymethyl cellulose, and polyethylene glycol, wherein the amount of second dispersing agent added is sufficient to let the second polymer mixture become a homogeneous mixture in colloidal form, (b) drying the first polymer mixture to form a film to form an attachment layer; and (c) drying the second polymer mixture on the attachment layer to form a film to form an anti-adhesion layer to complete the manufacture of the double layer film, and the solvent of the first hydrophobic solution and the solvent of the second hydrophobic solution are the same.

Moreover, the present disclosure provides a method for sealing a surgical wound or a diffuse wound, comprising: attaching the film or double layer film mentioned above to a surgical wound or a diffuse wound of a subject to seal the surgical wound or the diffuse wound.

A detailed description is given in the following embodiments with reference to the accompanying drawings.

### BRIEF DESCRIPTION OF DRAWINGS

The present invention can be more fully understood by reading the subsequent detailed description and examples with references made to the accompanying drawings, wherein:

FIG. 1 shows a schematic diagram of a film (double layer film) in one embodiment of the present disclosure;

FIG. 2A shows a situational schematic diagram of using a film in one embodiment of the present disclosure;

FIG. 2B shows a situational schematic diagram of using a film in another embodiment of the present disclosure;

FIGS. 3A to 3C respectively show viscosity analysis results for gelatin, hyaluronic acid, and polyvinyl alcohol;

FIGS. 4A to 4D show thermogravimetric analysis results of the films prepared in Examples 1-1 to 1-4 at different locations;

FIGS. 5A to 5C show thermogravimetric analysis results of the films prepared in Comparative Example 1, Example 2-1, and Example 3-1 at different locations;

FIGS. 6A to 6E show thermogravimetric analysis results for the double layer films prepared in Example 7-1 to Example 7-5, respectively;

FIG. 7 shows analysis result of Fourier transform infrared spectrometry (FT-IR) of the film prepared in Example 3-1;

FIGS. 8A to 8C show Fourier transform infrared spectrometry (FT-IR) analysis results for the double layer films prepared in Example 7-1, Example 7-2 and Example 7-3;

FIGS. 8D to 8F show Fourier transform infrared spectrometry (FT-IR) analysis results for the double layer films prepared by Example 7-1, Example 7-4 and Example 7-5;

FIG. 9 shows the results of a standard test of burst strength for the films prepared in Comparative Example 1, Example 2-1, and Example 3-1, and the commercial sealing film (TachoSil) and sealing patch (TissuePatch);

FIG. 10 shows the results of a standard test of burst strength for the films prepared in Examples 7-1 to 7-5 and the commercial sealing patch (TissuePatch);

FIG. 11 shows the results of a standard test of tensile properties for the films prepared in Comparative Example 1, Example 2-1, and Example 3-1, and the commercial sealing film (TachoSil) and sealing patch (TissuePatch);

FIG. 12 shows the results of a standard test of tensile properties for the films prepared in Examples 7-1 to 7-5 and the commercial sealing patch (TissuePatch).

FIG. 13A shows the results of a test of suture pullout of the double layer films prepared in Example 7-1, Example 7-2 and Example 7-3 and the commercial sealing patch (TissuePatch);

FIG. 13B shows the results of a test of suture pullout of the double layer films prepared in Example 7-1, Example 7-4 and Example 7-5 and the commercial sealing patch (TissuePatch);

FIG. 14A shows the results of a test of tear resistance of the double layer films prepared in Example 7-1, Example 7-2 and Example 7-3 and the commercial sealing patch (TissuePatch);

FIG. 14B shows the results of a test of tear resistance of the double layer films prepared in Example 7-1, Example 7-4 and Example 7-5 and the commercial sealing patch (TissuePatch);

FIGS. 15A and 15B show photographs of surface structures of the films prepared in Example 3-1 and prepared in Comparative Example 1;

FIGS. 16A to 16C show photographs of surface structures of the films prepared in Example 2-2, Example 3-2 and Example 7-1, respectively;

FIG. 17 shows the full roughness of the surfaces of the films prepared in Comparative Example 1, Example 2-1, and Example 3-1;

FIGS. 18A and 18B show the roughness of the anti-adhesion surfaces and attaching surfaces of the double layer films prepared in Example 7-1, Example 7-2 and Example 7-3 and the commercial sealing patch (TissuePatch);

FIGS. 19A and 19B shows the roughness of the anti-adhesion surfaces and attaching surfaces of the double layer films prepared in Example 7-1, Example 7-4 and Example 7-4 and the commercial sealing patch (TissuePatch);

FIG. 20 shows the results of thickness analysis of the films prepared in Examples 7-1 to 7-5 and the commercial sealing patch (TissuePatch);

FIG. 21 shows photographs of a surgical wound on the liver of a rat before implanting a film and 14 days after implanting the film, and shows the result of hematoxylin and eosin (H&E) stain for the tissue attached by the film after implanting the film for 14 days;

FIG. 22 shows photographs of a surgical wound on the stomach of a rat before implanting a film and 14 days after implanting the film, and shows the result of hematoxylin and eosin (H&E) stain for the tissue attached by the film after implanting the film for 14 days;

FIG. 23A shows adhesion scores for injured intestines of mice after no film treatment and with the attachment of anti-adhesion films prepared in Example 7-1, Example 7-5 and Example 7-6 and the sealing patch (TissuePatch) for 1 month;

FIG. 23B shows the photographs of intestine samples which are sampled from mice after their intestines are injured, which are treated without any film treatment or with the attachment of anti-adhesion film prepared in Example 7-1, Example 7-5, Example 7-6 and the sealing patch (TissuePatch) for 1 month and then sacrificed and surgery locations thereof are confirmed;

FIG. 23C shows the results of hematoxylin and eosin (H&E) stain and modified Gomori Trichrome (MGT) stain for intestine samples which are sampled from mice after their intestines are injured, which are treated without any film treatment or with the attachment of anti-adhesion film prepared in Example 7-1, Example 7-5, Example 7-6 and the sealing patch (TissuePatch) for 1 month and then sacrificed and surgery locations thereof are confirmed.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

In one aspect of the present disclosure, a film which is a biodegradable non-fiber form film, and can be well attached on a surgical wound or a diffuse wound without needing sutures or another fixing manner, and can prevent leakage of tissue fluid, is provided. Furthermore, in one aspect of the present disclosure, the present disclosure provides an adherent film which is fixative-free, and this film has the effect of preventing leakage.

In one embodiment, the film of the present disclosure mentioned above may be composed of a polymer mixture, but it is not limited thereto.

The foregoing polymer mixture may comprise, but is not limited to, a hydrophobic composition and at least one hydrophilic polymer.

The aforementioned hydrophobic composition may comprise polycaprolactone (PCL), but is not limited thereto. The molecular weight of the polycaprolactone may be about 5,000-150,000. In one embodiment, the molecular weight of the polycaprolactone is about 120,000.

In the present disclosure, examples of a suitable hydrophilic polymer may include, but are not limited to, alginate, gelatin, hyaluronic acid, polyvinyl alcohol (PVA), carboxymethyl cellulose (CMC), polyethylene glycol (PEG), collagen, demineralized bone matrix (DBM), bone morphogenetic protein (BMP), albumin, chitosan, fibrin, polyoxyethylene, polyvinylpyrrolidone, or a combination thereof.

In one embodiment, the hydrophilic polymer mentioned above may be in the form of a solid particle. In this embodiment, the particle size of the solid particle is about 1-1000 μm, but is not limited thereto.

In another embodiment, the hydrophilic polymer mentioned above may be dissolved in a solvent to be in the form of a liquid polymer. In this embodiment, examples of said solvent may include, but are not limited to, water, ethanol, acetone, an acidic solution, an alkaline solution, and a buffer solution. Moreover, in this embodiment, the limiting viscosity of the liquid polymer may be about 1-200 dl/g, but it is not limited thereto.

In addition, in the film of the present disclosure, the weight ratio of the hydrophobic composition to the hydrophilic polymer may be about 1:0.01-100, but it is not limited thereto. In one embodiment, the weight ratio of the hydrophobic composition to the hydrophilic polymer may be about 1:0.625. In another embodiment, the weight ratio of the hydrophobic composition to the hydrophilic polymer may be about 1:1.25.

In one embodiment, in the film of the present disclosure, the hydrophilic polymer mentioned above is alginate. Moreover, in this embodiment, the weight ratio of the preceding hydrophobic composition to the alginate is about 1:0.05-80, such as 1:0.0625-60, but is not limited thereto. In one specific embodiment, the weight ratio of the preceding hydrophobic composition to the alginate is about 1:0.0625, 1:0.625, 1:20, 1:60, etc.

In another embodiment, in the film of the present disclosure, the hydrophilic polymer mentioned above is gelatin. The molecular weight of the gelatin is about 10,000-200,000, but it is not limited thereto. In this embodiment, the weight ratio of the foregoing hydrophobic composition to the gelatin is about 1:0.05-80, such as 1:0.0625-60, but it is not limited thereto. In one specific embodiment, the weight ratio of the foregoing hydrophobic composition to the gelatin is about 1:0.6167, 1:0.74, 1:0.925, 1:1.25, etc.

In another embodiment, in the film of the present disclosure, the hydrophilic polymer mentioned above is hyaluronic acid. The molecular weight of the hyaluronic acid is about 500,000-5,000,000, but it is not limited thereto. In this embodiment, the weight ratio of the foregoing hydrophobic composition to the hyaluronic acid is about 1:0.005-80, such as 1:0.02-60, but it is not limited thereto. In one specific embodiment, the weight ratio of the foregoing hydrophobic composition to the hyaluronic acid is about 1:0.0167, 1:0.02, 1:0.025, etc.

In yet another embodiment, in the film of the present disclosure, the hydrophilic polymer mentioned above is a combination of hyaluronic acid and polyvinyl alcohol. The molecular weight of the hyaluronic acid is about 500,000-5,000,000, but it is not limited thereto, and the molecular weight of the polyvinyl alcohol is about 2,000-400,000, but it is not limited thereto. In this embodiment, the weight ratio of the foregoing hydrophobic composition to the combination of hyaluronic acid and polyvinyl alcohol is about 1:0.01-80, such as 1:0.02-60, but it is not limited thereto. In one specific embodiment, the weight ratio of the foregoing hydrophobic composition to the combination of hyaluronic acid and polyvinyl alcohol is about 1:0.05, 1:0.075, etc.

Moreover, in one embodiment, in the film of the present disclosure, the hydrophilic polymer mentioned above is a combination of carboxymethyl cellulose and polyethylene glycol. The molecular weight of the carboxymethyl cellulose is about 10,000-300,000, but it is not limited thereto, and the molecular weight of the polyethylene glycol is about 2,000-400,000, but it is not limited thereto. In this embodiment, the weight ratio of the foregoing hydrophobic composition to the combination of carboxymethyl cellulose and polyethylene glycol is about 1:0.1-80, such as 1:0.5-60, but it is not limited thereto. In one specific embodiment, the weight ratio of the foregoing hydrophobic composition to the combination of carboxymethyl cellulose and polyethylene glycol is about 1:0.8, etc.

In one embodiment of the present disclosure, in the preceding polymer mixture which composes the film of the present disclosure, the hydrophobic composition is composed of polycaprolactone. In this embodiment, the weight ratio of the polycaprolactone to the hydrophilic polymer mentioned above may be about 1:0.01-80, such as 1:0.01-60, such as 1:0.0167, 1:0.02, 1:0.025, 1:0.05, 1:0.075, 1:0.6167, 1:0.74, 1:0.8, 1:0.925, 1:1.25, but it is not limited thereto.

In another embodiment of the present disclosure, in the preceding polymer mixture which composes the film of the present disclosure, the hydrophobic composition, in addition to polycaprolacton, may further comprise at least one hydrophobic polymer. The hydrophobic polymer mentioned herein may comprise polylactic acid (PLA), poly(lactic-co-glycolic acid) (PLGA), poly(glycolic acid) (PGA), polyhydroxybutyrate, (PHB), polydioxanone (PDS), poly(propylene fumarate) (PPF), polyanhydrides, polyacetals, poly(ortho esters), polycarbonates, polyurethanes, polyphosphazenes, polyphosphoester), or a combination thereof, but it is not limited thereto. In the hydrophobic composition mentioned above, the weight ratio of the polycaprolactone to the hydrophilic polymer mentioned above may be about 1:0.01-10, such as 1:0.25, but it is not limited thereto. Furthermore, in this embodiment, the weight ratio of the hydrophobic composition mentioned above to the hydrophilic polymer mentioned above may be about 1:0.05-80, such as 1:0.0625-60, but it is not limited thereto. In one specific embodiment, the weight ratio of the hydrophobic composition mentioned above to the hydrophilic polymer mentioned above may be about 1:0.0625, 1:0.625, 1:0.925, 1:1.25, 1:20 or 1:60. Moreover, in this embodiment, the weight ratio of the polycaprolactone to the hydrophilic polymer mentioned above may be about 1:0.02-90, such as 1:0.07-75, but it is not limited thereto. In one specific embodiment, the weight ratio of the polycaprolactone to the hydrophilic polymer mentioned above may be about 1:0.078125, 1:0.78125, 1:1.1.5625, 1:31.25 or 1:75, etc.

In the embodiment in which the hydrophobic composition, in addition to polycaprolacton, may further comprise at least one hydrophobic polymer, the molecular weight of the polycaprolactone may be about 5,000-150,000, but it is not limited thereto. Furthermore, in this embodiment, the weight ratio of the hydrophobic composition to the hydrophilic polymer may be about 1:0.05-80, but it is not limited thereto.

In addition, in the embodiment in which the hydrophobic composition, in addition to polycaprolacton, may further comprise at least one hydrophobic polymer, the hydrophilic polymer mentioned above may be in the form of a solid particle, and the particle size of the solid particle may be about 1-1000 μm, but it is not limited thereto. Alternatively, in this embodiment, the hydrophilic polymer may be dissolved in a solvent to be in the form of liquid polymer, and examples of the solvent may include, but are not limited to, water, ethanol, acetone, an acidic solution, an alkaline solution, and a buffer solution. In addition, in this embodiment, the limiting viscosity of said liquid polymer may be about 1-200 dl/g, but it is not limited thereto.

Furthermore, in the embodiment in which the hydrophobic composition, in addition to polycaprolacton, may further comprise at least one hydrophobic polymer, in the preceding polymer mixture, the hydrophilic polymer mentioned above is alginate, and the weight ratio of the hydrophobic composition to the alginate is about 1:0.05-80, but is not limited thereto. Alternatively, in this embodiment, the hydrophilic polymer is gelatin, and the weight ratio of the hydrophobic composition to the gelatin is about 1:0.05-80, but it is not limited thereto.

Moreover, in one embodiment, in the preceding polymer mixture which composes the film of the present disclosure, the hydrophobic composition, in addition to polycaprolacton, may further comprise at least one hydrophobic polymer, and the hydrophobic polymer may be poly(lactic-co-glycolic acid). In this embodiment, the weight ratio of the hydrophobic composition to the hydrophilic polymer may be about 1:0.05-80, such as 1:0.0625-60, but is not limited thereto. In one specific embodiment, the weight ratio of the hydrophobic composition to the hydrophilic polymer mentioned above may be about 1:0.0625, 1:0.625, 1:0.925, 1:1.25, 1:20 or 1:60. Furthermore, in this embodiment, the weight ratio of the polycaprolactone to the hydrophilic polymer mentioned above may be about 1:0.02-90, such as 1:0.07-75, but it is not limited thereto. In one specific embodiment, the weight ratio of the polycaprolactone to the hydrophilic polymer mentioned above may be about 1:0.078125, 1:0.78125, 1:1.1.5625, 1:31.25 or 1:75, etc.

In the embodiment in which the hydrophobic composition, in addition to polycaprolacton, may further comprise poly(lactic-co-glycolic acid), the molecular weight of the polycaprolactone may be about 5,000-150,000, such as 120,000, but it is not limited thereto.

Furthermore, in the embodiment in which the hydrophobic composition, in addition to polycaprolacton, may further comprise poly(lactic-co-glycolic acid), the hydrophilic polymer may be in the form of a solid particle, and the particle size of the solid particle is about 1-1000 μm, but is not limited thereto. Alternatively, in this embodiment, the hydrophilic polymer may be dissolved in a solvent to be in the form of liquid polymer, and examples of said solvent may include, but are not limited to, water, ethanol, acetone, an acidic solution, an alkaline solution, and a buffer solution, and the limiting viscosity of the liquid polymer may be about 1-200 dl/g, but it is not limited thereto.

In the preceding embodiment in which the hydrophobic composition, in addition to polycaprolacton, may further comprise poly(lactic-co-glycolic acid), in one specific embodiment, in the film of the present disclosure, the hydrophilic polymer mentioned above is alginate, and the weight ratio of the hydrophobic composition mentioned above to the alginate is about 1:0.05-80, such as, about 1:0.0625, 1:0.625, 1:1.25, 1:20 or 1:60, but is not limited thereto. In addition, in another specific embodiment, in the film of the present disclosure, the hydrophilic polymer mentioned above is gelatin. In this embodiment, the weight ratio of the hydrophobic composition mentioned above to the gelatin is about 1:0.05-80, such as 1:0.925, 1:1.25, but it is not limited thereto.

In another aspect of the present disclosure, a method for manufacturing a film is provided, wherein said film is a non-fiber form film, and can be attached on a surgical wound or a diffuse wound without the need for sutures or any other fixing manner, and can prevent leakage of tissue fluid.

In one embodiment, the method for manufacturing a film mentioned above may comprise the following steps, but it is not limited thereto.

First, a polymer mixture is prepared.

Next, the polymer mixture is dried to form a film.

Moreover, a method for preparing the preceding polymer mixture may comprise the following steps, but is not limited thereto.

First, a hydrophobic solution is prepared, and a solute of the hydrophobic solution may comprise, but is not limited to, polycaprolactone. The molecular weight of the polycaprolactone may be about 5,000-150,000, but is not limited thereto. In one embodiment, the molecular weight of the polycaprolactone may be 120,000. Furthermore, in one embodiment, the hydrophobic solution is formed by dissolving the polycaprolactone in a solvent. Examples of the solvent mentioned above may include acetone, acetic acid, chloroform, methanol, dichloromethane, dimethylformamide, dioxane, ethyl acetate, formic acid, hexafluoroisopropanol, 1-methyl-2-pyrrolidone, tetrahydrofuran, toluene, and mixture solutions thereof, but it is not limited thereto.

Next, at least one hydrophilic polymer as a dispersing agent is added to the hydrophobic solution and mixed with the hydrophobic solution. The weight ratio of the solute of the foregoing hydrophobic solution to the hydrophilic polymer mentioned above may be about 1:0.01-100, but it is not limited thereto. In one embodiment, the weight ratio of the solute of the hydrophobic solution to the hydrophilic polymer may be about 1:0.625. In another embodiment, the weight ratio of the solute of the hydrophobic solution to the hydrophilic polymer may be about 1:1.25.

Examples of suitable hydrophilic polymer may include, but are not limited to, alginate, gelatin, hyaluronic acid, polyvinyl alcohol (PVA), carboxymethyl cellulose (CMC), polyethylene glycol (PEG), collagen, demineralized bone matrix (DBM), bone morphogenetic protein (BMP), albumin, chitosan, fibrin, polyoxyethylene, polyvinylpyrrolidone, or a combination thereof.

In one embodiment, the hydrophilic polymer may be in the form of a solid particle. In this embodiment, the particle size of the solid particle is about 1-1000 μm, but it is not limited thereto.

In another embodiment, the hydrophilic polymer may be dissolved in a solvent to be in the form of liquid polymer. In this embodiment, examples of said solvent may include, but are not limited to, water, ethanol, acetone, an acidic solution, an alkaline solution, and a buffer solution. Furthermore, in this embodiment, the limiting viscosity of the liquid polymer may be about 1-200 dl/g, but it is not limited thereto.

The viscosity of the polymer mixture may be about 300-700 CP, such as about 300 CP, 348 CP, 350 CP, 400 CP, 402 CP, 450 CP, 500 CP, 550 CP, 582 CP, 600 CP, 621 CP, 650 CP, 700 CP, but it is not limited thereto. Furthermore, the dispersing agent and the hydrophobic solution can be mixed at a stirring rate of about 30-100 rpm to form the polymer mixture. In one embodiment, the dispersing agent and the hydrophobic solution can be mixed at a stirring rate of about 45-80 rpm to form the polymer mixture. In addition, the time required for mixing the dispersing agent and the hydrophobic solution may be about 30-300 seconds, such as about 30-120 seconds, 45-90 seconds, 30-60 seconds, but it is not limited thereto. Stirring rate and time are related to the viscosity of the polymer mixture and whether all ingredients in the polymer mixture can be uniformly mixed or not, and if the stirring rate is lower than 30 rpm or the stirring time is less than 30 seconds, it may cause the ingredients in the polymer mixture to be unable to mix uniformly.

In one embodiment, the hydrophilic polymer mentioned above is alginate. Moreover, in this embodiment, the weight ratio of the solute of the foregoing hydrophobic solution to the alginate may be about 1:0.05-80, such as 1:0.0625-60, but is not limited thereto. In one specific embodiment, the weight ratio of the solute of the foregoing hydrophobic solution to the alginate may be about 1:0.0625, 1:0.625, 1:20, 1:60, etc.

In another embodiment, the hydrophilic polymer mentioned above is gelatin. The molecular weight of the gelatin is about 10,000-200,000, but it is not limited thereto. In this embodiment, the weight ratio of the solute of the preceding hydrophobic solution to the gelatin may be about 1:0.05-80, such as 1:0.0625-60, but is not limited thereto. In one specific embodiment, the weight ratio of the solute of the preceding hydrophobic solution to the gelatin may be about 1:0.6167, 1:0.74, 1:0.925, 1:1.25, etc.

In another embodiment, the hydrophilic polymer mentioned above is hyaluronic acid. The molecular weight of the hyaluronic acid is about 500,000-5,000,000, but it is not limited thereto. In this embodiment, the weight ratio of the foregoing hydrophobic composition to the hyaluronic acid is about 1:0.005-80, such as 1:0.02-60, but it is not limited thereto. In one specific embodiment, the weight ratio of the foregoing hydrophobic composition to the hyaluronic acid is about 1:0.0167, 1:0.02, 1:0.025, etc.

In yet another embodiment, the hydrophilic polymer mentioned above is a combination of hyaluronic acid and polyvinyl alcohol. The molecular weight of the hyaluronic acid is about 500,000-5,000,000, but it is not limited thereto, and the molecular weight of the polyvinyl alcohol is about 2,000-400,000, but it is not limited thereto. In this embodiment, the weight ratio of the foregoing hydrophobic composition to the combination of hyaluronic acid and polyvinyl alcohol is about 1:0.01-80, such as 1:0.02-60, but it is not limited thereto. In one specific embodiment, the weight ratio of the foregoing hydrophobic composition to the combination of hyaluronic acid and polyvinyl alcohol is about 1:0.05, 1:0.075, etc.

Moreover, in one embodiment, the hydrophilic polymer mentioned above is a combination of carboxymethyl cellulose and polyethylene glycol. The molecular weight of the carboxymethyl cellulose is about 10,000-300,000, but it is not limited thereto, and the molecular weight of the polyethylene glycol is about 2,000-400,000, but it is not limited thereto. In this embodiment, the weight ratio of the foregoing hydrophobic composition to the combination of carboxymethyl cellulose and polyethylene glycol is about 1:0.1-80, such as 1:0.5-60, but it is not limited thereto. In one specific embodiment, the weight ratio of the foregoing hydrophobic composition to the combination of carboxymethyl cellulose and polyethylene glycol is about 1:0.8, etc.

In one embodiment of the present disclosure, in the preceding polymer mixture, the solute of the hydrophobic solution is composed of polycaprolactone. In this embodiment, the weight ratio of the polycaprolactone to the hydrophilic polymer mentioned above may be about 1:0.01-80, such as 1:0.01-60, such as 1:0.0167, 1:0.02, 1:0.025, 1:0.05, 1:0.075, 1:0.6167, 1:0.74, 1:0.8, 1:0.925, 1:1.25, but it is not limited thereto.

In one embodiment, in the polymer mixture, the solute of the hydrophobic solution, in addition to polycaprolacton, may further comprise at least one hydrophobic polymer. The hydrophobic polymer mentioned herein may comprise polylactic acid (PLA), poly(lactic-co-glycolic acid) (PLGA), poly(glycolic acid) (PGA), polyhydroxybutyrate (PHB), polydioxanone (PDS), poly(propylene fumarate) (PPF), polyanhydrides, polyacetals, poly(ortho esters), polycarbonates, polyurethanes, polyphosphazenes, polyphosphoester, or a combination thereof, but it is not limited thereto. In the solute of the hydrophobic solution, the weight ratio of the polycaprolactone to the at least one hydrophobic polymer mentioned above may be about 1:0.01-10, such as 1:0.25, but it is not limited thereto. Furthermore, in this embodiment, the weight ratio of the solute of the preceding hydrophobic solution to the hydrophilic polymer mentioned above may be about 1:0.05-80, such as 1:0.0625-60, but it is not limited thereto. In one specific embodiment, the weight ratio of the solute of the preceding hydrophobic solution to the hydrophilic polymer mentioned above may be about 1:0.0625, 1:0.625, 1:0.925, 1:1.25, 1:20 or 1:60. In addition, in this embodiment, the weight ratio of the polycaprolactone to the hydrophilic polymer mentioned above may be about 1:0.02-90, such as 1:0.07-75, but it is not limited thereto. In one specific embodiment, the weight ratio of the polycaprolactone to the hydrophilic polymer mentioned above may be about 1:0.078125, 1:0.78125, 1:1.1.5625, 1:31.25 or 1:75, etc.

In the embodiment in which the solute of the hydrophobic solution, in addition to polycaprolacton, may further comprise at least one hydrophobic polymer, the hydrophobic solution may be formed by a method, and the method may comprise dissolving the polycaprolactone in a first solvent to form a first solution, and dissolving the hydrophobic polymer in a second solvent to form a second solution, and then mixing the first solution with the second solution to form the hydrophobic solution. Furthermore, the first solvent and the second solvent may be the same or different.

Examples of the foregoing first solvent may include acetone, acetic acid, chloroform, methanol, dichloromethane, dimethylformamide, dioxane, ethyl acetate, formic acid, hexafluoroisopropanol, 1-methyl-2-pyrrolidone, tetrahydrofuran, toluene, and combinations thereof, but they are not limited thereto. The second solvent mentioned above may comprise, but is not limited to, acetone, acetic acid, chloroform, methanol, dichloromethane, dimethylformamide, dioxane, ethyl acetate, formic acid, hexafluoroisopropanol, 1-methyl-2-pyrrolidone, tetrahydrofuran, toluene or a combination thereof.

Alternatively, in the embodiment in which the solute of the hydrophobic solution, in addition to polycaprolacton, may further comprise at least one hydrophobic polymer, the hydrophobic solution may be formed by another method, and the method may comprise dissolving the polycaprolactone and the hydrophobic polymer mentioned above in the same solvent to form said hydrophobic solution. The solvent mentioned herein may comprise acetone, acetic acid, chloroform, methanol, dichloromethane, dimethylformamide, dioxane, ethyl acetate, formic acid, hexafluoroisopropanol, 1-methyl-2-pyrrolidone, tetrahydrofuran, toluene or a combination thereof, but it is not limited thereto.

Moreover, in the embodiment in which the solute of the hydrophobic solution, in addition to polycaprolacton, may further comprise at least one hydrophobic polymer, the molecular weight of the polycaprolactone may be about 5,000-150,000, but is not limited thereto. In addition, in this embodiment, the weight ratio of the solute of the hydrophobic solution to the hydrophilic polymer may be about 1:0.05-80, but is not limited thereto.

Furthermore, in the embodiment in which the solute of the hydrophobic solution, in addition to polycaprolactone, may further comprise at least one hydrophobic polymer, the hydrophilic polymer mentioned above may be in the form of a solid particle, and the particle size of the solid particle may be about 1-1000 μm, but is not limited thereto. Alternatively, in this embodiment, the hydrophilic polymer mentioned above may be dissolved in a solvent to be in the form of liquid polymer, and examples of said solvent may include, but are not limited to, water, ethanol, acetone, an acidic solution, an alkaline solution, and a buffer solution. In addition, in this embodiment, the limiting viscosity of the liquid polymer may be about 1-200 dl/g, but it is not limited thereto.

Moreover, in the preceding embodiment in which the solute of the hydrophobic solution, in addition to polycaprolacton, may further comprise at least one hydrophobic polymer, in the polymer mixture mentioned above, the hydrophilic polymer mentioned above is alginate, and the weight ratio of the solute of the hydrophobic solution to the alginate may be about 1:0.05-80, but is not limited thereto. Alternatively, in this embodiment, the hydrophilic polymer is gelatin, and the weight ratio of the solute of the hydrophobic solution to the gelatin may be about 1:0.05-80, but is not limited thereto.

In addition, in one embodiment, in the polymer mixture, the solute of the hydrophobic solution, in addition to polycaprolacton, may further comprise at least one hydrophobic polymer, and the at least one hydrophobic polymer may be poly(lactic-co-glycolic acid). In this embodiment, the weight ratio of the solute of the hydrophobic solution to the hydrophilic polymer may be about 1:0.05-80, such as 1:0.0625-60, but is not limited thereto. In one specific embodiment, the weight ratio of the solute of the hydrophobic solution to the hydrophilic polymer mentioned above may be about 1:0.0625, 1:0.625, 1:0.925, 1:1.25, 1:20 or 1:60. Moreover, in this embodiment, the weight ratio of the polycaprolactone to the hydrophilic polymer mentioned above may be about 1:0.02-90, such as, 1:0.07-75, but is not limited thereto. In one specific embodiment, the weight ratio of the polycaprolactone to the hydrophilic polymer mentioned above may be about 1:0.078125, 1:0.78125, 1:1.1.5625, 1:31.25 or 1:75, etc.

In the preceding embodiment in which the solute of the hydrophobic solution, in addition to polycaprolacton, may further comprise poly(lactic-co-glycolic acid), the molecular weight of the polycaprolactone may be about 5,000-150,000, such as 120,000, but it is not limited thereto.

Moreover, in the preceding embodiment in which the solute of the hydrophobic solution, in addition to polycaprolacton, may further comprise poly(lactic-co-glycolic acid), the hydrophilic polymer may be in the form of a solid particle, and the particle size of the solid particle is about 1-1000 μm, but it is not limited thereto. Alternatively, in this embodiment, the hydrophilic polymer may be dissolved in a solvent to be in the form of a liquid polymer, and examples of said solvent may include, but are not limited to, water, ethanol, acetone, an acidic solution, an alkaline solution, and a buffer solution, and the limiting viscosity of the liquid polymer may be about 1-200 dl/g, but it is not limited thereto.

In the preceding embodiment in which the solute of the hydrophobic solution, in addition to polycaprolacton, may further comprise poly(lactic-co-glycolic acid), in one specific embodiment, in the film of the present disclosure, the hydrophilic polymer mentioned above is alginate, and the weight ratio of the solute of the hydrophobic solution mentioned above to the alginate may be about 1:0.05-80, such as about 1:0.0625, 1:0.625, 1:1.25, 1:20 or 1:60, but is not limited thereto. Moreover, in another specific embodiment, in the film of the present disclosure, the hydrophilic polymer mentioned above is gelatin. In this embodiment, the weight ratio of the solute of the preceding hydrophobic solution to the gelatin may be about 1:0.05-80, such as 1:0.925, 1:1.25, but is not limited thereto.

It should be noted that in the method for manufacturing a film of the present disclosure, by using the hydrophilic polymer as a dispersing agent, the ingredients of the polymer mixture can be uniformly distributed, and a film with an even surface can be formed.

In addition, in the method for manufacturing a film of the present disclosure, a manner for drying the polymer mixture has no particular limitation, only that the polymer mixture is able to form a film. In one embodiment, the polymer mixture may be poured onto a plate, and then scraped with a scraper to perform a film scraping procedure, and after that, dried to form a film.

Furthermore, in one embodiment, the method for manufacturing a film of the present disclosure may further comprise performing a film stripping process after the polymer mixture is dried to form a film.

The film stripping process mentioned above has no particular limitation, only that the film can be detached from the material to which it is attached. In one embodiment, the film stripping process comprises immersing the film with the material to which it is attached in a film-stripping solution to make the film detach from the material to which it is attached.

Examples of the film-stripping solution may include, but are not limited to, ethanol, glycerol, soap base, and polyethylene glycol.

Moreover, the weight ratio of the film to the film-stripping solution may be about 1-20:10-2000, but it is not limited thereto.

In another aspect of the present disclosure, a film which is manufactured by any one of the preceding methods for manufacturing a film of the present disclosure is provided.

In one embodiment, any film of the present disclosure mentioned above may have a thickness of about 1-3000 μm. Moreover, in one embodiment, the full roughness (Rz) of any film of the present disclosure mentioned above may be about 1-100 μm.

Furthermore, the burst pressure of any film of the present disclosure mentioned above may be about 5-1000 cm-H₂O.

In addition, the tensile strength of any film of the present disclosure mentioned above may be about 5-3000 kPa.

In another aspect of the present disclosure, a double layer film which is a biodegradable non-fiber form anti-adhesion layer, and can be well attached on a surgical wound or a diffuse wound without needing sutures or another fixing manner, and can prevent leakage of tissue fluid, is provided. Furthermore, in one aspect of the present disclosure, the present disclosure provides an adherent film which is fixative-free, and this film has the effects of anti-adhesion and preventing leakage.

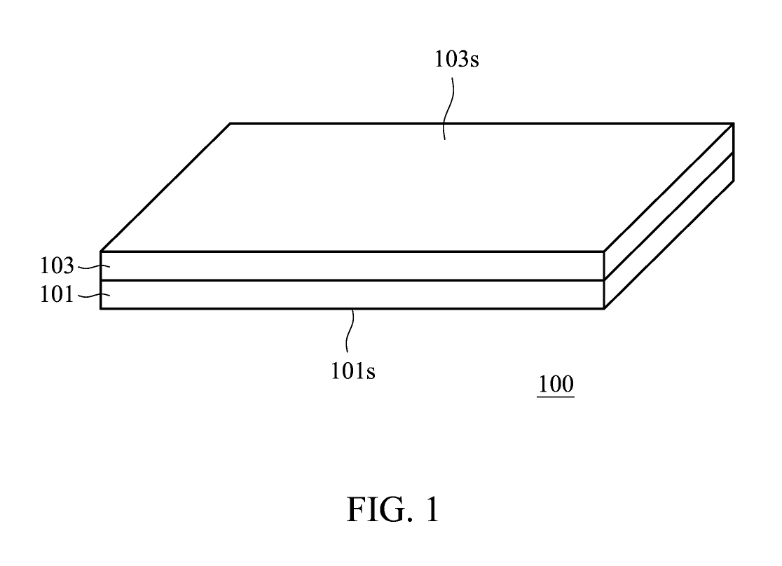

FIG. 1 shows a schematic diagram of a double layer film one embodiment of the present disclosure.

Please Refer to FIG. 1. A double layer film 100 of the present disclosure may comprise an attachment layer 101 and an anti-adhesion layer 103 on a surface of the attachment layer 101 and bonded thereto. Another surface of the attachment layer 101s is used to attach to a surface to be fixed or repaired while a surface of the anti-adhesion layer 103s has an anti-adhesion effect and can used be to prevent occurrence of tissue adhesion. A thickness ratio of the attachment layer 101 and the anti-adhesion layer 103 may be about 1:0.001-5, but it is not limited thereto. Moreover, the weight ratio of the attachment layer 101 and the anti-adhesion layer 103 may be about 1:0.001-5, but it is not limited thereto.

The attachment layer 101 in the double layer film of the present disclosure may be any of the aforementioned films of the present disclosure, although the hydrophilic polymer therein needs to be different from the hydrophilic polymer contained by the anti-adhesion layer 103.

The anti-adhesion layer 103 in the double layer film of the present disclosure may be any of the aforementioned films of the present disclosure, although the hydrophilic polymer therein needs to be a hydrophilic polymer with anti-adhesion effects, such as hyaluronic acid, polyvinyl alcohol, carboxymethyl cellulose, polyethylene glycol, and combinations thereof, but it is not limited thereto.

In one embodiment, examples of the hydrophilic polymer contained by the attachment layer 101 in the double layer film 100 of the present disclosure may comprise, but is not limited to, alginate, gelatin, collagen, demineralized bone matrix, bone morphogenetic protein, albumin, chitosan, fibrin, polyoxyethylene, polyvinylpyrrolidone and a combination thereof while examples of the hydrophilic polymer contained by the attachment layer 101 in the double layer film 100 of the present disclosure may comprise, but is not limited to, hyaluronic acid, polyvinyl alcohol, carboxymethyl cellulose, polyethylene glycol, or a combination thereof.

In the double layer film 100 of the present disclosure, the weight ratio of the total amount of the hydrophobic composition to the total amount of the hydrophilic polymer is about 1:0.1-2, such as 1:0.3167, 1:0.38, 1:0.475, 1:0.4875, 1:0.5, 1:0.8625, but it is not limited thereto.

In one embodiment, in the attachment layer 101, the content of the hydrophilic polymer thereof is about 10-80 wt %, such as 21 wt %, 35 wt %, but it is not limited thereto.

In one embodiment, in the anti-adhesion layer 103, the content of the hydrophilic polymer thereof is about 0.1-30 wt %, such as 0.5 wt %, 0.8 wt %, but it is not limited thereto.

In one embodiment, in the attachment layer 101, the hydrophilic polymer thereof may be gelatin. The molecular weight of the gelatin is about 10,000-200,000, but it is not limited thereto.

In one embodiment, in the anti-adhesion layer 103, the hydrophilic polymer thereof may be hyaluronic acid, and the molecular weight of the hyaluronic acid is about 500,000-5,000,000, but it is not limited thereto. In another embodiment, in the anti-adhesion layer 103, the hydrophilic polymer thereof may be a combination of hyaluronic acid and polyvinyl alcohol, and the molecular weight of the hyaluronic acid is about 500,000-5,000,000, but it is not limited thereto, and the molecular weight of the polyvinyl alcohol is about 2,000-400,000, but it is not limited thereto. In this embodiment, the weight ratio of the hyaluronic acid to the polyvinyl alcohol is about 1:0.5-5, such as 1:1, 1:2, but it is not limited thereto. In yet another embodiment, in the anti-adhesion layer 103, the hydrophilic polymer thereof may be a combination of carboxymethyl cellulose and polyethylene glycol, and the molecular weight of the carboxymethyl cellulose is about 10,000-300,000, but it is not limited thereto, and the molecular weight of the polyethylene glycol is about 2,000-400,000, but it is not limited thereto. In this embodiment, the weight ratio of the carboxymethyl cellulose to polyethylene glycol is about 1:0.1-30, such as 1:15, but it is not limited thereto.

Furthermore, in another aspect of the present disclosure, a method for manufacturing a double layer film which may be used to manufacture the foregoing double layer film of the present disclosure is provided.

The method for manufacturing a double layer film mentioned above may comprise, but is not limited to the following steps.

First, a first polymer mixture and a second polymer mixture are prepared.

A method for preparing the first polymer mixture may comprise the following steps, but it is not limited thereto.

First, a first hydrophobic solution is prepared, and a solute of the first hydrophobic solution may comprise, but it is not limited to, polycaprolactone. The molecular weight of the polycaprolactone may be about 5,000-150,000, but it is not limited thereto. In one embodiment, the molecular weight of the polycaprolactone may be about 120,000. Furthermore, in one embodiment, the first hydrophobic solution is formed by dissolving polycaprolactone in a solvent. Examples of the solvent may comprise acetone, acetic acid, chloroform, methanol, dichloromethane, dimethylformamide, dioxane, ethyl acetate, formic acid, hexafluoroisopropanol, 1-methyl-2-pyrrolidone, tetrahydrofuran, toluene, a mixture solution thereof, but they are not limited thereto.

Next, at least one hydrophilic polymer as a first dispersing agent is added to the first hydrophobic solution and mixed with the first hydrophobic solution to form the first polymer mixture, and the amount of first dispersing agent added is sufficient to let the first polymer mixture become a homogeneous mixture in colloidal form. The hydrophilic polymer mentioned herein may be in the form of a solid particle or may be dissolved in a solvent to be in the form of liquid polymer.

The viscosity of the first polymer mixture may be about 300-700 CP, such as about 300 CP, 348 CP, 350 CP, 400 CP, 402 CP, 450 CP, 500 CP, 550 CP, 582 CP, 600 CP, 621 CP, 650 CP, 700 CP, but it is not limited thereto. Furthermore, the first dispersing agent and the first hydrophobic solution can be mixed at a stirring rate of about 30-100 rpm to form the first polymer mixture. In one embodiment, the first dispersing agent and the first hydrophobic solution can be mixed at a stirring rate of about 45-80 rpm to form the first polymer mixture. In addition, the time required for mixing the first dispersing agent and the first hydrophobic solution may be about 30-300 seconds, such as about 30-120 seconds, 45-90 seconds, 30-60 seconds, but it is not limited thereto. Stirring rate and time are related to the viscosity of the polymer mixture and whether all ingredients in the polymer mixture can be uniformly mixed or not, and if the stirring rate is lower than 30 rpm or the stirring rate is less than 30 seconds, it may cause the ingredients in the polymer mixture to be unable to mix uniformly.

In the first polymer mixture, the solid content is about 10-60 wt %, such as 20 wt %, 35 wt %, but it is not limited thereto. Moreover, the weight ratio of the solute of the first hydrophobic solution to the first dispersing agent is about 1:0.1-5. In one embodiment, the weight ratio of the solute of the first hydrophobic solution to the first dispersing agent is about 1:0.925. In another embodiment, the weight ratio of the solute of the first hydrophobic solution to the first dispersing agent is about 1:0.74. In yet another embodiment, the weight ratio of the solute of the first hydrophobic solution to the first dispersing agent is about 1:0.6167.

Furthermore, a method for preparing the second polymer mixture may comprise the following steps, but it is not limited thereto.

First, a second hydrophobic solution is prepared, and a solute of the second hydrophobic solution may comprise, but it is not limited to, polycaprolactone. The molecular weight of the polycaprolactone may be about 5,000-150,000, but it is not limited thereto. In one embodiment, the molecular weight of the polycaprolactone may be about 120,000. Furthermore, in one embodiment, the second hydrophobic solution is formed by dissolving polycaprolactone in a solvent. Examples of the aforementioned solvent may comprise acetone, acetic acid, chloroform, methanol, dichloromethane, dimethylformamide, dioxane, ethyl acetate, formic acid, hexafluoroisopropanol, 1-methyl-2-pyrrolidone, tetrahydrofuran, toluene, a mixture solution thereof, but they are not limited thereto.

Next, at least one hydrophilic polymer as a second dispersing agent is added to the second hydrophobic solution and mixed with the second hydrophobic solution to form the second polymer mixture, and the amount of second dispersing agent added is sufficient to let the second polymer mixture become a homogeneous mixture in colloidal form. The hydrophilic polymer mentioned herein may be in the form of a solid particle or may be dissolved in a solvent to be in the form of liquid polymer.

The viscosity of the second polymer mixture may be about 300-700 CP, such as about 300 CP, 348 CP, 350 CP, 400 CP, 402 CP, 450 CP, 500 CP, 550 CP, 582 CP, 600 CP, 621 CP, 650 CP, 700 CP, but it is not limited thereto. Furthermore, the second dispersing agent and the second hydrophobic solution can be mixed at a stirring rate of about 30-100 rpm to form the second polymer mixture. In one embodiment, the second dispersing agent and the second hydrophobic solution can be mixed at a stirring rate of about 45-80 rpm to form the second polymer mixture. In addition, the time required for mixing the second dispersing agent and the second hydrophobic solution may be about 30-300 seconds, such as about 30-120 seconds, 45-90 seconds, 30-60 seconds, but it is not limited thereto. Stirring rate and time are related to the viscosity of the polymer mixture and whether all ingredients in the polymer mixture can be uniformly mixed or not, and if the stirring rate is lower than 30 rpm or stirring rate is less than 30 seconds, it may cause the ingredients in the polymer mixture to be unable to be mixed uniformly.

In the second polymer mixture, the solid content is about 0.1-30 wt %, such as 2 wt %, 8 wt %, but it is not limited thereto. Moreover, the weight ratio of the solute of the second hydrophobic solution to the second dispersing agent is about 1:0.01-10, such as 1:0.0167, 1:0.02, 1:0.025, 1:0.05, 1:0.075, 1:0.8, but it is not limited thereto.

The foregoing first dispersing agent and second dispersing agent are different. And the hydrophilic polymer used by the second dispersing agent needs to have anti-adhesion effects, such as hyaluronic acid, polyvinyl alcohol, carboxymethyl cellulose, polyethylene glycol, and combinations thereof, but it is not limited thereto.

In one embodiment, examples of the first dispersing agent may comprise, but are not limited to, alginate, gelatin, collagen, demineralized bone matrix, bone morphogenetic protein, albumin, chitosan, fibrin, polyoxyethylene, polyvinylpyrrolidone and a combination thereof while examples of the second dispersing agent may comprise, but are not limited to, hyaluronic acid, polyvinyl alcohol, carboxymethyl cellulose, polyethylene glycol, or a combination thereof.

In one embodiment, the first dispersing agent may be gelatin. The molecular weight of the gelatin is about 10,000-200,000, but it is not limited thereto.

Moreover, in one embodiment, the second dispersing agent may be hyaluronic acid, and the molecular weight of the hyaluronic acid is about 500,000-5,000,000, but it is not limited thereto. In another embodiment, the second dispersing agent may be a combination of hyaluronic acid and polyvinyl alcohol, and the molecular weight of the hyaluronic acid is about 500,000-5,000,000, but it is not limited thereto, and the molecular weight of the polyvinyl alcohol is about 2,000-400,000, but it is not limited thereto. In this embodiment, the weight ratio of the hyaluronic acid to the polyvinyl alcohol is about 1:0.5-5, such as 1:1, 1:2, but it is not limited thereto. In yet another embodiment, the second dispersing agent may be a combination of carboxymethyl cellulose and polyethylene glycol, and the molecular weight of the carboxymethyl cellulose is about 10,000-300,000, but it is not limited thereto, and the molecular weight of the polyethylene glycol is about 2,000-400,000, but it is not limited thereto. In this embodiment, the weight ratio of the carboxymethyl cellulose to polyethylene glycol is about 1:0.1-30, such as 1:15, but it is not limited thereto.

In addition, in the method for method for manufacturing a double layer film of the present disclosure, the solvent of the first hydrophobic solution and the solvent of the second hydrophobic solution are the same. In one specific embodiment, the solvent of the first hydrophobic solution and the solvent of the second hydrophobic solution are dichloromethane.

After the preparation of the first polymer mixture and the second polymer mixture, the first polymer mixture is dried to form a film to form an attachment layer. A manner for drying the first polymer mixture has no particular limitation, only that the first polymer mixture is able to form a form of film. In one embodiment, the first polymer mixture may be poured onto a plate, and then scraped with a scraper to perform a film scraping procedure, and after that, dried to form a film.

Then, after the first polymer mixture is dried to form a film, the second polymer mixture is dried on the aforementioned attachment layer to form a film to form an anti-adhesion layer to complete the preparation of the double layer film. Since the solvent of the first hydrophobic solution existing in the first polymer mixture and the solvent of the second hydrophobic solution existing in the second polymer mixture are the same, when the second polymer mixture is poured onto the attachment layer formed by the first polymer mixture, it will results in that the ingredients at the surface of the attachment layer are slightly dissolved and partially mixed with the attachment layer formed by the second polymer mixture to allow the anti-adhesion layer formed by the second polymer mixture tightly bond to the attachment layer after drying without using an additional binding agent.

In addition, a manner for drying the second polymer mixture has no particular limitation, only that the second polymer mixture is able to form a form of film. In one embodiment, the second polymer mixture may be poured onto the foregoing attachment layer, and then scraped with a scraper to perform a film scraping procedure, and after that, dried to form the anti-adhesion film.

In one embodiment, any double layer film of the present disclosure mentioned above may have a thickness of about 1-3000 μm, and can be curled and efficaciously applied to a minimally invasive surgery. Moreover, in one embodiment, the full roughness (Rz) of any double layer film of the present disclosure mentioned above may be about 1-100 μm.

Furthermore, the burst pressure of any double layer film of the present disclosure mentioned above may be about 5-1000 cm-H₂O.

In addition, the tensile strength of any double layer film of the present disclosure mentioned above may be about 5-3000 kPa.

The suture pullout strength of any double layer film of the present disclosure mentioned above may be about 1-5 N.

The tear resistance of any double layer film of the present disclosure mentioned above may be about 1-5 N.

Furthermore, any double layer film of the present disclosure mentioned above has extremely excellent anti-adhesion, and it can apply to a surgical treatment or a wound for more than 14 days, such as one month without occurrence of adhesion.

Any film of the present disclosure mentioned above is biodegradable, and can be used for reinforcing sutures and preventing leakage in a surgical wound, and can laminate to a tissue by itself without the need of a suture.

Any film of the present disclosure mentioned above can have an isolating effect by attachment while being implanted to a soft tissue or an organ in a common surgery. In addition, any film of the present disclosure mentioned above is capable of preventing exudation of tissue fluid and reinforcing a frail part of a soft tissue. For example, it can be used for patching in a cardiovascular surgery, for patching in a liver, gallbladder, gastrointestinal endoscopy or patching other organs or anadesma without being fixed with surgical sutures.

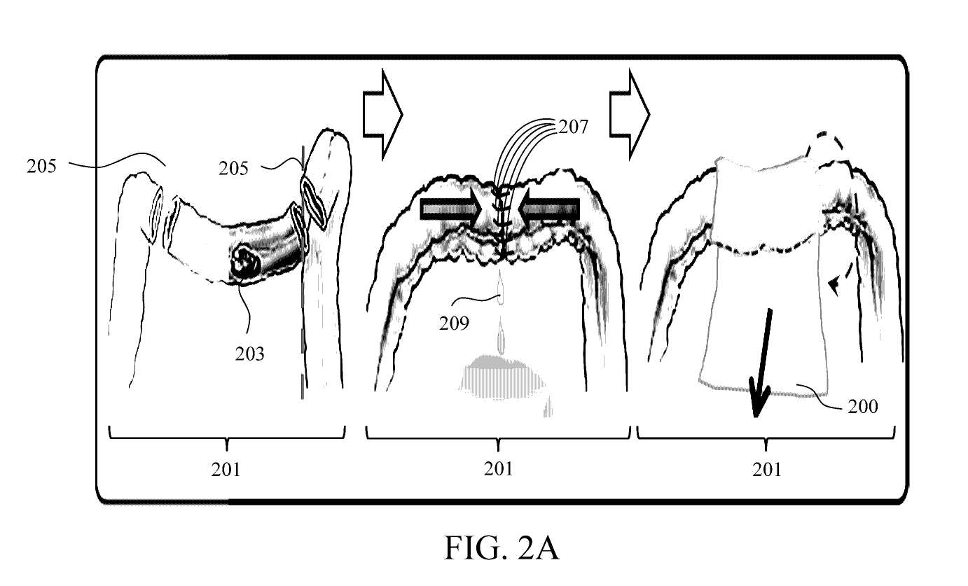

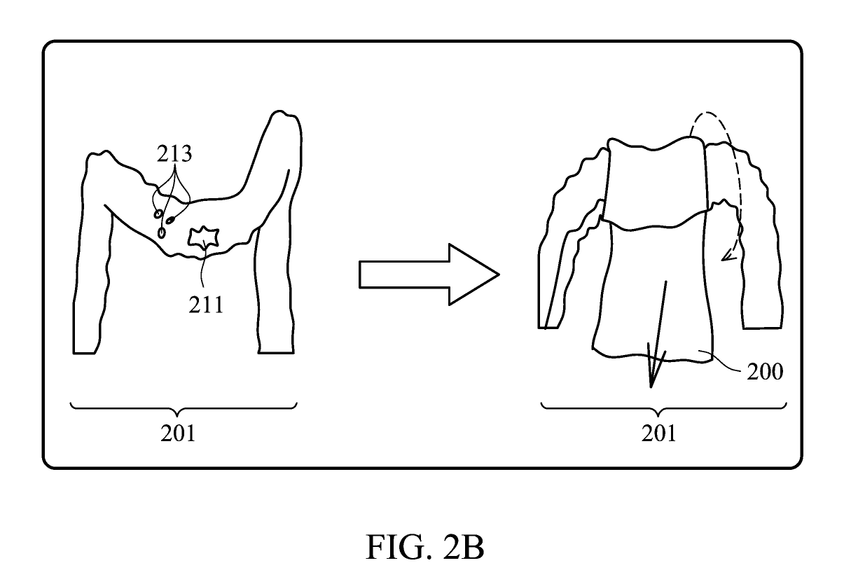

In one embodiment, a situation in which any film of the present disclosure mentioned above may be used is shown as FIG. 2A. A section containing a lesion 203 of a large intestine 201 is excised through excising lines 205. After that, two ends at the excising section of the remaining large intestine are sutured by sutures 207, however, leakage 209 may still occur at the suturing position. Therefore, the suturing position is attached or twined round by a film or double layer film of the present disclosure to achieve the effect of preventing leakage.

In another embodiment, a situation in which any film of the present disclosure mentioned above may be used is shown as FIG. 2B. A large intestine 201 contains a wound 211 or a perforation 213 and thus leakage may occur. The wound or perforation is attached or twined round by a film or double layer film of the present disclosure to achieve the effect of preventing leakage.

Therefore, in another aspect of the present disclosure, a method for sealing a surgical wound or a diffuse wound is provided. The method for sealing a surgical wound or a diffuse wound mentioned above may comprise, but is not limited to, attaching any film of the present disclosure mentioned above to a surgical wound or a diffuse wound of a subject to seal the surgical wound or the diffuse wound. Any film of the present disclosure mentioned above can attach to tissue by itself without needing to be fixed with an external force. In one embodiment, the method for sealing a surgical wound or a diffuse wound of the present disclosed is used in a surgery, but it is not limited thereto.

In any of the aforementioned methods for sealing a surgical wound or a diffuse wound of the present disclosure, the subject may include a vertebrate, etc. The vertebrate mentioned above may comprise a fish, an amphibian, a reptilian, a bird, or a mammal, but it is not limited thereto. Examples of the mammal include, but are not limited to, a human, an orangutan, a monkey, a horse, a donkey, a dog, a cat, a rabbit, a guinea pig, a rat, and a mouse. In one embodiment, in any of the aforementioned methods for sealing a surgical wound or a diffuse wound of the present disclosure, the subject is a human.

### EXAMPLES

A. Preparation of Films

### 1. Comparative Example

### Comparative Example 1: Preparation of Polycaprolactone/Poly(Lactic-Co-Glycolic Acid) (PCL/PLGA) Film

1. 3.2±0.05 g of polycaprolactone (Mw. 120K) was added to 10 ml of dichloromethane (DCM), and then mixed at 50 rpm for 3 hours to form a polycaprolactone solution.

2. 0.8±0.05 g of poly(lactic-co-glycolic acid) (PLGA) (Mw. 240K) was added to 10 ml of dichloromethane (DCM), and then mixed at 50 rpm for 3 hours to form a poly(lactic-co-glycolic acid) solution.

3. The polycaprolactone solution and the poly(lactic-co-glycolic acid) solution were equal in proportion and mixed to form a mixture and continuously stirred.

4. After stirring for about 1 minute±10 seconds, the mixture was poured onto a Teflon plate, and scraped with a 300 μm scraper to perform a film scraping procedure, and after that, left to stand in a fume hood overnight to form a film.

5. The film was removed from the Teflon plate to complete the preparation of a polycaprolactone/poly(lactic-co-glycolic acid)(PCL/PLGA) film.

### 2. Examples

### Example 1: Preparation of Polycaprolactone/Poly(Lactic-Co-Glycolic Acid)/Alginate (PCL/PLGA/AA) Films

### Example 1-1: Preparation of a Film in which the Ratio of (Polycaprolactone/Poly(Lactic-Co-Glycolic Acid)) to Alginate was 1:0.0625

1. 3.2±0.05 g of polycaprolactone (Mw. 120K) was added to 10 ml of dichloromethane (DCM), and then mixed at 50 rpm for 3 hours to form a polycaprolactone solution.

2. 0.8±0.05 g of poly(lactic-co-glycolic acid) (PLGA) (Mw. 240K) was added to 10 ml of dichloromethane (DCM), and then mixed at 50 rpm for 3 hours to form a poly(lactic-co-glycolic acid) solution.

3. The polycaprolactone solution and the poly(lactic-co-glycolic acid) solution were uniformly mixed to form a mixture solution.

4. 0.25 g of alginate (AA) was added to the mixture solution to form a mixture and continuously stirred.

5. After stirring for about 1 minute±10 seconds, the mixture was poured onto a Teflon plate, and scraped with a 300 μm scraper to perform a film scraping procedure, and after that, left to stand in a fume hood overnight to form a film.

6. The film was removed from the Teflon plate.

7. The film was washed 4 times with 2 L deionized water for 1 hour.

8. After washing, the film was placed in a 37° C. oven for drying for 16-24 hours to complete the preparation of polycaprolactone/poly(lactic-co-glycolic acid)/alginate (PCL/PLGA/AA) film of Example 1-1.

### Example 1-2: Preparation of a Film in which the Ratio of (Polycaprolactone/Poly(Lactic-Co-Glycolic Acid)) to Alginate was 1:0.625

1. 3.2±0.05 g of polycaprolactone (Mw. 120K) was added to 10 ml of dichloromethane (DCM), and then mixed at 50 rpm for 3 hours to form a polycaprolactone solution.

2. 0.8±0.05 g of poly(lactic-co-glycolic acid) (PLGA) (Mw. 240K) was added to 10 ml of dichloromethane (DCM), and then mixed at 50 rpm for 3 hours to form a poly(lactic-co-glycolic acid) solution.

3. The polycaprolactone solution and the poly(lactic-co-glycolic acid) solution were uniformly mixed to form a mixture solution.

4. 2.5 g of alginate (AA) was added to the mixture solution to form a mixture and continuously stirred.

5. After stirring for about 1 minute±10 seconds, the mixture was poured onto a Teflon plate, and scraped with a 300 μm scraper to perform a film scraping procedure, and after that, left to stand in a fume hood overnight to form a film.

6. The film was removed from the Teflon plate.

7. The film was washed 4 times with 2 L deionized water for 1 hour.

8. After washing, the film was placed in a 37° C. oven for drying for 16-24 hours to complete the preparation of polycaprolactone/poly(lactic-co-glycolic acid)/alginate (PCL/PLGA/AA) film of Example 1-2.

### Example 1-3: Preparation of a Film in which the Ratio of (Polycaprolactone/Poly(Lactic-Co-Glycolic Acid)) to Alginate was 1:20

1. 3.2±0.05 g of polycaprolactone (Mw. 120K) was added to 10 ml of dichloromethane (DCM), and then mixed at 50 rpm for 3 hours to form a polycaprolactone solution.

2. 0.8±0.05 g of poly(lactic-co-glycolic acid) (PLGA) (Mw. 240K) was added to 10 ml of dichloromethane (DCM), and then mixed at 50 rpm for 3 hours to form a poly(lactic-co-glycolic acid) solution.

3. The polycaprolactone solution and the poly(lactic-co-glycolic acid) solution were uniformly mixed to form a mixture solution.

4. 100 g of alginate (AA) was added to the mixture solution to form a mixture and continuously stirred.

5. After stirring for about 1 minute±10 seconds, the mixture was poured onto a Teflon plate, and scraped with a 300 μm scraper to perform a film scraping procedure, and after that, left to stand in a fume hood overnight to form a film.

6. The film was removed from the Teflon plate.

7. The film was washed 4 times with 2 L deionized water for 1 hour.

8. After washing, the film was placed in a 37° C. oven for drying for 16-24 hours to complete the preparation of polycaprolactone/poly(lactic-co-glycolic acid)/alginate (PCL/PLGA/AA) film of Example 1-3.

### Example 1-4: Preparation of a Film in which the Ratio of (Polycaprolactone/Poly(Lactic-Co-Glycolic Acid)) to Alginate was 1:60

1. 3.2±0.05 g of polycaprolactone (Mw. 120K) was added to 10 ml of dichloromethane (DCM), and then mixed at 50 rpm for 3 hours to form a polycaprolactone solution.

2. 0.8±0.05 g of poly(lactic-co-glycolic acid) (PLGA) (Mw. 240K) was added to 10 ml of dichloromethane (DCM), and then mixed at 50 rpm for 3 hours to form a poly(lactic-co-glycolic acid) solution.

3. The polycaprolactone solution and the poly(lactic-co-glycolic acid) solution were uniformly mixed to form a mixture solution.

4. 240 g of alginate (AA) was added to the mixture solution to form a mixture and continuously stirred.

5. After stirring for about 1 minute±10 seconds, the mixture was poured onto a Teflon plate, and scraped with a 300 μm scraper to perform a film scraping procedure, and after that, left to stand in a fume hood overnight to form a film.

6. The film was removed from the Teflon plate.

7. The film was washed 4 times with 2 L deionized water for 1 hour.

8. After washing, the film was placed in a 37° C. oven for drying for 16-24 hours to complete the preparation of polycaprolactone/poly(lactic-co-glycolic acid)/alginate (PCL/PLGA/AA) film.

### Example 2: Preparation of Polycaprolactone/Gelatin (PCL/Gelatin) Film

### Example 2-1: Preparation of a Film in which the Ratio of Polycaprolactone to Gelatin was 1:1.25

1. 4 g of polycaprolactone (Mw. 120K) was added to 20 ml of dichloromethane (DCM), and then mixed at 50 rpm for 3 hours to prepare a 20% polycaprolactone solution.

2. 5 g of gelatin was added to 10 ml of deionized water and heated in a 50° C. oven for 16 hours to be dissolved to prepare a 50% gelatin solution.

3. The 50% gelatin solution was removed from the oven and poured into the 20% polycaprolactone solution (time for taking the 50% gelatin solution out and pouring it to the 20% polycaprolactone solution had to be in 1 minute) to perform mixing and stirring to form a mixture.

4. After stirring for about 1 minute±10 seconds, the mixture was poured onto a Teflon plate, and scraped with a 300 μm scraper to perform a film scraping procedure, and after that, left to stand in a fume hood overnight to form a film.

5. The film was removed from the Teflon plate to complete the preparation of polycaprolactone/gelatin (PCL/Gelatin) film.

### Example 2-2: Preparation of a Film in which the Ratio of Polycaprolactone to Gelatin was 1:0.925

1. 4 g of polycaprolactone (Mw. 120K) was added to 20 ml of dichloromethane (DCM), and dissolved at a room temperature with a dissolving time of 16-24 hours to prepare a 20 wt % polycaprolactone solution.

2. 3.7 g of gelatin was added to 10 ml of deionized water and heated in a 50° C. oven for 16-24 hours to be dissolved to prepare a 37% gelatin solution.

3. The 37% gelatin solution was removed from the oven and poured into the 20 wt % polycaprolactone solution at a 2:1 (v/v) blending ratio of gelatin solution to polycaprolactone solution and blended at a stirring rate of about 45-80 rpm to form a mixture (viscosity: 582 CP) while the blending needed to be complete in 45-90 seconds.

4. After uniformly blending, the aforementioned mixture was poured onto a smooth glass plate or Teflon plate, and scraped by a film scraping machine having a 150 μm scraper at a scraping rate of 35 mm/s to perform a film scraping procedure, and after that, left to stand in a fume hood for 16-24 hours to volatilize the solvent to form a film.

5. The film was removed from the smooth glass plate or Teflon plate to complete the preparation of polycaprolactone/gelatin (PCL/Gelatin) film.

### Example 2-3: Preparation of a Film in which the Ratio of Polycaprolactone to Gelatin was 1:0.74

1. 5 g of polycaprolactone (Mw. 120K) was added to 20 ml of dichloromethane (DCM), and dissolved at a room temperature with a dissolving time of 16-24 hours to prepare a 25 wt % polycaprolactone solution.

2. 3.7 g of gelatin was added to 10 ml of deionized water and heated in a 50° C. oven for 16-24 hours to be dissolved to prepare a 37% gelatin solution.

3. The 37% gelatin solution was removed from the oven and poured into the 25 wt % polycaprolactone solution at a 2:1 (v/v) blending ratio of gelatin solution to polycaprolactone solution and blended at a stirring rate of about 45-80 rpm to form a mixture while the blending needed to be complete in 45-90 seconds.

4. After uniformly blending, the aforementioned mixture was poured onto a smooth glass plate or Teflon plate, and scraped by a film scraping machine having a 150 μm scraper at a scraping rate of 35 mm/s to perform a film scraping procedure, and after that, left to stand in a fume hood for 16-24 hours to volatilize the solvent to form a film.

5. The film was removed from the smooth glass plate or Teflon plate to complete the preparation of polycaprolactone/gelatin (PCL/Gelatin) film.

### Example 2-4: Preparation of a Film in which the Ratio of Polycaprolactone to Gelatin was 1:0.6167

1. 6 g of polycaprolactone (Mw. 120K) was added to 20 ml of dichloromethane (DCM), and dissolved at a room temperature with a dissolving time of 16-24 hours to prepare a 30 wt % polycaprolactone solution.

2. 3.7 g of gelatin was added to 10 ml of deionized water and heated in a 50° C. oven for 16-24 hours to be dissolved to prepare a 37% gelatin solution.

3. The 37% gelatin solution was removed from the oven and poured into the 30 wt % polycaprolactone solution at a 2:1 (v/v) blending ratio of gelatin solution to polycaprolactone solution and blended at a stirring rate of about 45-80 rpm to form a mixture while the blending needed to be complete in 45-90 seconds.

4. After uniformly blending, the aforementioned mixture was poured onto a smooth glass plate or Teflon plate, and scraped by a film scraping machine having a 150 μm scraper at a scraping rate of 35 mm/s to perform a film scraping procedure, and after that, left to stand in a fume hood for 16-24 hours to volatilize the solvent to form a film.

5. The film was removed from the smooth glass plate or Teflon plate to complete the preparation of polycaprolactone/gelatin (PCL/Gelatin) film.

### Example 3: Preparation of Polycaprolactone/Poly(Lactic-Co-Glycolic Acid)/Gelatin (PCL/PLGA/Gelatin) Film

### Example 3-1: Preparation of a Film in which the Ratio of Polycaprolactone/Poly(Lactic-Co-Glycolic Acid) to Gelatin was 1:1.25

1. 3.2 g of polycaprolactone (Mw. 120K) and 0.8 g of poly(lactic-co-glycolic acid) were added to 20 ml of dichloromethane (DCM), and then mixed at 50 rpm for 3 hours to prepare a polycaprolactone/poly(lactic-co-glycolic acid) solution.

2. 5 g of gelatin was added to 10 ml of deionized water and heated in a 50° C. oven for 16 hours to be dissolved to prepare a 50% gelatin solution.

3. The 50% gelatin solution was removed from the oven and poured into the 20% polycaprolactone/poly(lactic-co-glycolic acid) solution (time for taking the 50% gelatin solution out and pouring it to the polycaprolactone solution has to be in 1 minute) to perform mixing and stirring to form a mixture.

4. After stirring for about 1 minute±10 seconds, the mixture was poured onto a Teflon plate, and scraped with a 300 μm scraper to perform a film scraping procedure, and after that, left to stand in a fume hood overnight to form a film.

5. The film was removed from the Teflon plate to complete the preparation of polycaprolactone/poly(lactic-co-glycolic acid)/gelatin (PCL/PLGA/Gelatin) film.

### Example 3-2: Preparation of a Film in which the Ratio of Polycaprolactone/Poly(Lactic-Co-Glycolic Acid) to Gelatin was 1:0.925

1. 3.2 g of polycaprolactone (Mw. 120K) and 0.8 g of poly(lactic-co-glycolic acid) were added to 20 ml of dichloromethane (DCM), and dissolved at a room temperature with a dissolving time of 16-24 hours to prepare a polycaprolactone/poly(lactic-co-glycolic acid) solution.

2. 3.7 g of gelatin was added to 10 ml of deionized water and heated in a 50° C. oven for 16-24 hours to be dissolved to prepare a 37% gelatin solution.

3. The 37% gelatin solution was removed from the oven and poured into the polycaprolactone/poly(lactic-co-glycolic acid) solution at a 2:1 (v/v) blending ratio of gelatin solution to polycaprolactone solution and blended at a stirring rate of about 45-80 rpm to form a mixture while the blending needed to be complete in 45-90 seconds.

4. After uniformly blending, the aforementioned mixture was poured onto a smooth glass plate or Teflon plate, and scraped by a film scraping machine having a 150 μm scraper at a scraping rate of 35 mm/s to perform a film scraping procedure, and after that, left to stand in a fume hood for 16-24 hours to volatilize the solvent to form a film.

5. The film was removed from the smooth glass plate or Teflon plate to complete the preparation of polycaprolactone/poly(lactic-co-glycolic acid)/gelatin (PCL/PLGA/Gelatin) film.

### Example 4: Preparation of Polycaprolactone/Hyaluronic Acid (PCL/Hyaluronic Acid) Film

### Example 4-1: Preparation of a Film in which the Ratio of Polycaprolactone to Hyaluronic Acid was 1:0.025

1. 4 g of polycaprolactone (Mw. 120K) was added to 20 ml of dichloromethane (DCM), and dissolved at a room temperature with a dissolving time of 16-24 hours to prepare a 20 wt % polycaprolactone solution.

2. 0.1 g of hyaluronic acid was added to 10 ml of deionized water and heated in a 50° C. oven for 16-24 hours to be dissolved to prepare a 1 wt % hyaluronic acid solution.

3. The 1 wt % hyaluronic acid solution was removed from the oven and poured into the 20 wt % polycaprolactone solution at a 2:1 (v/v) blending ratio of hyaluronic acid solution to polycaprolactone solution and blended at a stirring rate of about 45-80 rpm to form a mixture while the blending needed to be complete in 45-90 seconds.

4. After uniformly blending, the aforementioned mixture was poured onto a smooth glass plate or Teflon plate, and scraped by a film scraping machine having a 150 μm scraper at a scraping rate of 35 mm/s to perform a film scraping procedure, and after that, left to stand in a fume hood for 16-24 hours to volatilize the solvent to form a film.

5. The film was removed from the smooth glass plate or Teflon plate to complete the preparation of polycaprolactone/hyaluronic acid (PCL/HA) film.

### Example 4-2: Preparation of a Film in which the Ratio of Polycaprolactone to Hyaluronic Acid was 1:0.02

1. 5 g of polycaprolactone (Mw. 120K) was added to 20 ml of dichloromethane (DCM), and dissolved at a room temperature with a dissolving time of 16-24 hours to prepare a 25 wt % polycaprolactone solution.

2. 0.1 g of hyaluronic acid was added to 10 ml of deionized water and heated in a 50° C. oven for 16-24 hours to be dissolved to prepare a 1 wt % hyaluronic acid solution.

3. The 1 wt % hyaluronic acid solution was removed from the oven and poured into the 25 wt % polycaprolactone solution at a 2:1 (v/v) blending ratio of hyaluronic acid solution to polycaprolactone solution and blended at a stirring rate of about 45-80 rpm to form a mixture while the blending needed to be complete in 45-90 seconds.

4. After uniformly blending, the aforementioned mixture was poured onto a smooth glass plate or Teflon plate, and scraped by a film scraping machine having a 150 μm scraper at a scraping rate of 35 mm/s to perform a film scraping procedure, and after that, left to stand in a fume hood for 16-24 hours to volatilize the solvent to form a film.

5. The film was removed from the smooth glass plate or Teflon plate to complete the preparation of polycaprolactone/hyaluronic acid (PCL/HA) film.

### Example 4-3: Preparation of a Film in which the Ratio of Polycaprolactone to Hyaluronic Acid was 1:0.0167

1. 6 g of polycaprolactone (Mw. 120K) was added to 20 ml of dichloromethane (DCM), and dissolved at a room temperature with a dissolving time of 16-24 hours to prepare a 30 wt % polycaprolactone solution.

2. 0.1 g of hyaluronic acid was added to 10 ml of deionized water and heated in a 50° C. oven for 16-24 hours to be dissolved to prepare a 1 wt % hyaluronic acid solution.

3. The 1 wt % hyaluronic acid solution was removed from the oven and poured into the 30 wt % polycaprolactone solution at a 2:1 (v/v) blending ratio of hyaluronic acid solution to polycaprolactone solution and blended at a stirring rate of about 45-80 rpm to form a mixture while the blending needed to be complete in 45-90 seconds.

4. After uniformly blending, the aforementioned mixture was poured onto a smooth glass plate or Teflon plate, and scraped by a film scraping machine having a 150 μm scraper at a scraping rate of 35 mm/s to perform a film scraping procedure, and after that, left to stand in a fume hood for 16-24 hours to volatilize the solvent to form a film.

5. The film was removed from the smooth glass plate or Teflon plate to complete the preparation of polycaprolactone/hyaluronic acid (PCL/HA) film.

### Example 5

### Example 5-1: Preparation of a Film in which the Ratio of Polycaprolactone to (Hyaluronic Acid/Polyvinyl Alcohol) was 1:0.05

1. 4 g of polycaprolactone (Mw. 120K) was added to 20 ml of dichloromethane (DCM), and dissolved at a room temperature with a dissolving time of 16-24 hours to prepare a 20 wt % polycaprolactone solution.

2. 0.1 g of hyaluronic acid and 0.1 g of polyvinyl alcohol were added to 10 ml of deionized water and heated in a 50° C. oven for 16-24 hours to be dissolved to prepare a hyaluronic acid/polyvinyl alcohol solution.

3. The hyaluronic acid/polyvinyl alcohol solution was removed from the oven and poured into the 20 wt % polycaprolactone solution at a 2:1 (v/v) blending ratio of hyaluronic acid/polyvinyl alcohol solution to polycaprolactone solution and blended at a stirring rate of about 45-80 rpm to form a mixture (viscosity: 402 CP) while the blending needed to be complete in 45-90 seconds.

4. After uniformly blending, the aforementioned mixture was poured onto a smooth glass plate or Teflon plate, and scraped by a film scraping machine having a 150 μm scraper at a scraping rate of 35 mm/s to perform a film scraping procedure, and after that, left to stand in a fume hood for 16-24 hours to volatilize the solvent to form a film.

5. The film was removed from the smooth glass plate or Teflon plate to complete the preparation of polycaprolactone/hyaluronic acid/polyvinyl alcohol (PCL/HA/PVA) film.

### Example 5-2: Preparation of a Film in which the Ratio of Polycaprolactone to (Hyaluronic Acid/Polyvinyl Alcohol) was 1:0.075

1. 4 g of polycaprolactone (Mw. 120K) was added to 20 ml of dichloromethane (DCM), and dissolved at a room temperature with a dissolving time of 16-24 hours to prepare a 20 wt % polycaprolactone solution.

2. 0.1 g of hyaluronic acid and 0.2 g of polyvinyl alcohol were added to 10 ml of deionized water and heated in a 50° C. oven for 16-24 hours to be dissolved to prepare a hyaluronic acid/polyvinyl alcohol solution.

3. The hyaluronic acid/polyvinyl alcohol solution was removed from the oven and poured into the 20 wt % polycaprolactone solution at a 2:1 (v/v) blending ratio of hyaluronic acid/polyvinyl alcohol solution to polycaprolactone solution and blended at a stirring rate of about 45-80 rpm to form a mixture while the blending needed to be complete in 45-90 seconds.

4. After uniformly blending, the aforementioned mixture was poured onto a smooth glass plate or Teflon plate, and scraped by a film scraping machine having a 150 μm scraper at a scraping rate of 35 mm/s to perform a film scraping procedure, and after that, left to stand in a fume hood for 16-24 hours to volatilize the solvent to form a film.

5. The film was removed from the smooth glass plate or Teflon plate to complete the preparation of polycaprolactone/hyaluronic acid/polyvinyl alcohol (PCL/HA/PVA) film.

### Example 6: Preparation of a Film in which the Ratio of Polycaprolactone to (Carboxymethyl Cellulose/Polyethylene Glycol) was 1:0.8

1. 4 g of polycaprolactone (Mw. 120K) was added to 20 ml of dichloromethane (DCM), and dissolved at a room temperature with a dissolving time of 16-24 hours to prepare a 20 wt % polycaprolactone solution.

2. 0.2 g of carboxymethyl cellulose and 3 g of polyethylene glycol were added to 10 ml of deionized water and heated in a 50° C. oven for 16-24 hours to be dissolved to prepare a carboxymethyl cellulose/polyethylene glycol solution.

3. The carboxymethyl cellulose/polyethylene glycol solution was removed from the oven and poured into the 20 wt % polycaprolactone solution at a 2:1 (v/v) blending ratio of carboxymethyl cellulose/polyethylene glycol solution to polycaprolactone solution and blended at a stirring rate of about 45-80 rpm to form a mixture while the blending needed to be complete in 45-90 seconds.

4. After uniformly blending, the aforementioned mixture was poured onto a smooth glass plate or Teflon plate, and scraped by a film scraping machine having a 150 μm scraper at a scraping rate of 35 mm/s to perform a film scraping procedure, and after that, left to stand in a fume hood for 16-24 hours to volatilize the solvent to form a film.

5. The film was removed from the smooth glass plate or Teflon plate to complete the preparation of polycaprolactone/carboxymethyl cellulose/polyethylene glycol (PCL/CMC/PEGA) film.

### Example 7: Preparation of Double Layer Films

### Example 7-1: Preparation of a Double Layer (PCL/Gelatin (1:0.925) Film-PCL/HA (1:0.025) Film)

1. After the mixture of step 3 of Example 2-2 was uniformly blended, the mixture (viscosity: 582 CP) was poured onto a smooth glass plate or Teflon plate, and scraped by a film scraping machine having a 150 μm scraper at a scraping rate of 35 mm/s to perform a film scraping procedure, and after that, left to stand in a fume hood for 20-30 minutes to volatilize the solvent to form a first layer of film (an attachment layer).

2. After the mixture of step 3 of Example 4-1 was uniformly blended, the mixture (viscosity: 348 CP) was poured onto the first layer of film, and scraped by a film scraping machine having a 150 μm scraper at a scraping rate of 35 mm/s to perform a film scraping procedure to perform a forming procedure for a second layer of film (anti-adhesion layer), and after that, left to stand in a fume hood for 16-24 hours to volatilize the solvent to obtain a double layer film (PCL/Gelatin (1:0.925) film-PCL/HA (1:0.025) film).

### Example 7-2: Preparation of a Double Layer (PCL/Gelatin (1:0.74) Film-PCL/HA (1:0.02) Film)

1. After the mixture of step 3 of Example 2-3 was uniformly blended, the mixture was poured onto a smooth glass plate or Teflon plate, and scraped by a film scraping machine having a 150 μm scraper at a scraping rate of 35 mm/s to perform a film scraping procedure, and after that, left to stand in a fume hood for 20-30 minutes to volatilize the solvent to form a first layer of film (an attachment layer).

2. After the mixture of step 3 of Example 4-2 was uniformly blended, the mixture was poured onto the first layer of film, and scraped by a film scraping machine having a 150 μm scraper at a scraping rate of 35 mm/s to perform a film scraping procedure to perform a forming procedure for a second layer of film (anti-adhesion layer), and after that, left to stand in a fume hood for 16-24 hours to volatilize the solvent to obtain a double layer film (PCL/Gelatin (1:0.74) film-PCL/HA (1:0.02) film).

### Example 7-3: Preparation of a Double Layer (PCL/Gelatin (1:0.6167) Film-PCL/HA (1:0.0167) Film)

1. After the mixture of step 3 of Example 2-4 was uniformly blended, the mixture was poured onto a smooth glass plate or Teflon plate, and scraped by a film scraping machine having a 150 μm scraper at a scraping rate of 35 mm/s to perform a film scraping procedure, and after that, left to stand in a fume hood for 20-30 minutes to volatilize the solvent to form a first layer of film (an attachment layer).

2. After the mixture of step 3 of Example 4-3 was uniformly blended, the mixture was poured onto the first layer of film, and scraped by a film scraping machine having a 150 μm scraper at a scraping rate of 35 mm/s to perform a film scraping procedure to perform a forming procedure for a second layer of film (anti-adhesion layer), and after that, left to stand in a fume hood for 16-24 hours to volatilize the solvent to obtain a double layer film (PCL/Gelatin (1:0.6167) film-PCL/HA (1:0.0167) film).

### Example 7-4: Preparation of a Double Layer (PCL/Gelatin (1:0.925) Film-PCL/(HA/PVA) (1:0.05) Film)

1. After the mixture of step 3 of Example 2-2 was uniformly blended, the mixture (viscosity: 582 CP) was poured onto a smooth glass plate or Teflon plate, and scraped by a film scraping machine having a 150 μm scraper at a scraping rate of 35 mm/s to perform a film scraping procedure, and after that, left to stand in a fume hood for 20-30 minutes to volatilize the solvent to form a first layer of film (an attachment layer).

2. After the mixture of step 3 of Example 5-1 was uniformly blended, the mixture (viscosity: 402 CP) was poured onto the first layer of film, and scraped by a film scraping machine having a 150 μm scraper at a scraping rate of 35 mm/s to perform a film scraping procedure to perform a forming procedure for a second layer of film (anti-adhesion layer), and after that, left to stand in a fume hood for 16-24 hours to volatilize the solvent to obtain a double layer film (PCL/Gelatin (1:0.925) film-PCL/(HA/PVA) (1:0.05) film).

### Example 7-5: Preparation of a Double Layer (PCL/Gelatin (1:0.925) Film-PCL/(HA/PVA) (1:0.075) Film)

1. After the mixture of step 3 of Example 2-2 was uniformly blended, the mixture was poured onto a smooth glass plate or Teflon plate, and scraped by a film scraping machine having a 150 μm scraper at a scraping rate of 35 mm/s to perform a film scraping procedure, and after that, left to stand in a fume hood for 20-30 minutes to volatilize the solvent to form a first layer of film (an attachment layer).

2. After the mixture of step 3 of Example 5-2 was uniformly blended, the mixture was poured onto the first layer of film, and scraped by a film scraping machine having a 150 μm scraper at a scraping rate of 35 mm/s to perform a film scraping procedure to perform a forming procedure for a second layer of film (anti-adhesion layer), and after that, left to stand in a fume hood for 16-24 hours to volatilize the solvent to obtain a double layer film (PCL/Gelatin (1:0.925) film-PCL/(HA/PVA) (1:0.075) film).

### Example 7-6: Preparation of a Double Layer (PCL/Gelatin (1:0.925) Film-PCL/(CMC/PEG) (1:0.8) Film)

1. After the mixture of step 3 of Example 2-2 was uniformly blended, the mixture was poured onto a smooth glass plate or Teflon plate, and scraped by a film scraping machine having a 150 μm scraper at a scraping rate of 35 mm/s to perform a film scraping procedure, and after that, left to stand in a fume hood for 20-30 minutes to volatilize the solvent to form a first layer of film (an attachment layer).

2. After the mixture of step 3 of Example 6 was uniformly blended, the mixture was poured onto the first layer of film, and scraped by a film scraping machine having a 150 μm scraper at a scraping rate of 35 mm/s to perform a film scraping procedure to perform a forming procedure for a second layer of film (anti-adhesion layer), and after that, left to stand in a fume hood for 16-24 hours to volatilize the solvent to obtain a double layer film (PCL/Gelatin (1:0.925) film-PCL/(CMC/PEG) (1:0.8) film).

B. Viscosity Analysis of the Hydrophilic Polymer,

Hydrophilic materials, gelatin, hyaluronic acid, polyvinyl alcohol, used in the films were analyzed by a viscometer.

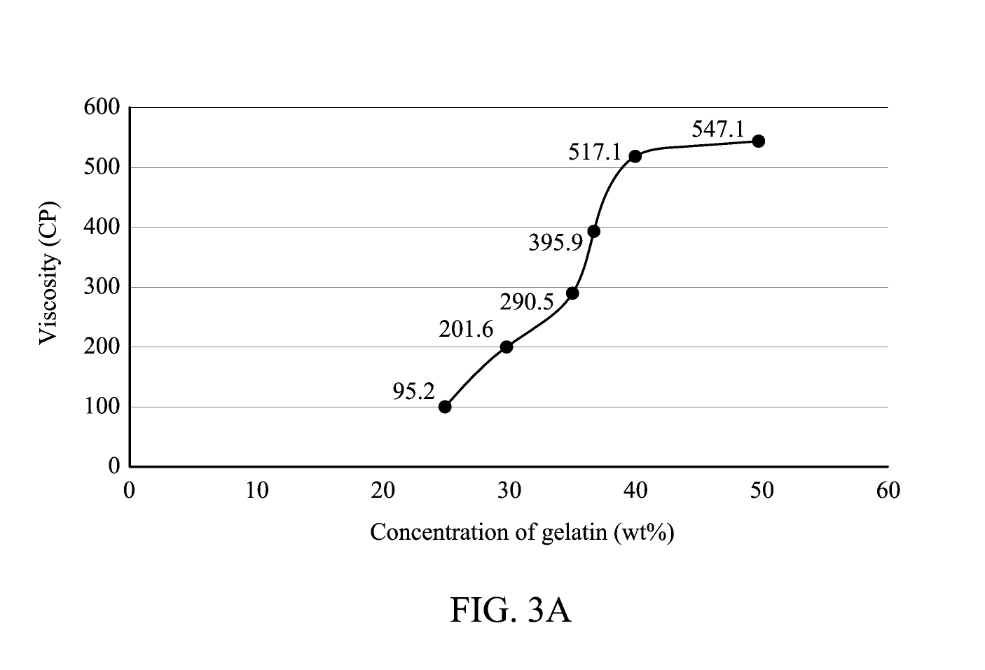

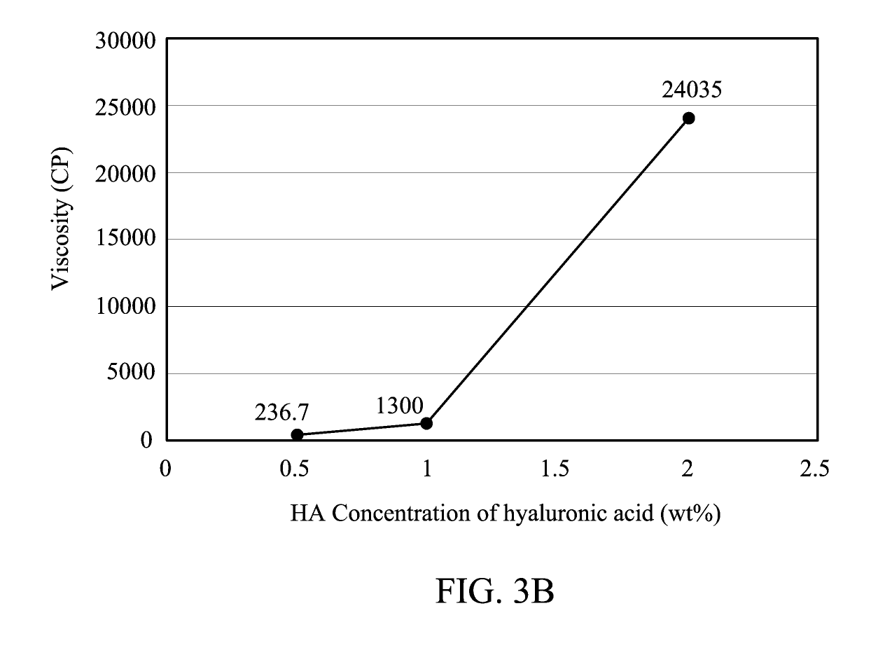

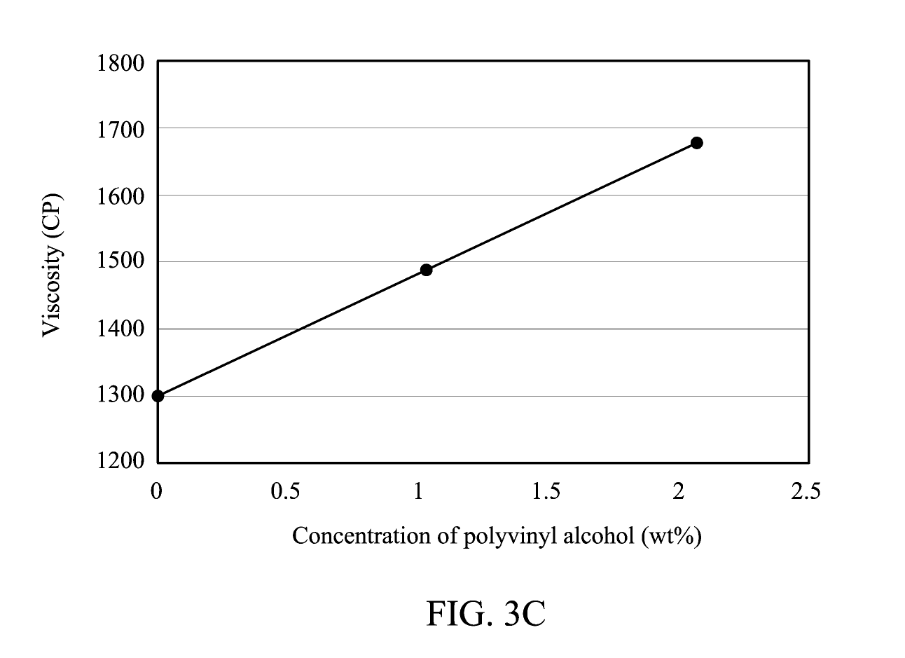

First, hydrophilic materials to be determined were dissolved by d. d. water and prepared according to the volume required by the instrument. Next, the material was poured into the sample tank of the viscometer to perform a temperature equilibration and the temperature was maintained within a range of 50±1° C. by a circulating water bath to keep the temperature constant, wherein pre-stirring was performed during the course of keeping the temperature constant. After maintaining a constant temperature for 30 minutes, Viscosity value recording was started. The results are shown in FIGS. 3A, 3B and 3C.

FIGS. 3A, 3B and 3C show viscosity analysis results for gelatin, hyaluronic acid and polyvinyl alcohol, respectively.

C. Film Property Analysis

1. Uniformity Analysis for Films of Polycaprolactone Blended with Hydrophilic and/or Hydrophobic Polymer

Thermogravimetric Analysis (TGA)

Thermogravimetric analysis is often used to determine the properties of a substance by the decrease or increase in mass resulting from decomposition, oxidation or volatilization (such as volatilization of moisture content). Thermogravimetric analysis can be used to accurately predict material structure, or it can be directly used as a chemical analysis, and as a technique for observing blending uniformity.

The thermogravimetric analyzer used in the present experiment was Pyris 1 TGA.

(1) Thermogravimetric Analysis for the Films Prepared by Examples 1-1 to 1-4, and for the Films Prepared by Comparative Example 1, Example 2-1 and Example 3-1.

The procedure for operation and analysis is described in the following paragraphs.

The machine and computer were turned on, and it was confirmed that the machine was connected to the computer. The gas used was high-purity nitrogen, and it was confirmed that the nitrogen was sufficient and was led into the machine. “Pyris Manager” was clicked, and the thermogravimetric analysis software was started. The determining parameter conditions were set: Initial temperature was 25° C. The temperature was increased to 700° C. at a rate of 20° C. per minute, and then maintained at 700° C. for 15 minutes. Information related to the file was filled out, such as storage location, file name, remarks, etc. According to the operation of the machine, a required platinum plate was hung on a balance of the machine, a button for balancing was clicked to reset the weight of the platinum plate to zero. A temperature controlling barrier was lowered and the platinum plate was taken out and the sample to be tested was placed on the platinum plate, and the weight of the sample was controlled at 3-30 mg. A button for weighing was clicked to weigh the sample, and after the weight was determined, the measurement began. The result of the measurement was saved as an ASC file and analyzed.

First, the influence of the content of the hydrophilic polymer, alginate, on the uniformity of a film of polycaprolactone blended with hydrophilic and/or hydrophobic polymer was determined.

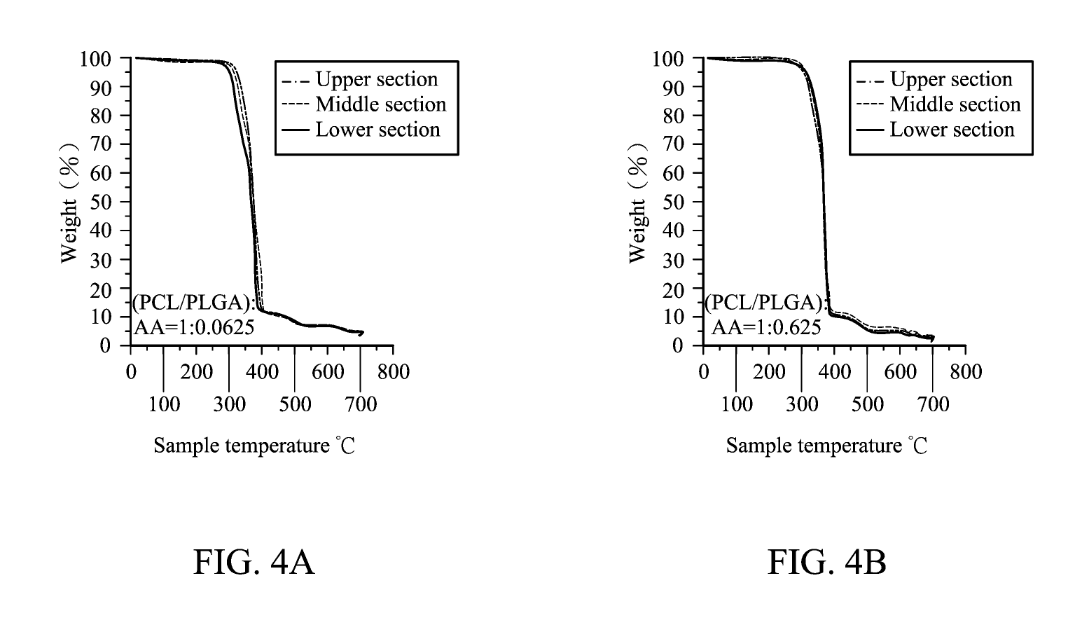

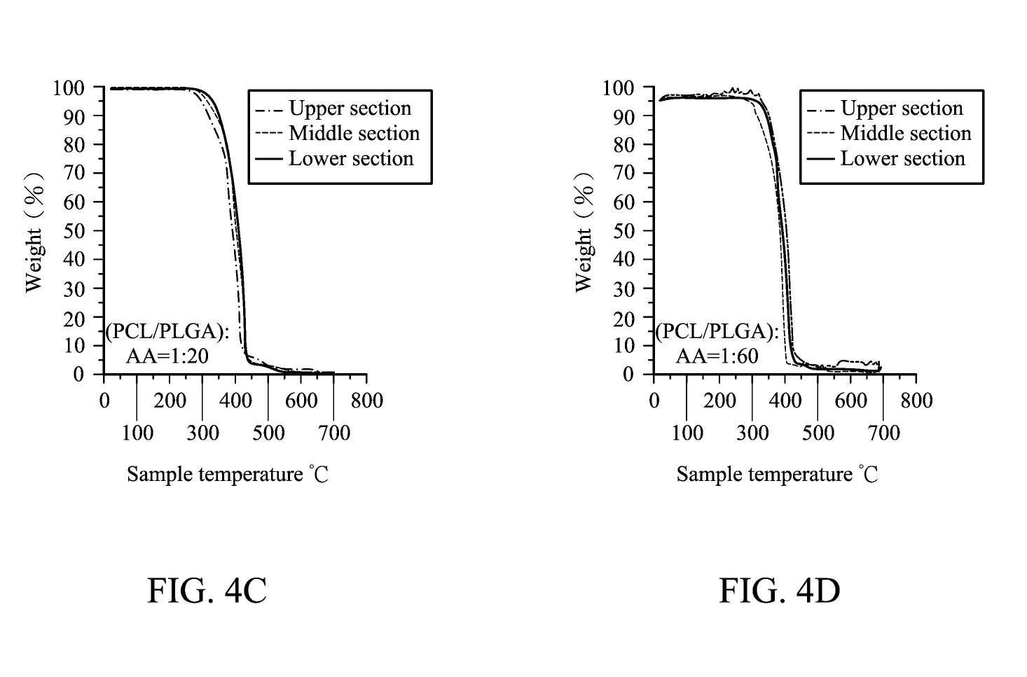

Films formed by blending different weights of alginate to a fixed weight of polycaprolactone/poly(lactic-co-glycolic acid) (the films prepared in Examples 1-1 to 1-4) were observed for composition uniformity at different locations (a film was cross cut into three sections: an upper section, a middle section, and a lower section) using a thermogravimetric analyzer. In the film prepared in Example 1-1, the weight ratio of (polycaprolactone/poly(lactic-co-glycolic acid)) to alginate was 1:0.0625. In the film prepared in Example 1-2, the weight ratio of (polycaprolactone/poly(lactic-co-glycolic acid)) to alginate was 1:0.625. In the film prepared in Example 1-3, the weight ratio of (polycaprolactone/poly(lactic-co-glycolic acid)) to alginate was 1:20. In the film prepared in Example 1-4, the weight ratio of (polycaprolactone/poly(lactic-co-glycolic acid)) to alginate was 1:60. The thermogravimetric analysis results for the films of Examples 1-1 to 1-4 are shown in FIGS. 4A to 4D, respectively.

The thermogravimetric analysis results showed that the films prepared in Example 1-2 was most uniform, i.e. when the weight ratio of the hydrophobic composition to the hydrophilic composition was 1:0.625, a film was most uniformly formed (FIG. 4B).

In addition, it was determined whether a hydrophilic polymer which was different from alginate in a film of polycaprolactone blended with hydrophilic and/or hydrophobic polymer was also capable of achieving the effect of uniformly forming a film.

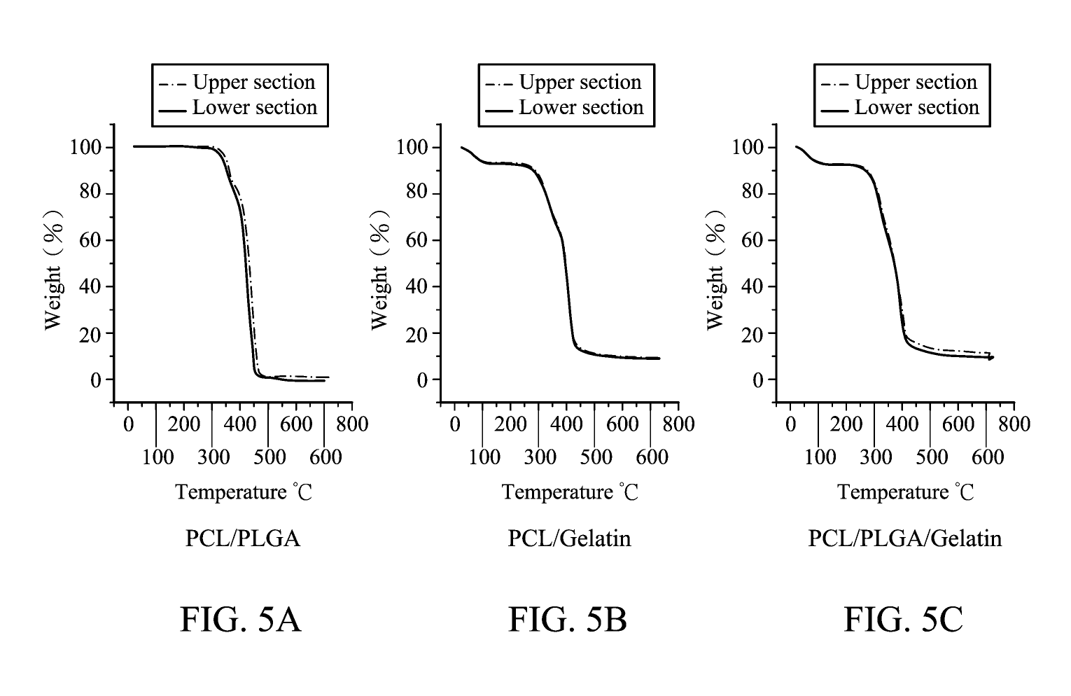

Films formed in Comparative Example 1 (polycaprolactone/poly(lactic-co-glycolic acid) film), Example 2-1 (polycaprolactone/gelatin film) and Example 3-1 (polycaprolactone/poly(lactic-co-glycolic acid)/gelatin) were observed for composition uniformity at different locations (a film was cross cut into two sections: an upper section and a lower section) using a thermogravimetric analyzer. The results are shown in FIGS. 5A to 5C.

FIGS. 5A to 5C show that the film prepared in Example 2-1 shows only one TGA curve while the film prepared in Example 3-1 shows two TGA curves which are completely overlapping. This indicated that by using the hydrophilic polymer, gelatin, as a dispersing agent, polycaprolactone can be uniformly blended with poly(lactic-co-glycolic acid) without phase separation occurring.

In contrast, the film prepared in Comparative Example 1 showed two obvious TGA curves. This indicated that the film formed without using a hydrophilic polymer as a dispersing agent was extremely nonuniform with phase separation occurring.

(2) Thermogravimetric Analysis for the Double Films Prepared by Examples 7-1 to 7-5

The procedure for operation and analysis is described in the following paragraphs.

The operational procedure is the same as the procedure for the operation and analysis described in (1) above, however, determining parameters were set. The initial temperature was 25° C. The temperature was increased to 800° C. at a rate of 20° C. per minute, and then maintained at 800° C. for 15 minutes. The weight of the sample was controlled at 3-10 mg.

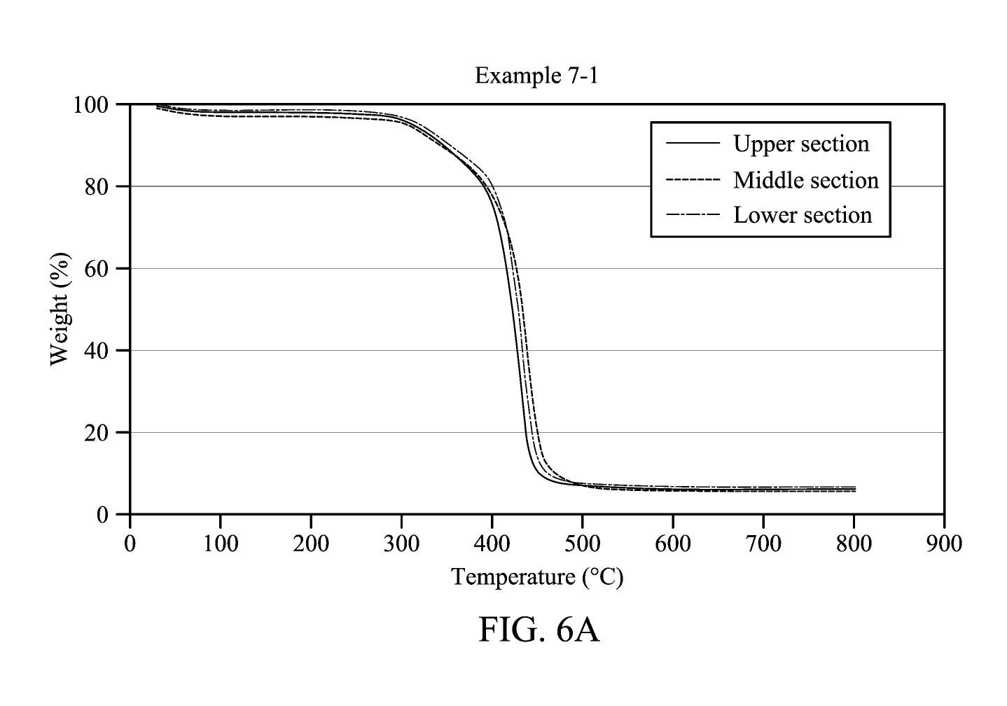

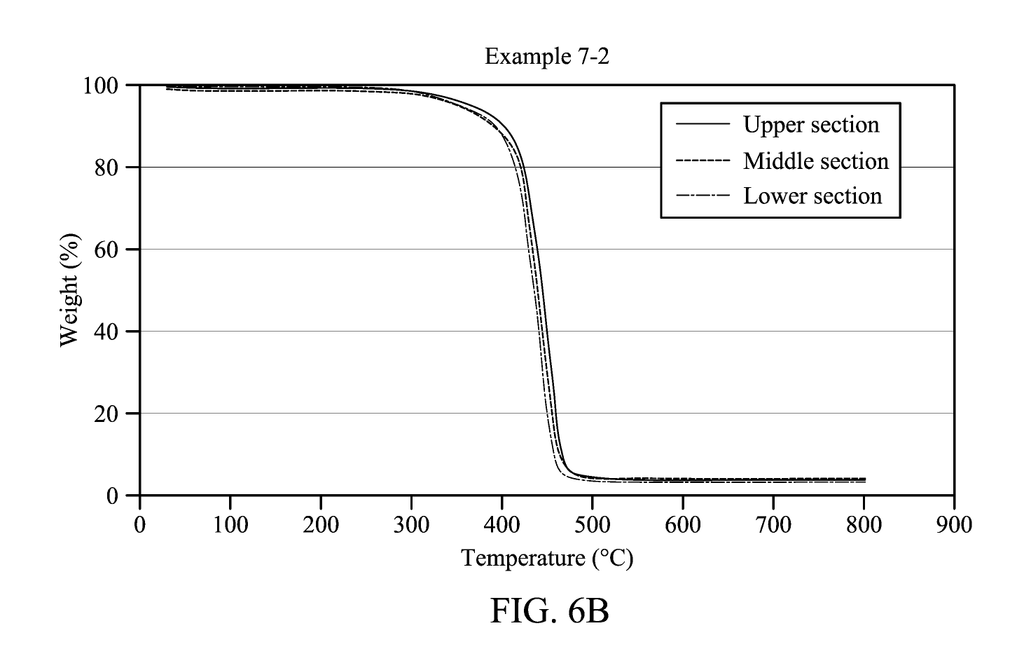

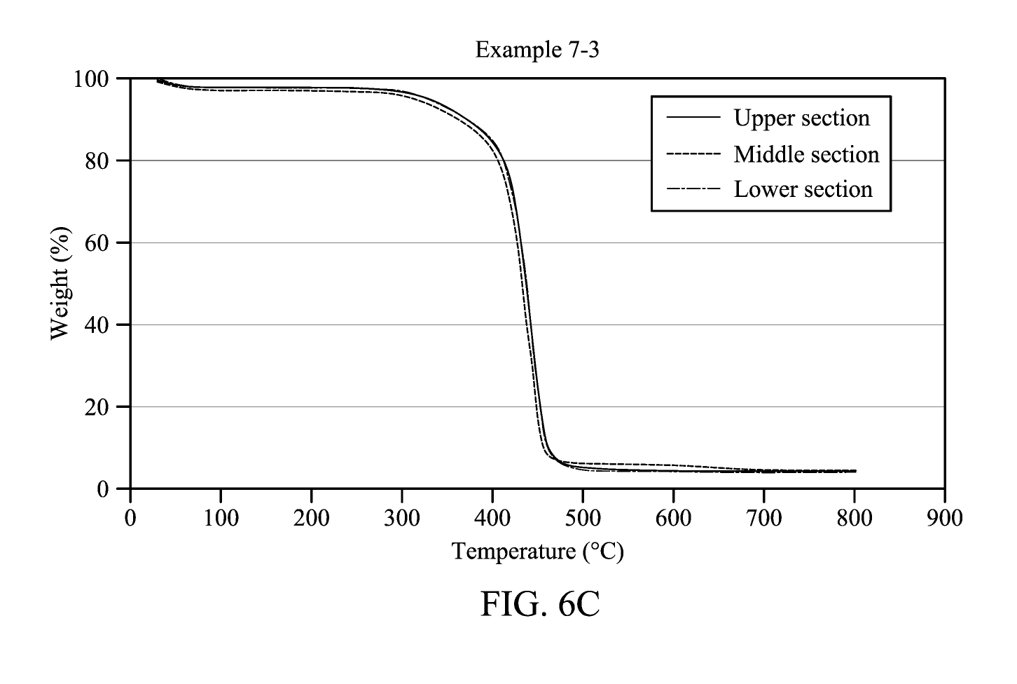

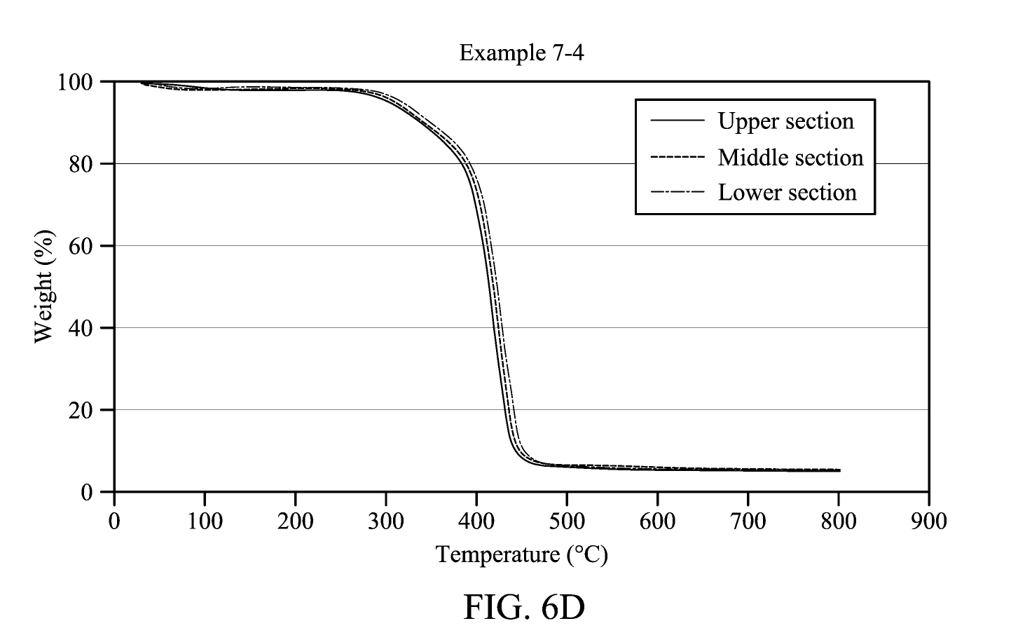

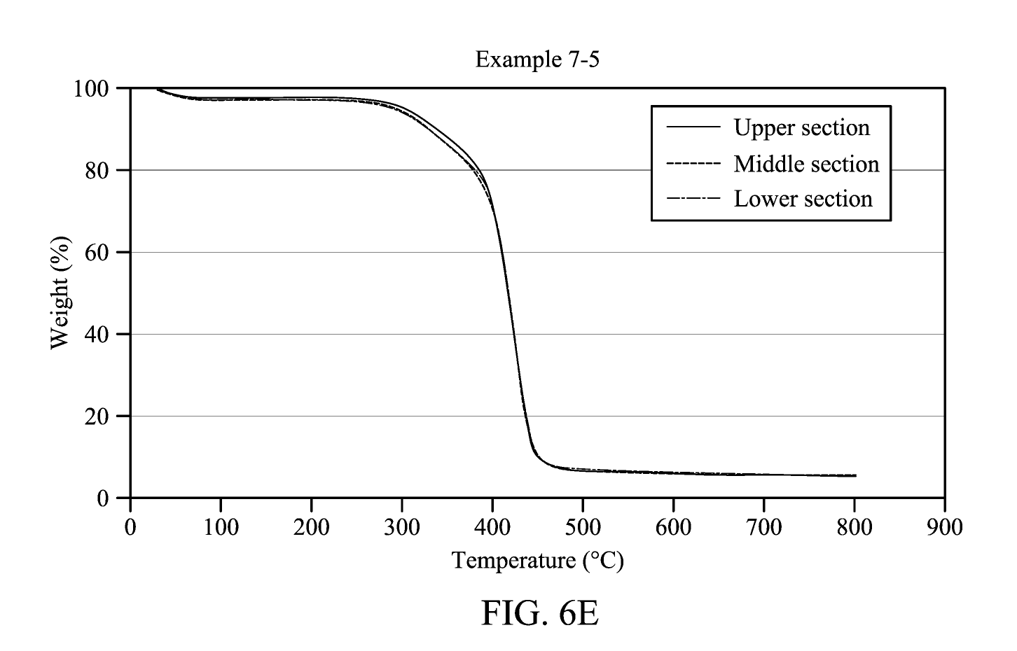

Double layer films prepared by Examples 7-1 to 7-5 were observed for composition uniformity at different locations (a film was cross cut into three sections: an upper section, a middle section, and a lower section) using a thermogravimetric analyzer. The results are shown in FIGS. 6A to 6E.

FIGS. 6A, 6B, 6C, 6D and 6E show thermogravimetric analysis results for the double layer films prepared by Example 7-1, Example 7-2, Example 7-3, Example 7-4 and Example 7-5, respectively. FIGS. 6A to 6E show that all film almost shows only one curve. Accordingly, it is known that the double layer films formed by the method of the present disclosure, even if in a condition of different proportion of ingredients and different ingredients, still can uniformly form films.

2. Fourier Transform Infrared Spectrometry (FT-IR) Analysis

The principle behind Fourier transform infrared spectrometry is that, for a molecule, when vibration-rotation occurs at various bonding structures in the molecule, the molecule absorbs appropriate infrared energy to obtain a spectrometry. Since infrared spectrometry can provide information about the properties of a molecular structure, and except for optical isomers, there is almost no identical spectrometry for different organic compounds, the structure and the properties of oscillating bonds or rotating bonds can be discerned by investigations of infrared spectrometry, and the presence and content of a compound can be identified or analyzed at the same time.

(1) Fourier Transform Infrared Spectrometry (FT-IR) Analysis for the Film Prepared by Example 3-1

The operation procedure for Fourier transform infrared spectrometry analysis is described in the following paragraphs.

The film prepared in Example 3-1 (polycaprolactone/poly(lactic-co-glycolic acid)/gelatin) was cut to fit a size required by the stage of the equipment to be ready-for-use.

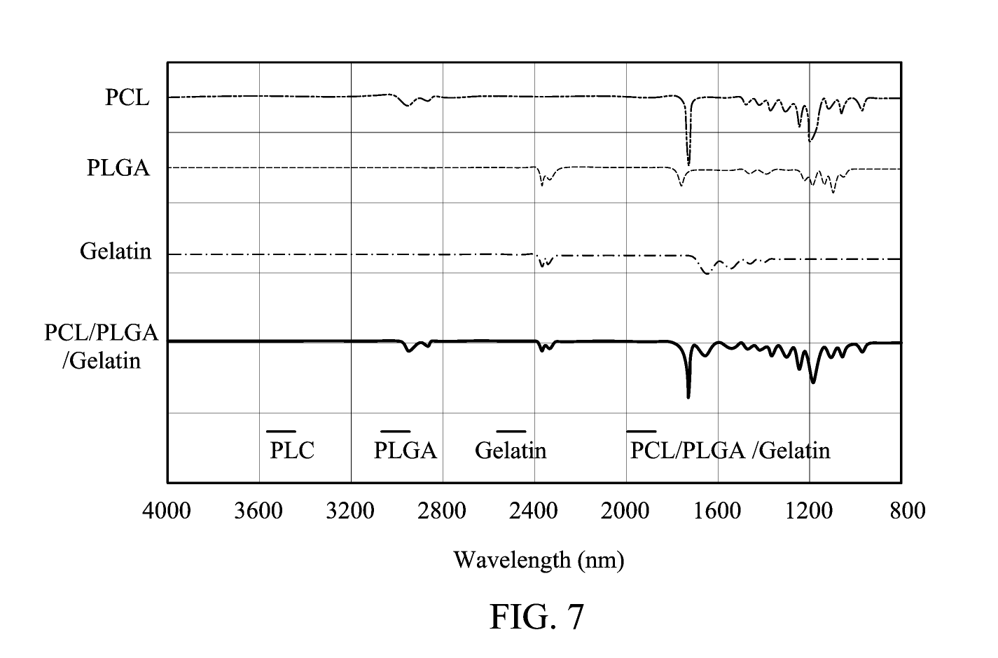

First, a sample stage cleaning was performed by wiping the sample stage with ethanol and leaving it to stand for 1 minute to let the ethanol volatilize. After the ethanol volatilized, the sample stage was pressed down to be fixed (without placing any matter thereon). The “Spectrometer setup” button of the software was clicked to enter a settings screen to determine that the laser intensity of the equipment was stable. After the determination, the “Background” button was clicked to detect the background level. After the detection, a file name was set and the whole working file was saved in a folder. The sample to be tested was placed on the sample stage, and if the sample was a film sample, the surface to be tested was face-down, and the stage was pressed down to be fixed to perform a scan. The “Scan” button in the “Scan” item in the toolbar was clicked to enter parameter settings, and “Scans” was set to 32, “Resolution” was set to 4, and “Truncation Range” was set to manual mode, and the range was set to 4000-800. Since the detecting chip of the sample stage had an absorbing effect on light with a wavelength less than 700, after removing the noise region, the range of 4000-800 was selected and the background level previously detected was deducted, and then the completed spectrometry file was saved and analyzed. The result is shown in FIG. 7.

According to FIG. 7, the characteristic peaks for polycaprolactone included 2945 cm⁻¹ (CH₂ characteristic peak), 1724 cm⁻¹ (C═O [[s]] characteristic peak), 2864 cm⁻¹ (CH₂ stretching) and 1242 cm⁻¹ (COC characteristic peak), characteristic peaks for poly(lactic-co-glycolic acid) include 1754 cm⁻¹ (C═O characteristic peak), 1184 cm⁻¹ (COC characteristic peak) and 1192 cm⁻¹ (CO characteristic peak). Characteristic peaks for gelatin included 1629 cm⁻¹ (—C(O)NH₂ characteristic peak, amide I) and 1523 cm⁻¹ (—C(O)NH₂ characteristic peak, amide II), and the peak for the amide group located at 1652 cm⁻¹. This peak indicted that gelatin randomly curled, and indicted bonds for α-helix structure.

Moreover, FIG. 7 showed that the characteristic peaks for polycaprolactone, poly(lactic-co-glycolic acid) and gelatin all appeared in the Fourier transform infrared spectrometry for the film prepared in Example 3 (polycaprolactone/poly(lactic-co-glycolic acid)/gelatin), and this indicated that polycaprolactone, poly(lactic-co-glycolic acid) and gelatin of the film prepared in Example 3 were blended without chemical crosslinking, and polycaprolactone, poly(lactic-co-glycolic acid) and gelatin were uniformly blended since the characteristic peaks for these three substances appeared in the same section of the film.

(2) Fourier Transform Infrared Spectrometry (FT-IR) Analysis for the Double Films Prepared by Examples 7-1 to 7-5

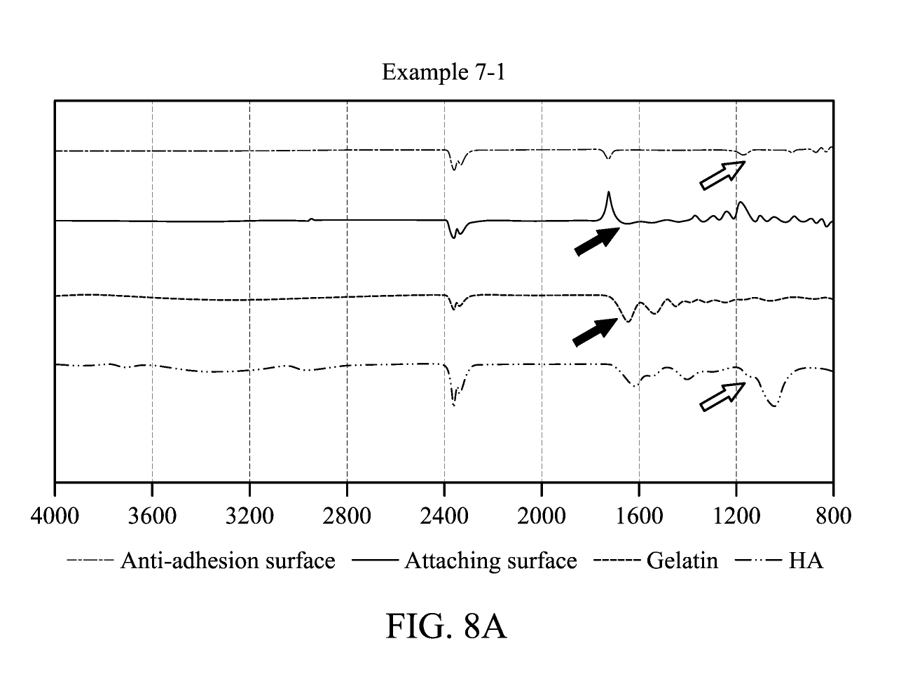

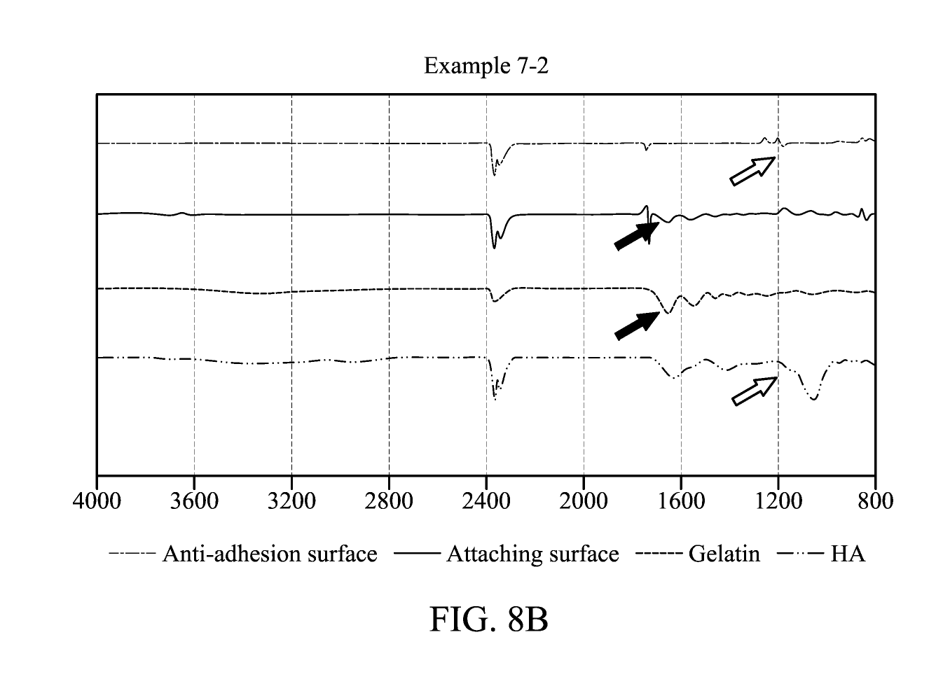

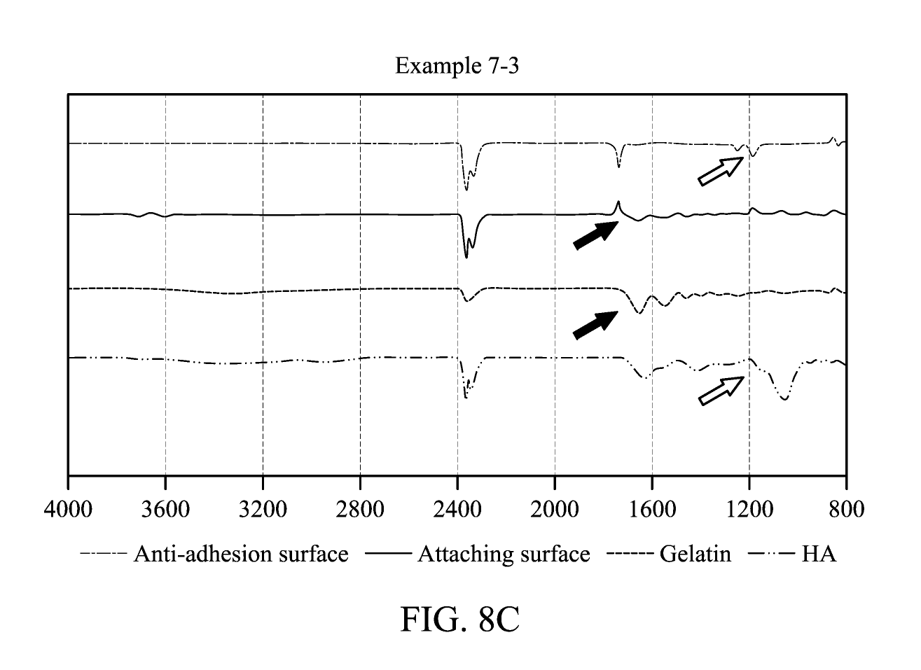

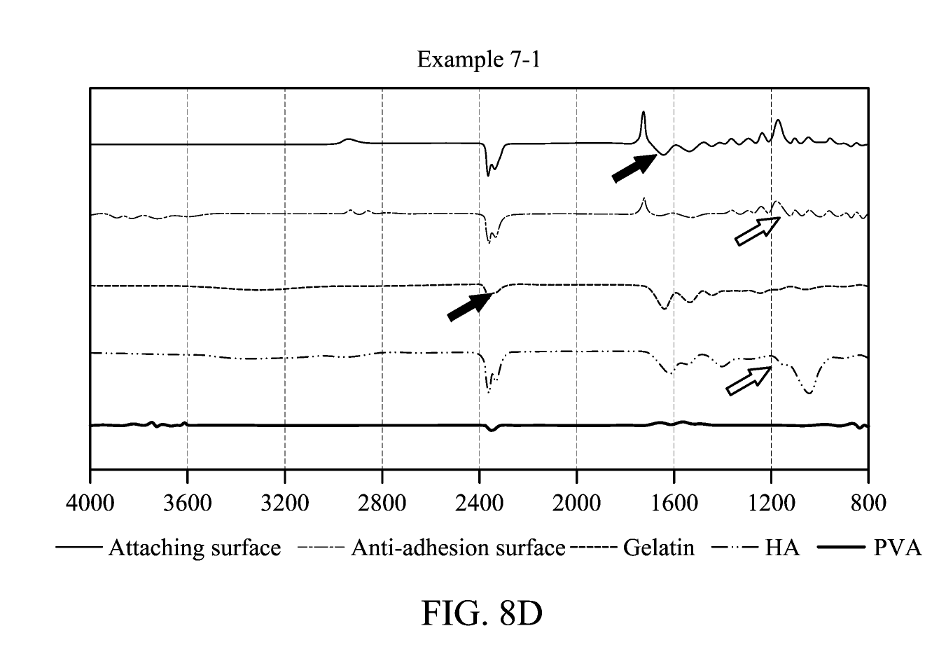

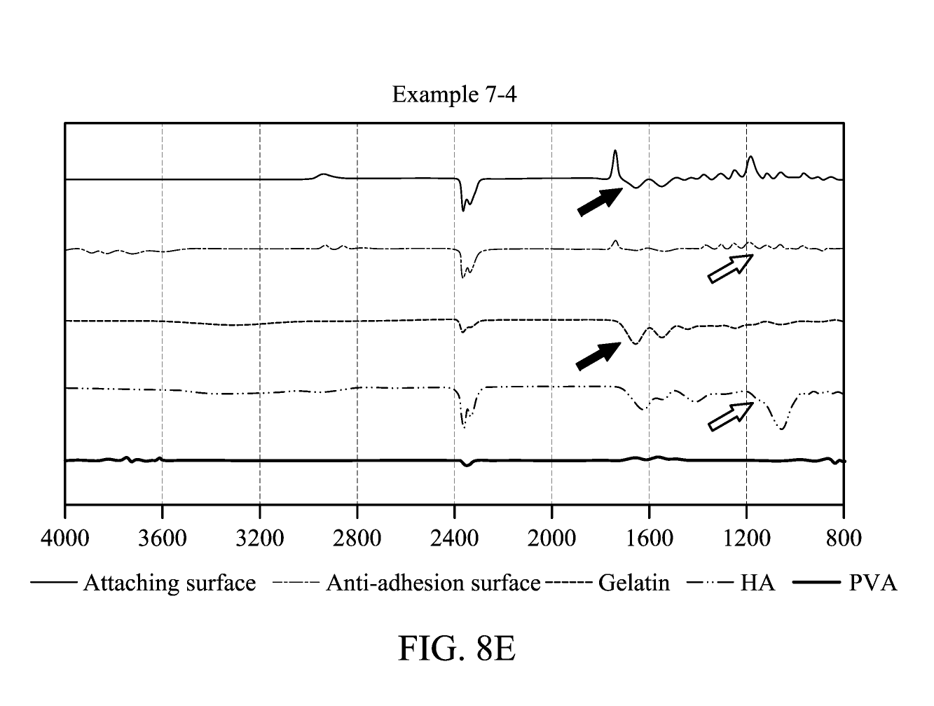

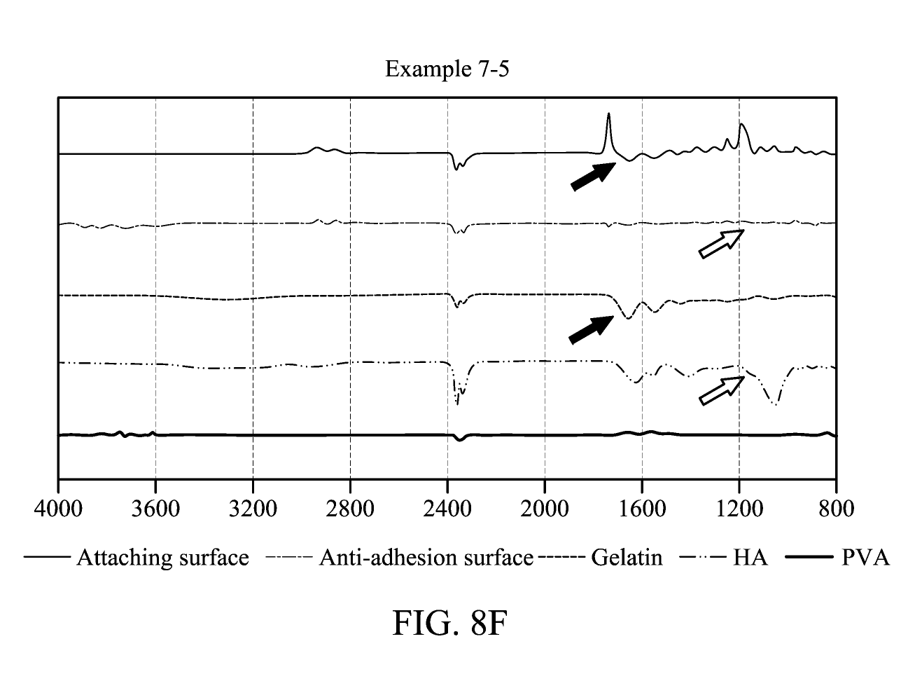

Fourier transform infrared spectrometry (FT-IR) analysis was performed on the double films prepared by Examples 7-1 to 7-3 and the double films prepared by Example 7-1, Example 7-4 and Example 7-4 in two batches. The procedure for operation and analysis described in (1) above can be referred to, however, the determining parameters and conditions were set: the analysis range was 4000-400 cm⁻¹, and spectral conditions for analyzing the resolution was 16 cm⁻¹, 8 cm⁻¹ or 4 cm⁻¹, depending on analysis conditions. The results are shown in FIGS. 8A to 8F.

FIGS. 8A, 8B and 8C show Fourier transform infrared spectrometry (FT-IR) analysis results for the double layer films prepared by Example 7-1, Example 7-2 and Example 7-3. FIGS. 8D, 8E and 8F show Fourier transform infrared spectrometry (FT-IR) analysis results for the double layer films prepared by Example 7-1, Example 7-4 and Example 7-5. FIGS. 8A to 8F show that all attachment layers of the all double layer film have a peak the same as gelatin standard of 1800-1600 cm⁻¹ (indicated by the black arrow) while all anti-adhesion layers of the all double layer film have a peak the same as hyaluronic acid standard of 1200-1000 cm⁻¹ (indicated by the hollow arrow). Accordingly, it is known that for the double layer films formed by the method of the present disclosure, the two layers indeed have different components.

3. Analysis of Theoretical Proportion and Actual Proportion of Ingredients of the Films

Analysis for theoretical proportion of ingredients or analysis for theoretical proportion and actual proportion of ingredients was performed on the double layer films prepared by Examples 7-1 to 7-5

(1) Analysis of Theoretical Proportion

Analysis for theoretical proportion of ingredients was performed on the double layer films prepared by Examples 7-1 to 7-5.

Theoretical proportion of each ingredient was calculated according to weight of each ingredient used to prepare the films. The results are shown in Table 1.

(2) Analysis for Actual Proportion of Ingredients was Performed on the Double Layer Films Prepared by Examples 7-1 to 7-3.

The operation procedure is described in the following paragraphs.

A glass vial for dissolving, filter paper, and a glass vial for filtering were provided and weighed to obtain the first weight of each (the empty weight).

The film was sampled 1±0.005 g and placed in the aforementioned glass vial for dissolving. After that, an appropriate amount (10 ml) of DCM was added to the aforementioned glass vial for dissolving and left to stand overnight to dissolve the film sample.

After the aforementioned step, the solution of the glass vial for dissolving was added to a glass syringe and filtered by the filter mentioned above and the filtrate was collected in the aforementioned glass vial for filtering. After the appropriate amount (10 ml) of DCM was used to wash the glass vial for dissolving, and the solution of the glass vial for dissolving was filtered by the aforementioned filter again and the filtrate was also collected in the aforementioned glass vial for filtering.

Then, the glass vial for dissolving, the filter paper, and the glass vial for filtering were placed in a fume hood until the solvent completely volatilized.

After that, the glass vial for dissolving, the filter paper, and the glass vial for filtering were weighed again to obtain the weights of the glass vial for dissolving, the filter paper, and the glass vial for filtering after the aforementioned treatments.

The sum of the first weights of the glass vial for dissolving and the filter was subtracted from the sum of the second weight of the glass vial for dissolving and the filter to obtain the weight of the hydrophilic ingredients in the film.

The first weights of the glass vial for filtering was subtracted from the second weight of the glass vial for filtering to obtain the weight of the polycaprolactone in the film.

The actual proportion of each ingredient was calculated according to the weight of the hydrophilic ingredients in the film and the weight of the polycaprolactone in the film, and the results are shown in Table 2.

TABLE 1
Theoretical proportion of ingredients of the films

| Formulation | Polycaprolactone | | Hyaluronic | Polyvinyl | Total | Gelatin + |
|---|---|---|---|---|---|---|
| for double | (wt %) | Gelatin | acid | alcohol | percentage | Hyaluronic |
| layer film | Hydrophobic | (wt %) | (wt %) | (wt %) | (wt %) | acid (wt %) |
| Example 7-1 | 67.797 | 31.356 | 0.847 | 0.000 | 100.000 | 32.203 |
| Example 7-2 | 72.464 | 26.812 | 0.725 | 0.000 | 100.000 | 27.537 |
| Example 7-3 | 75.949 | 23.418 | 0.633 | 0.000 | 100.000 | 24.051 |
| Example 7-4 | 67.227 | 31.092 | 0.840 | 0.840 | 100.000 | 31.932 |
| Example 7-5 | 66.667 | 30.833 | 0.833 | 1.667 | 100.000 | 31.666 |

TABLE 2
Actual proportion of ingredients of the films

| Formulation for | Polycaprolactone | Gelatin + Hyaluronic acid |
|---|---|---|
| double layer film | (wt %) | (wt %) |
| Example 7-1 | 62 (−6) | 38 (+6) |
| Example 7-2 | 77 (+5) | 23 (−5) |
| Example 7-3 | 78 (+2) | 22 (−2) |

( ): Value in the parentheses represents the difference as compared to the theoretical value.

According to Table 1 and Table 2, it is known that in the double layer film of the present disclosure, the range of the proportion of the hydrophobic material, polycaprolactone, is about 55-80 wt % while the range of the proportion of the hydrophilic material is about 20-30 wt %.

3. Physicochemical Properties for Films

(1) Standard Test of Burst Strength

(1) Test of Burst Strength for Films Prepared in Comparative Example 1, Example 2-1 and Example 3-1

Tests of burst strength were performed on the films prepared in Comparative Example 1, Example 2 and Example 3-1, and the commercial sealing film (TachoSil) and sealing patch (TissuePatch) (formed by polylactic acid (PLA), two layer structure, attachment effect is achieved by chemical covalent bonds) according to Standard Test Method for Burst Strength of Surgical Sealants) defined by ASTM F2392.

Operation procedure for test of burst strength according to ASTM F2392 is summarized in the following.

The film to be tested was cut to a round piece with a diameter of 1.5 cm, and then the round piece was attached to pig intestines and kept at 37° C. for 15 minutes to prepare a pig intestine testing sample. Next, the prepared pig intestine testing sample was set on a testing mold for water pressure, and the burst test was performed on the testing mold for water pressure by a peristaltic pump with a flow rate of 3 ml/minute.

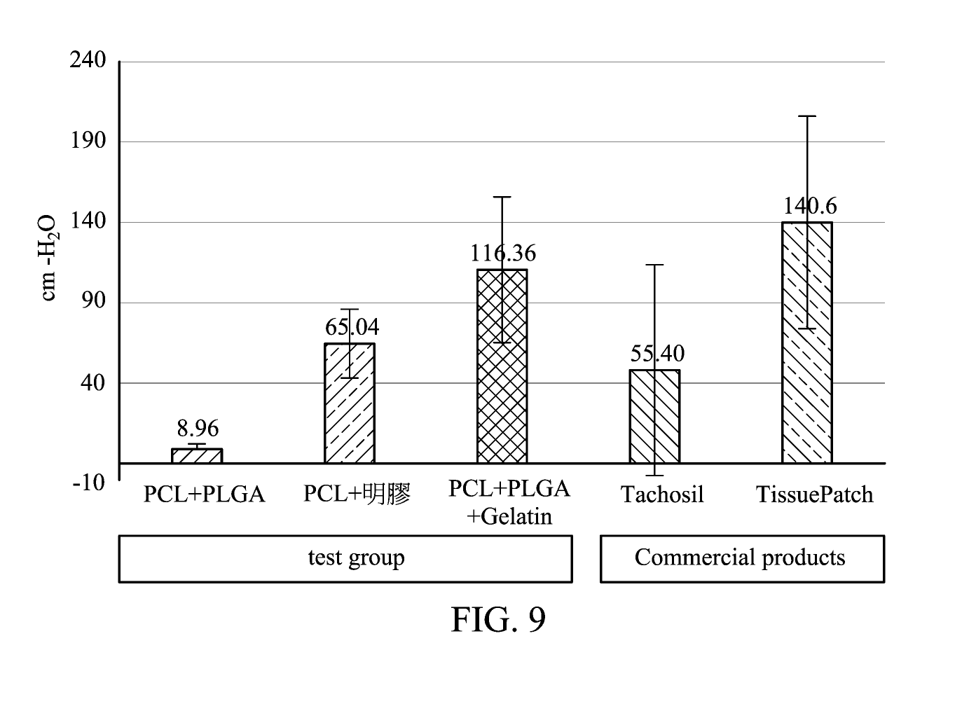

The results are shown in FIG. 9.

FIG. 9 shows that the burst strength of the film prepared in Example 3-1 (polycaprolactone/poly(lactic-co-glycolic acid)/gelatin) is higher than that of the sealing film (TachoSil) in clinical use at present, and it matches that of the sealing patch (TissuePatch) presently in clinical use.

(1-2) Tests of Burst Strength for the Films Prepared in Examples 7-1 to 7-5 (Double Layer Film)

Similarly, tests of burst strength were performed on the films prepared in Examples 7-1 to 7-5 and sealing patch in clinical use (TissuePatch) (formed by polylactic acid (PLA), two layer structure, attachment effect is achieved by chemical covalent bonds) based on Standard Test Method for Burst Strength of Surgical Sealants) defined by ASTM F2392.

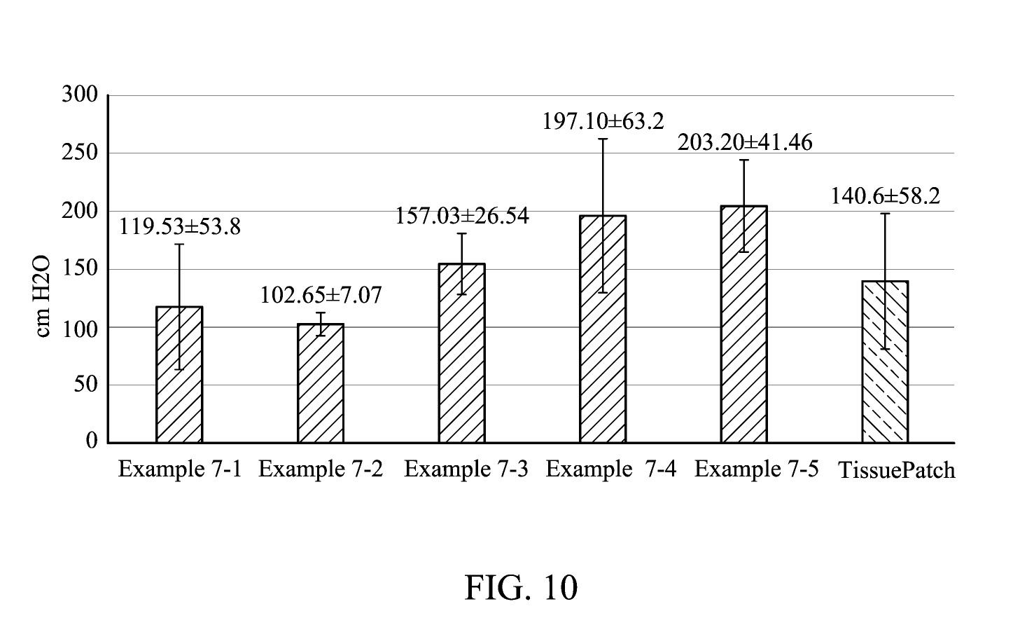

The operation procedure for the test can be found in the description of the operation procedure described in (1-1) above. The test results are shown in FIG. 10.

FIG. 10 shows that the strength of the double layer films prepared in Examples 7-1 to 7-5 is higher than that of the sealing patch formed by polylactic acid (TissuePatch) presently in clinical use.

(2) Test of Tensile Properties

(2-1) Tensile Tests for Films Prepared in Comparative Example 1, Example 2-1 and Example 3-1

Tensile tests were performed on the films prepared in Comparative Example 1, Example 2-1 and Example 3-1, and the commercial sealing film (TachoSil) used clinically at present and the sealing patch formed by polylactic acid (TissuePatch) according to Standard Test Method for Tensile Properties of Thin Plastic Sheeting defined by ASTM D882-12.

Operation procedure for tensile tests according to ASTM D882-12 is summarized in the following.

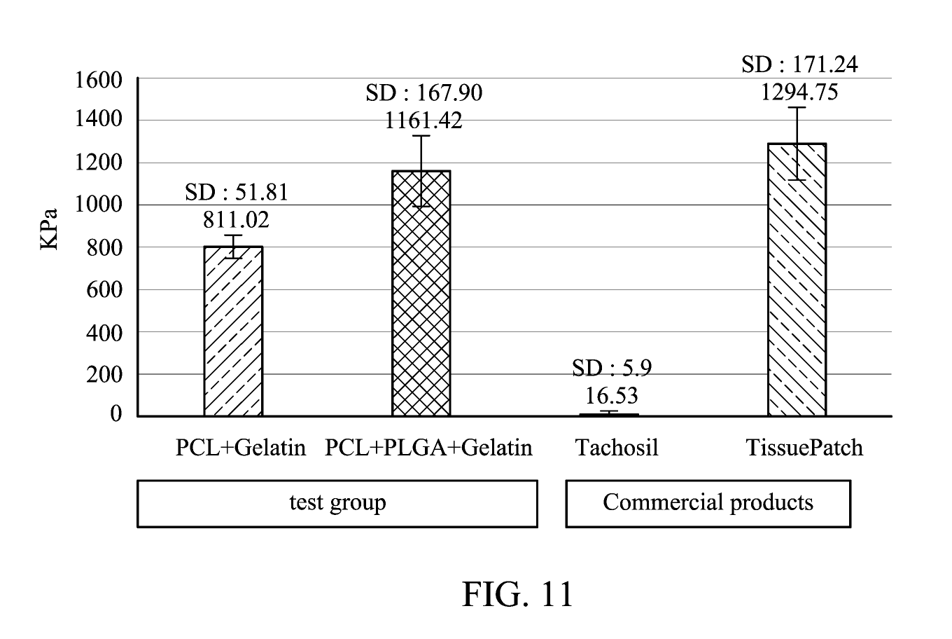

ASTM D882-12 standard is used to determine tensile properties, especially suitable for a plastic film with a thickness of less than 1 mm. Base on this standard, a test specimen was cut using a sharp cutter to a strip of 100*25.4 mm², and the initial distance between the upper and lower pneumatic chucks were adjusted to 100 mm, and the pulling speed was set to 50 mm/minute. The test results are shown in FIG. 11.

FIG. 11 shows that since the film prepared in Example 3-1 (polycaprolactone/poly(lactic-co-glycolic acid)/gelatin) combines the soft and perfect fitting properties of polycaprolactone with the mechanical strength of poly(lactic-co-glycolic acid), the tensile strength thereof is much higher than that of the sealing film (TachoSil) used clinically at present, and matches that of the sealing patch (TissuePatch) used clinically at present.

(2-2) Tensile Tests for the Films Prepared in Examples 7-1 to 7-5 (Double Layer Film)

Similarly, tensile tests were performed on the films prepared in Examples 7-1 to 7-5 and sealing patch in clinical use (TissuePatch) based on Standard Test Method for Tensile Properties of Thin Plastic Sheeting defined by ASTM D882-12.

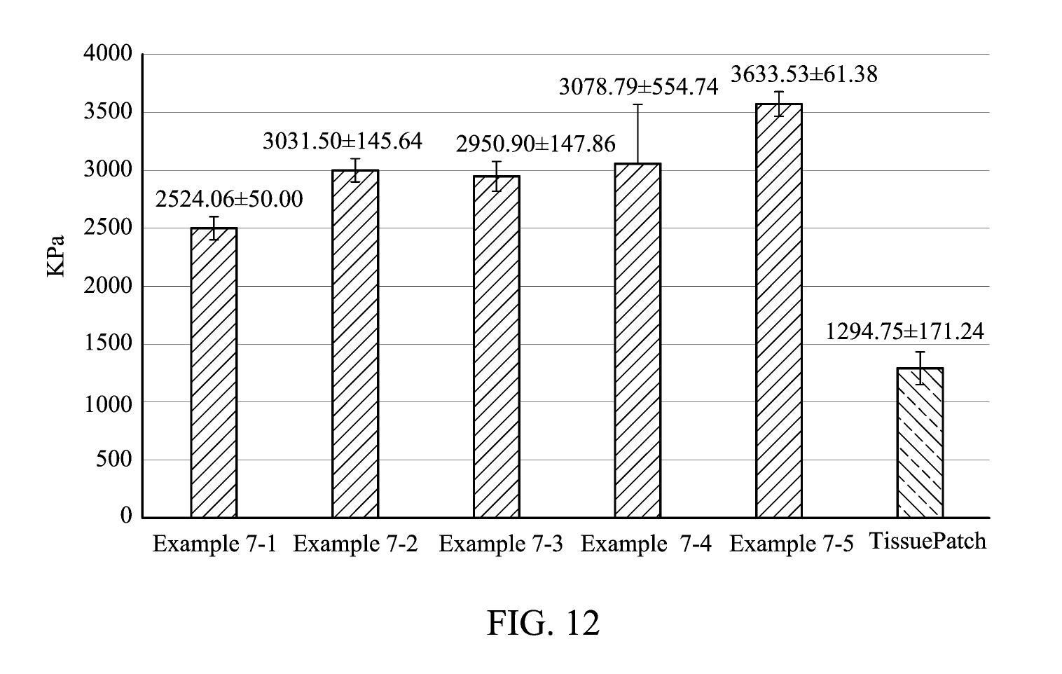

The operation procedure for the test is the same as the operation procedure described in (2-1) above, although the tensile rate was set 12.5 mm/minute. The test results are shown in FIG. 12.

FIG. 12 shows that, compared to the sealing patch presently in clinical use (TissuePatch), the double layer films prepared in Examples 7-1 to 7-5 have a tensile strength that is much higher. This shows that double layer films prepared in Examples of the preset disclosure have extremely excellent mechanical strength.

(3) Test of Suture Pullout Strength

Tensile of suture were performed on the films prepared in Examples 7-1 to 7-5 and sealing patch in clinical use (TissuePatch) based on the testing method for suture tensile for films recited in the literature (Physicomechanical evaluation of absorbable and nonabsorbable barrier composite meshes for laparoscopic ventral hernia repair, Surgical EndoscopySurg Endosc. 2011 May; 25(5):1541-52). Two batches of experiments were performed, wherein the films prepared in Examples 7-1 to 7-3 and sealing patch in clinical use (TissuePatch) were tested in one batch of experiments while the films prepared in Example 7-1, Example 7-4 and Example 7-5 and sealing patch in clinical use (TissuePatch) were tested in another batch of experiments.

Operation procedure for the testing method for suture tensile for films recited in the literature (Physicomechanical evaluation of absorbable and nonabsorbable barrier composite meshes for laparoscopic ventral hernia repair, Surgical EndoscopySurg Endosc. 2011 May; 25(5):1541-52) is summarized in the following.

Testing Parameters for Suture Pullout:

PE suture size: 1-0 size;

Determining instrument: Tension testing machine (Instron 4467);

Range of the testing force: 0-5000 N.

The film to be tested was cut to a test specimen with a size of 2.5×7.6 cm². Next, after the PE suture passed through a central point of a position which was at a distance of 1 cm from the bottom of the film, the PE suture was pulled down at a tensile rate of 300 nm/minute (12 in/minute) to test the tensile strength.

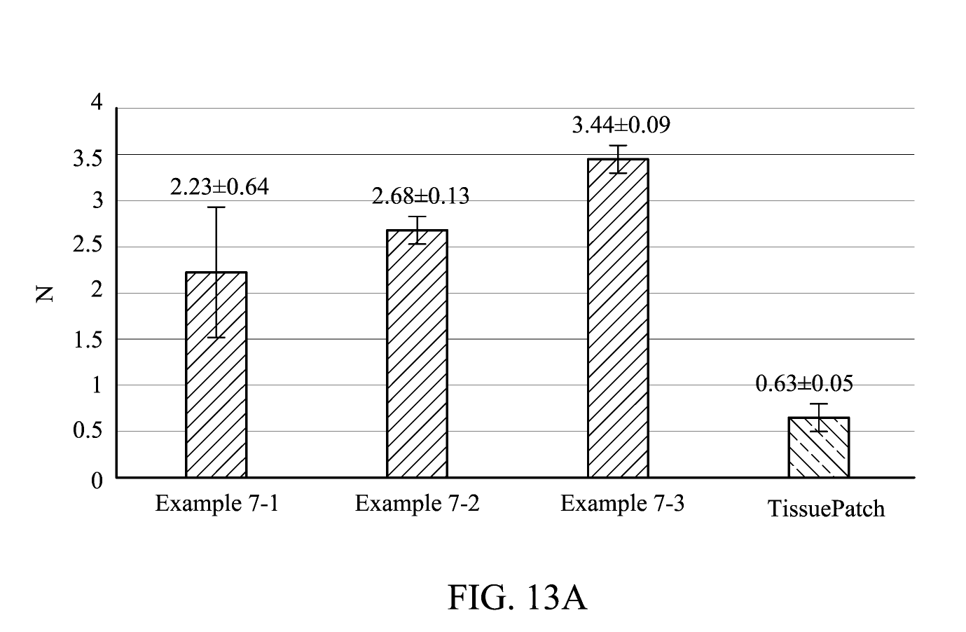

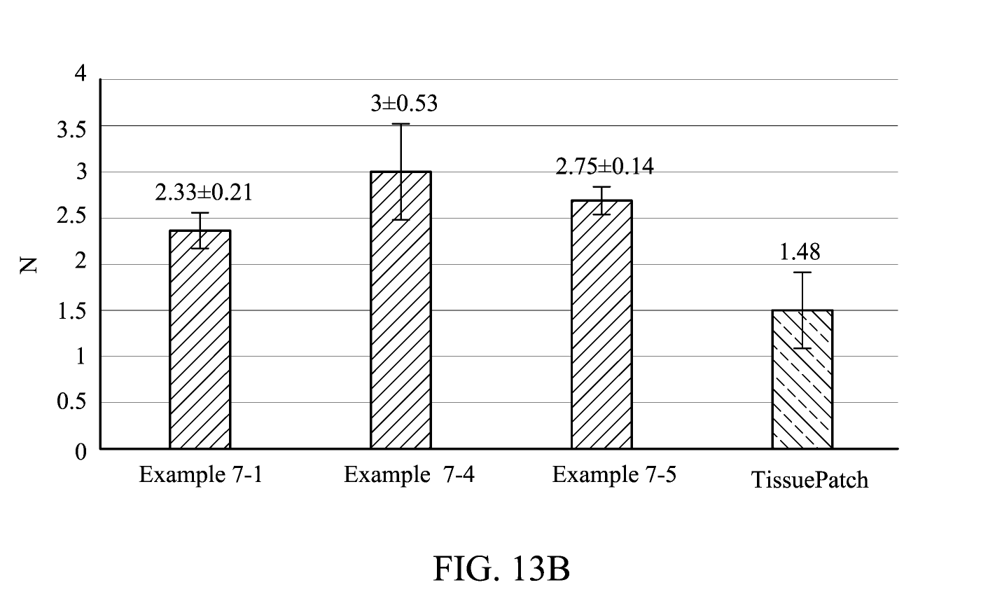

The results are shown in FIG. 13A and FIG. 13B.

According to FIG. 13A and FIG. 13B, it is known that as compared to the sealing patch presently in clinical use (TissuePatch), the tensile strength of the double layer films prepared in Examples 7-1 to 7-5 is much higher. This shows that double layer films prepared in Examples of the preset disclosure have extremely excellent mechanical strength.

(4) Tests of Tear Resistance

Tests of Tear Resistance of the Films Prepared in Examples 7-1 to 7-5

Tests of tear resistance were performed on the films prepared in Examples 7-1 to 7-5 and sealing patch in clinical use (TissuePatch) based on Standard Test Method for Tensile Properties of Thin Plastic Sheeting defined by ASTM D1004. Two batches of experiments were performed, wherein the films prepared in Examples 7-1 to 7-3 and sealing patch in clinical use (TissuePatch) were tested in one batch of experiments while the films prepared in Example 7-1, Example 7-4 and Example 7-5 and sealing patch in clinical use (TissuePatch) were tested in another batch of experiments.

Operation procedure for tensile tests according to ASTM D1004 is summarized in the following.

Tensile tests were performed according to ASTM D1004. The test specimen was designed based on the standard content, the test of tear resistance was performed by an universal tensile testing machine by a tensile rate of 51±5%/minute.

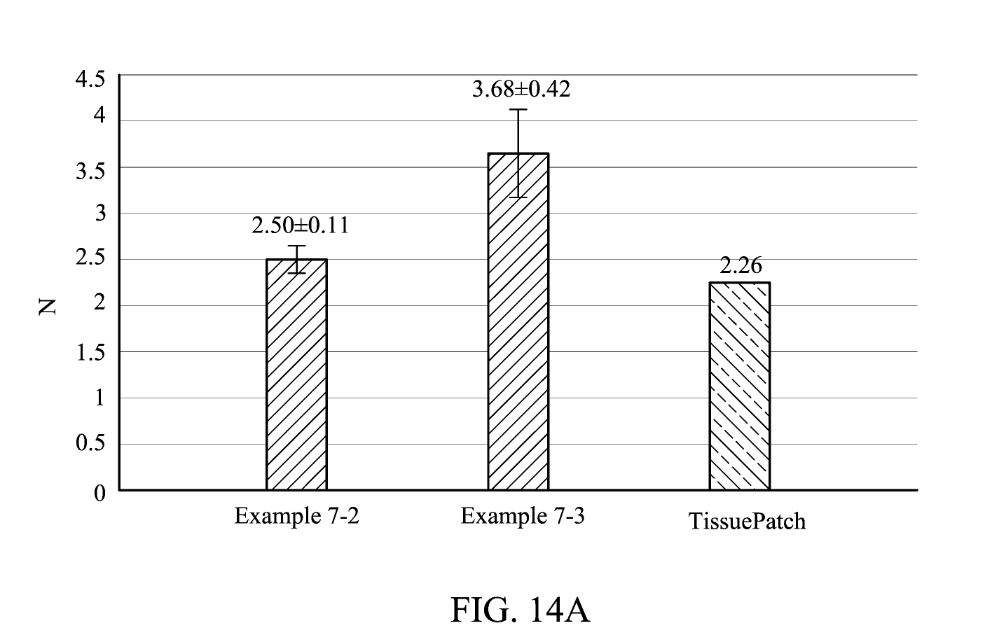

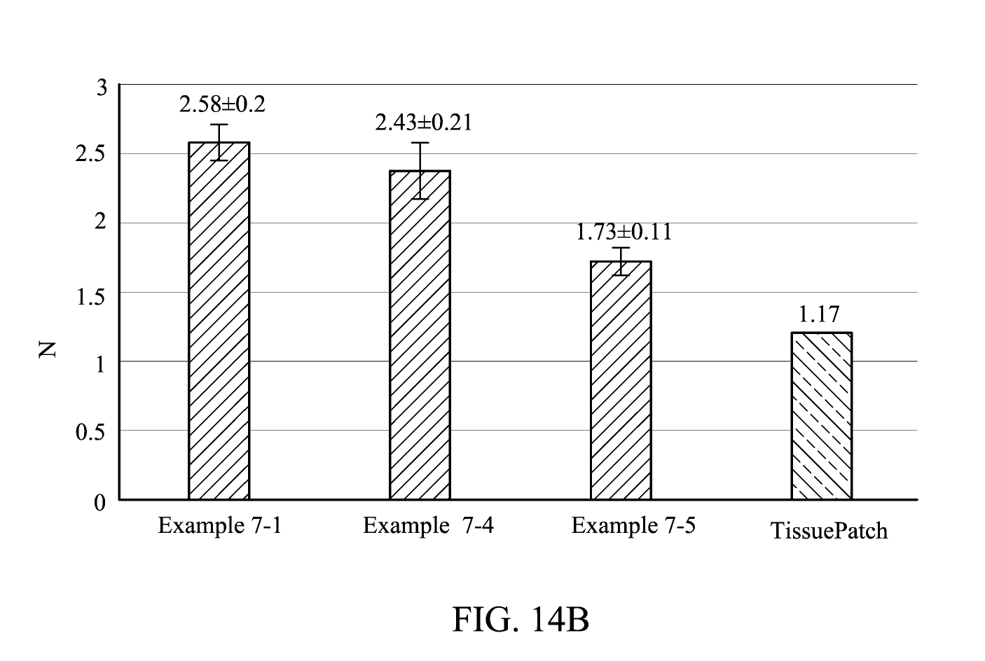

The results are shown in FIGS. 14A and 14B.

According to FIG. 14A and FIG. 14B, it is known that as compared to the sealing patch presently in clinical use (TissuePatch), the tear resistance of the double layer films prepared in Examples 7-1 to 7-5 is much higher. This shows that double layer films prepared in Examples of the preset disclosure have extremely excellent mechanical strength.

3. Surface Structure and Roughness of Films

(1) Observation of Surface Structure

(1-1) Observation of Surface Structure of the Films Prepared in Example 3-1 and Comparative Example 1

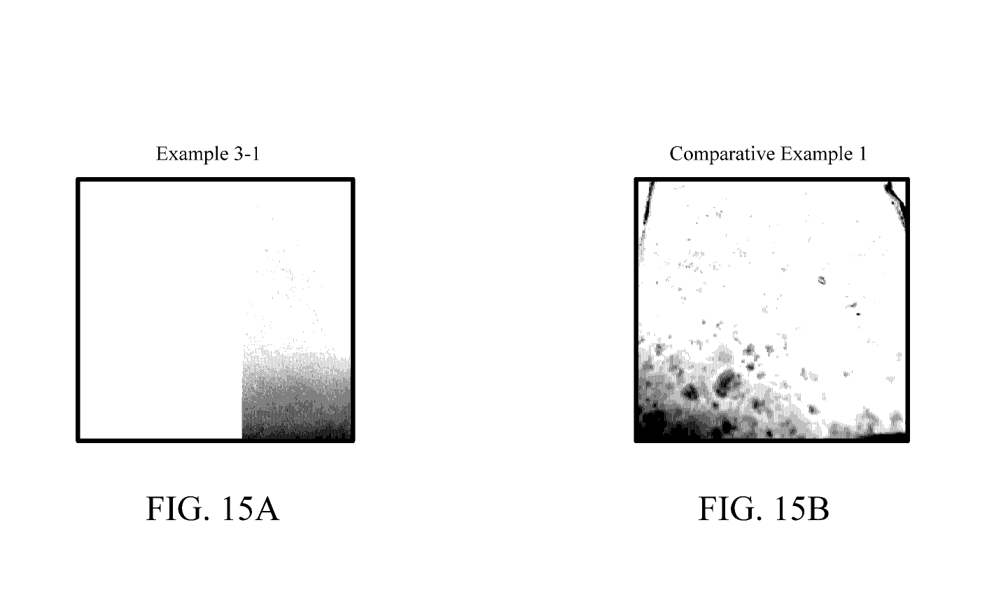

A film prepared using the method of the present disclosure (the film prepared in Example 3-1 (polycaprolactone/poly(lactic-co-glycolic acid)/gelatin)) and a film prepared without using the process of the present disclosure (the film prepared in Comparative Example 1 (polycaprolactone/poly(lactic-co-glycolic acid))) were observed with a microscope to perform a surface structure observation of the two films. The results are shown in FIGS. 15A and 15B.

The results show that the surface of the film prepared using the process of the present disclosure (the film prepared in Example 3-1 (polycaprolactone/poly(lactic-co-glycolic acid)/gelatin)) is uniform without phase separation or particles (FIG. 15A).

In contrast, there was serious phase separation on the surface of the film prepared without using the process of the present disclosure (the film prepared in Comparative Example 1 (polycaprolactone/poly(lactic-co-glycolic acid))), and the film could not even take shape (FIG. 15B).

(1-2) Observation of Surface Structure of the Films Prepared in Example 2-2, Example 3-2 and Example 7-1

The films prepared in Example 2-2, Example 3-2 and Example 7-1 were observed by SEM.

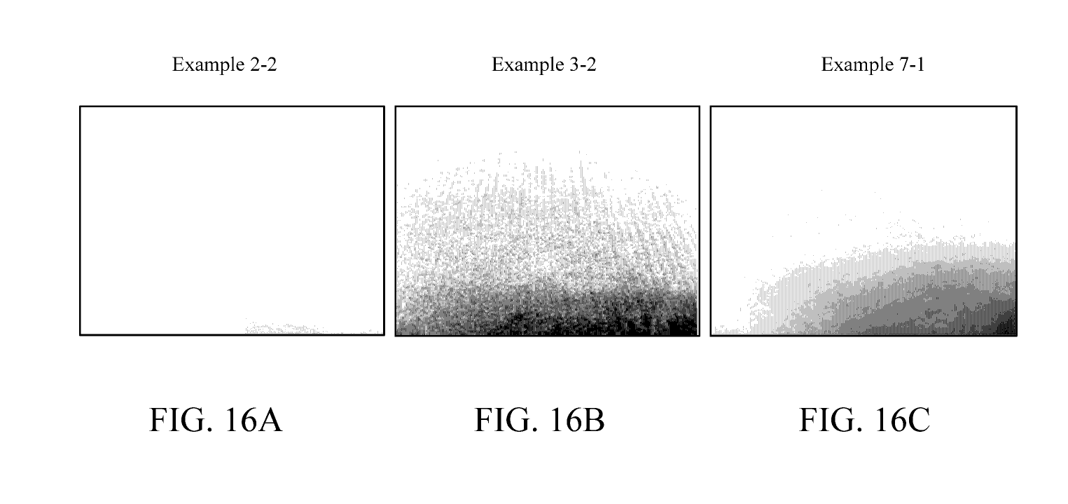

Double sided adhesive carbon tape specific to SEM was attached to the carrier to be operated. Next, after the film was cut into a sample with a size of 0.5×0.5 cm², the cut sample was attached to the double sided adhesive carbon tape and gold-plating was performed on the surface of the sample, and time for the gold-plating was 90 seconds. After the gold-plating was completed, the film sample was loaded on the SEM machine and observed by using a voltage of 3-5 kV and a magnification of 35X. The results are shown in FIGS. 16A, 16B and 16C.

FIGS. 16A, 16B and 16C show the appearances of the films prepared in Example 2-2, Example 3-2 and Example 7-1, respectively.

FIGS. 16A, 16B and 16C show that the appearances of the films prepared in Example 2-2, Example 3-2 and Example 7-1 all are uniform and smooth surface structures.

(2) Roughness

(2-1) Roughness Analysis for the Films Prepared in Example 2-1, Example 3-1 and Comparative Example 1

Surface roughness of films prepared by the method of the present disclosure (the films prepared in Example 2-1 and Example 3-1) and a film prepared without using the process of the present disclosure (the film prepared in Comparative Example 1) were determined according to Standard Test Method for Surface Roughness defined by ASTM D7127-13.

Measurement was performed according to ASTM D7127-13 standard. The measuring instrument used was Surfcorder SE1700. The principle behind this measurement is that, by scanning the height difference of a test specimen with a probe through a light reflection and using scattering transmission, the reflective signals from the light sources which are on and under the probe were converted and calculated to draw a sectional drawing to determine the film thickness variation and surface roughness. After that, full roughness (Rz) was calculated.

Calculation for full roughness (Rz) is summarized in the following paragraph.

An estimated length was divided equally into 5 aliquots of a sample length, and the distance between the highest point and the lowest point in each aliquot of the sample length was calculated, and then the all the distances between the highest point and the lowest point in each aliquot of the sample length are summarized and averaged to obtain full roughness.

Formula for full roughness is shown in the following:

$$Rz = \frac{{R}_{y 1} + {R}_{y 2} + {R}_{y 3} + {R}_{y 4} + {R}_{y 5}}{5} ,$$

wherein R_{y1}, R_{y2}, R_{y3}, R_{y4} and R_{y5} respectively represent the distance between the highest point and the lowest point in the first aliquot to the fifth aliquot.

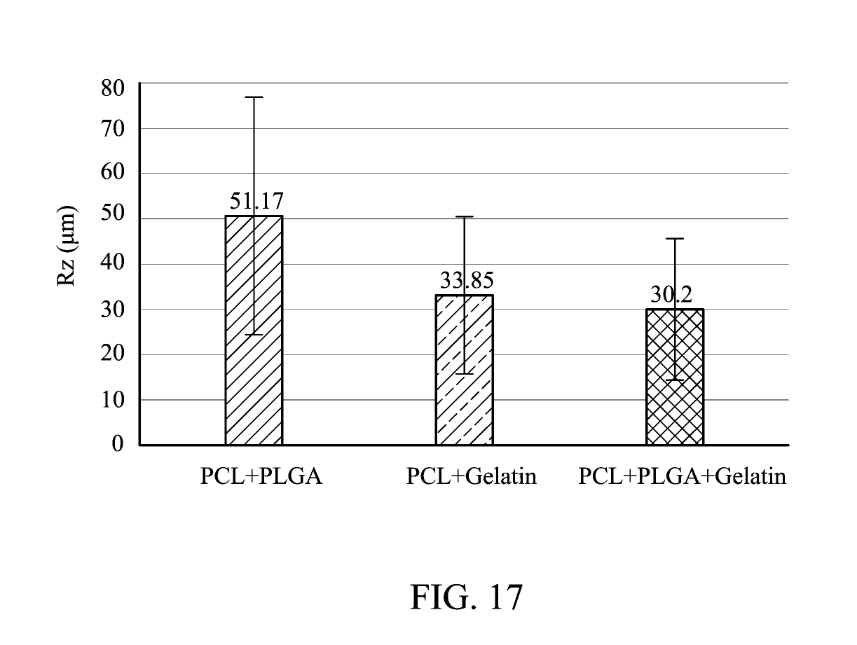

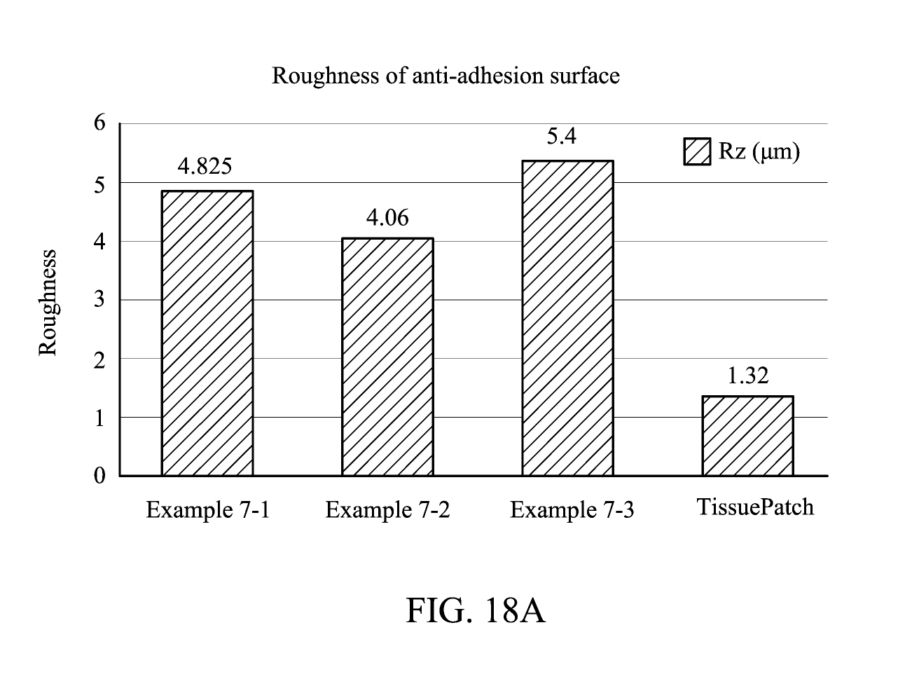

The results are shown in FIG. 17. Generally, in a condition without using a surfactant, a hydrophilic polymer and a hydrophobic polymer are not compatible, and that results in microphase separation and makes the film surface rough and uneven.

As shown in FIG. 17, the film prepared in Comparative Example 1 without using the process of the present disclosure (the film prepared in Comparative Example 1 (polycaprolactone/poly(lactic-co-glycolic acid))) still suffered from microphase separation due to the two polymers having different properties, and that resulted in surface microphase separation and a rough structure. In contrast, for the film prepared by the process in which gelatin was used as a dispersing agent of the present disclosure (the film prepared in Example 3-1 (polycaprolactone/poly(lactic-co-glycolic acid)/gelatin)), the gelatin in the film would not influence the roughness of the film surface, and no phase separation appeared in the hydrophilic polymer and the hydrophobic polymer in the film, giving the film surface a smooth and compact structure.

(2-2) Roughness Analysis for the Two Side of the Double Layer Films Prepared in Examples 7-1 to 7-5

Roughness analysis was performed on the two sides of the films prepared in Examples 7-1 to 7-5 and a sealing patch in clinical use (TissuePatch). The operation procedure is the same as the operation procedure described in (2-1) above. Moreover, the films prepared in Examples 7-1 to 7-3 and the sealing patch in clinical use (TissuePatch) were tested in one batch of experiments while the films prepared in Example 7-1, Example 7-4 and Example 7-5 and the sealing patch in clinical use (TissuePatch) were tested in another batch of experiments.

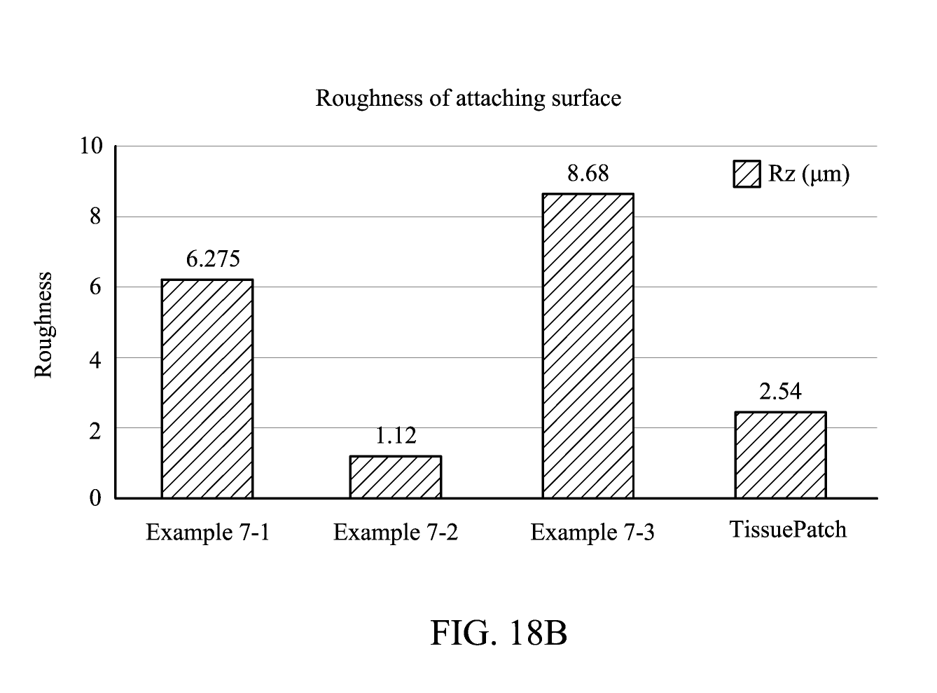

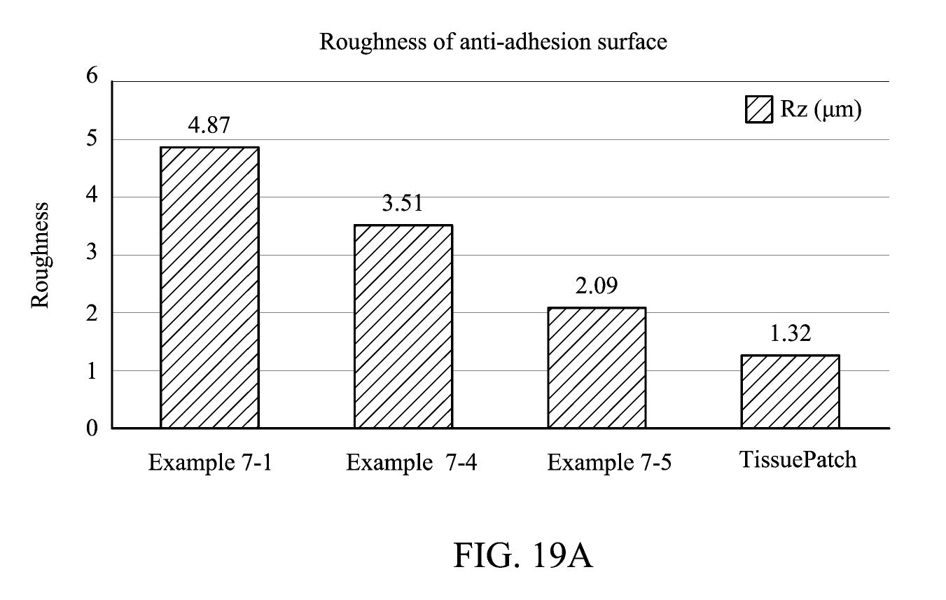

The analysis results of anti-adhesion surfaces and the attaching surfaces of the films prepared in Examples 7-1 to 7-3 and sealing patch in clinical use (TissuePatch) in one batch of experiments are shown in FIGS. 18A and 18B, respectively.

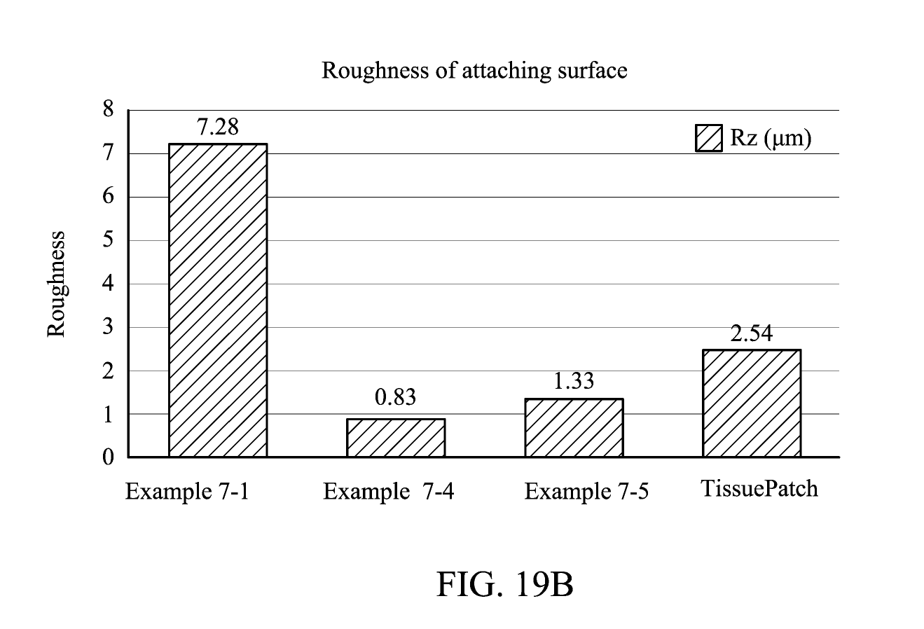

The analysis results of anti-adhesion surfaces and the attaching surfaces of the films prepared in Example 7-1, Example 7-4 and Example 7-5 and sealing patch in clinical use (TissuePatch) in the other batch of experiments are shown in FIGS. 19A and 19B, respectively.

FIGS. 18A and 18B and FIGS. 19A and 19B show that the roughness of the anti-adhesion surfaces of the films prepared in Examples 7-1 to 7-5 are all greater than that of sealing patch in clinical use (TissuePatch).

(3) Thickness Analysis

Thickness analysis is performed on Examples 7-1 to 7-5 and sealing patch in clinical use (TissuePatch)

Operation procedure of thickness analysis is summarized in the following.

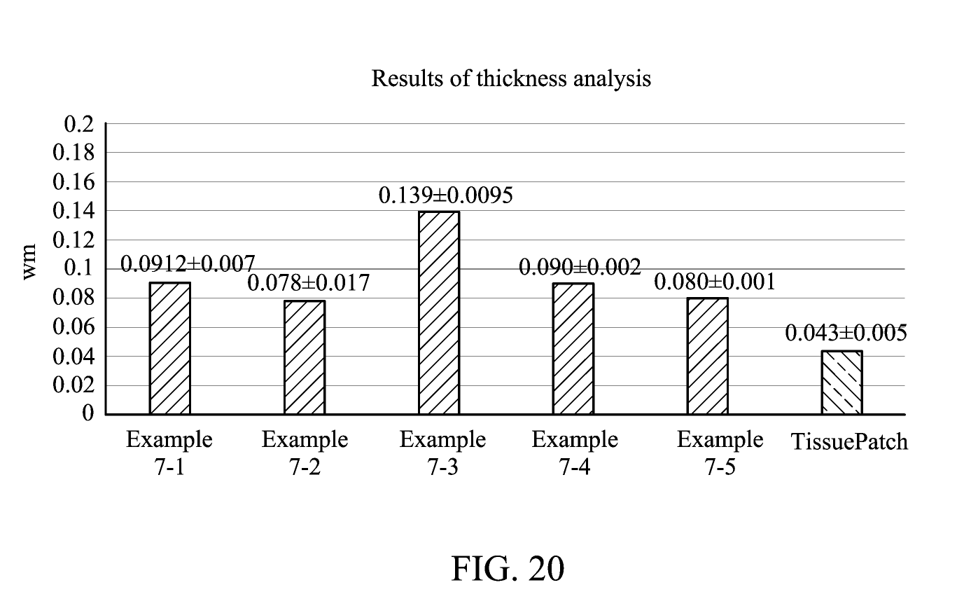

A depth gauge was used to perform the analysis. First, a sample to be tested was evenly attached to a marble stage and fixed to prevent occurrence of irregular or winding condition. Next, the probe of the depth gauge after being reset to zero was contacted with the surface of the film to let the film locate between the marble and the probe to perform the determination of thickness and record. The results are shown in FIG. 20

FIG. 20 shows that the thickness the films prepared in Examples 7-1 to 7-5 are all greater than that of sealing patch in clinical use (TissuePatch).

4. Animal Experiments

(1) Attachment Experiment for Rat Liver

First, Rompun 20 and Zoletil 50 were mixed at a ratio of 1:1 to formulate an anesthetic. Anesthesia was administered to a rat with 0.4 ml/kg of anesthetic by intramuscular injection (IM) at the thigh muscles.

After the rat was deeply anaesthetized, hairs of the rat on the location to be subjected to a surgery were sheared. After that, the rat was placed on an aseptic operating table and covered with an aseptic hole-towel, and the location to be subjected to surgery was surface sterilized with povidone iodine.

The abdomen of the rat was cut to open and the position of the liver was found, and then a surface wound was made on the liver, and the size of the wound was about 1-1.5 CM.

Next, a film of the present disclosure was attached to the wound, forming a patch. After the attachment, the wound was allowed to stand for 30 seconds to confirm that the film was indeed attached to the wound. After that, abdominal muscles and epidermis of the rat were sutured. After suturing, the sutured positions were surface sterilized with povidone iodine to complete the surgery.

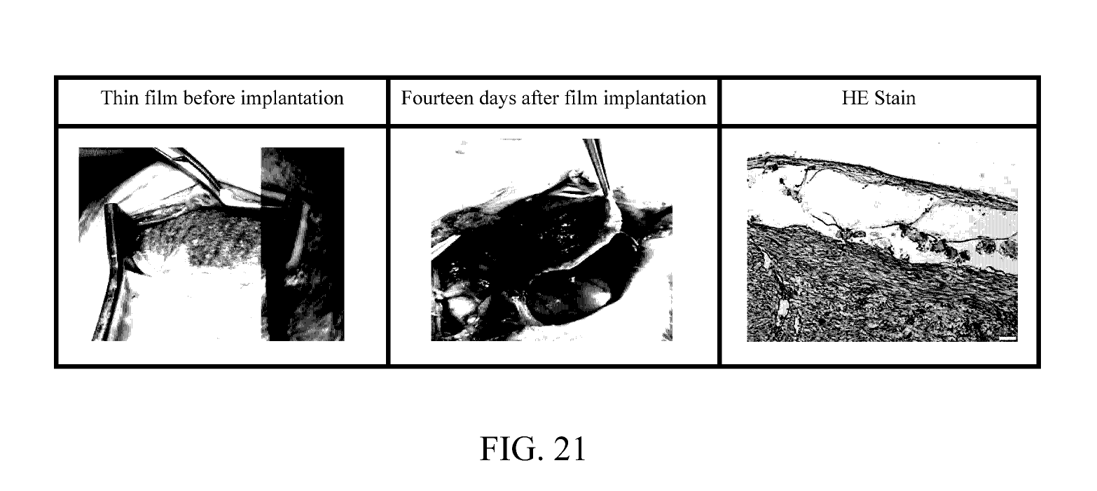

After the surgery, physiological observation was performed on the animal. 14 days after the film was implanted, the rat was sacrificed. After that, the appearance of the surgical site was observed and photographed, and the tissue to which the film was attached was sampled and a hematoxylin and eosin (H&E) stain was performed thereon. The results are shown in FIG. 21.

The results showed that 14 days after the surgery, the film was still capable of effectively attaching to the original surgical site.

(2) Animal Attachment Experiment for Rat Stomach

First, Rompun 20 and Zoletil 50 were mixed at a ratio of 1:1 to formulate an anesthetic. Anesthesia was administered to a rat with 0.4 ml/kg of anesthetic by intramuscular injection (IM) at the thigh muscles.

After the rat was deeply anaesthetized, hairs of the rat on the location to be subjected to a surgery were sheared. After that, the rat was placed on an aseptic operating table and covered with an aseptic hole-towel, and the location to be subjected to surgery was surface sterilized with povidone iodine.

A laparotomy was performed on the rat, and the stomach was cut to make a perforation with a length of about 0.5 cm, and then in the middle of the perforation, i.e. in a position about 0.25 cm along the perforation length, a stitch was made using sutures to create a leaking wound. After that, a film of the present disclosure was attached directly to the leaking wound.

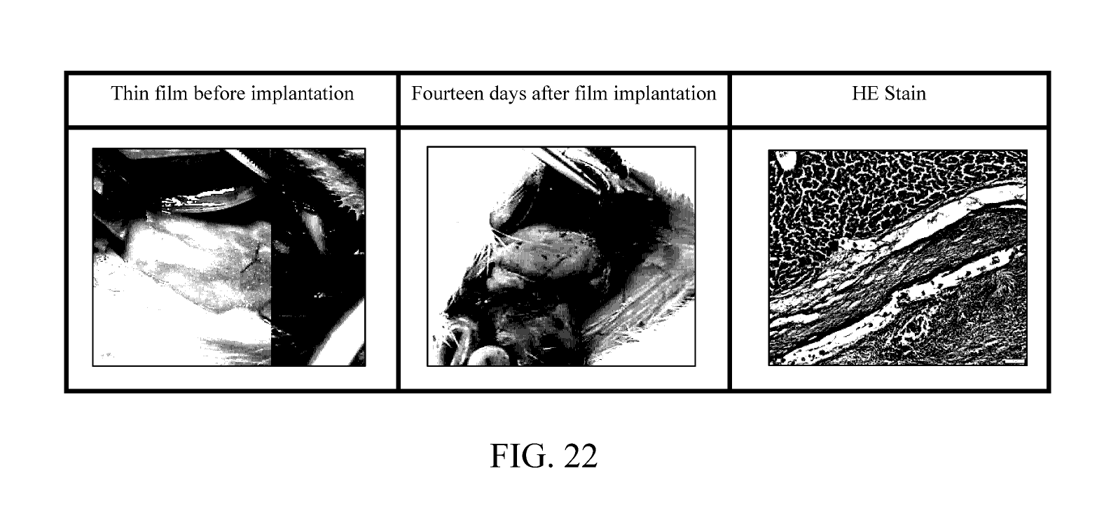

After the surgery, physiological observation was performed on the animal. 14 days after implanting the film, the rat was sacrificed. After that, the appearance of the surgical site was observed and photographed, and the tissue to which the film was attached was sampled and a hematoxylin and eosin (H&E) stain was performed thereon. The results are shown in FIG. 22.

According to FIG. 22, only through visual observation could it be confirmed that the film of the present disclosure was still at the original surgical site.

The results of the film patching experiment on the two animal organs showed that, after being attached to the liver and stomach, the films were not fixed with sutures.

However, 2 weeks after implanting the films of the present disclosure to a living body, it was confirmed through observation that the films of the present disclosure were still at the original surgical sites (see FIG. 21 and FIG. 22).

In addition, the results of the hematoxylin and eosin (H&E) stains also showed that the film would not induce a serious immune response and could promote tissue repair.

Attachment Experiment for Rat Intestine

First, Rompun 20 and Zoletil 50 were mixed at a ratio of 1:1 to formulate an anesthetic. Anesthesia was administered to a rat with 0.4 ml/kg of anesthetic by intramuscular injection (IM) at the thigh muscles. After a wait of 5 minutes, the reflex action of the rat was checked to determine whether the rat was under anesthetic, for example by observing its breathing or providing a pain test to the periphery.

After the rats were anesthetized, shearing and sterilization were performed on the location of the rats to be subjected to surgery. The position located at a position 1 cm under the xiphoid and then 0.5 cm right shifted side (according to the physiological structure of rats) was set as the location to be subjected to a surgery.

After the aforementioned sterilization step was completed, before the surgery, 0.5 ml of lidocaine was subcutaneously injected in the location to be subjected to a surgery to perform local anesthesia. The epidermis of the location to be subjected to a surgery was incised in a longitudinal direction by a scalpel. Next, after the subcutaneous muscle layer was cut in the same direction with a tissue scissor, the location of the liver and intestine can be seen. The location was orientated as a surgery location for intestine, wherein the length of the surgery location was about 2 cm while the right side and the left side of the surgery location were orientated by 4-0 nylon suture.

After that, about 30 perforations were created on the intestine by a 18G injection needle at a surgery location in range of about 2 cm to injure the tissue and result in leakage to ensure inflammation of the surgery location and damage to the intestine, to cause the occurrence of the adhesion between the intestine and other tissues.

After establishment of the damage to the intestine tissue, subcutaneous and epidermal sutures were directly performed on the rats of the control group, while for the rats of the experimental group, film implantation was performed, wherein the anti-adhesion films prepared in Example 7-1, Example 7-5 and Example 7-6 and the sealing patch in clinical use (TissuePatch) were implanted into the surgery locations and attached the wounds by the attaching surfaces thereof. Then, the wound was wound with a film for one and a half rounds so that the wound was covered by the attaching surface of the film. After the film implantation was completed, the abdominal muscle layer and subcutaneous layer of the rat was sutured and sterilized, and the film was implanted for 1 month.

One month later, the animal's appearance was observed and the animals were sacrificed and dissected to observe the appearance of the tissue and evaluate the anti-adhesion properties of the film.

Epidermal tissue (including muscular layer) dissection was performed in a concave-shaped manner after the rat was sacrificed. After dissection, the intraperitoneal tissue and the environment inside the body were observed, and the surgery location was confirmed and photographed to evaluate the adhesion condition of the surgery location.

The adhesion evaluation manner is based on the adhesion quantification score table recited in the literature Hernia 14 (6): 599-610, December 2010.

If adhesion of intestinal tissue of the surgery locations to other tissue did not occur and the surfaces of intestinal tissue of the surgery locations were smooth and even without occurrence of other fibrous tissue, a score of 1 was assigned. If adhesion of the surgery locations to other tissue occurred and during the sampling process the adhesion can be easily separated from other tissue without the help of an external force or instrumental separation, it represented that the adhesion condition was slight, and a score of 2 was assigned. If in the sampling process, the adhesion occurred at the surgery locations, and the sampling process required the assistance of tools or an external force to separate the adhesion tissues, a score of 3 was assigned to the adhesion condition. If in the sampling process, adhesion occurred and the adhesion tissues could not be separated by an external force or tools and needed to be separated by cutting or other truncated way, it was the most serious state of adhesion and a score of 4 was assigned.

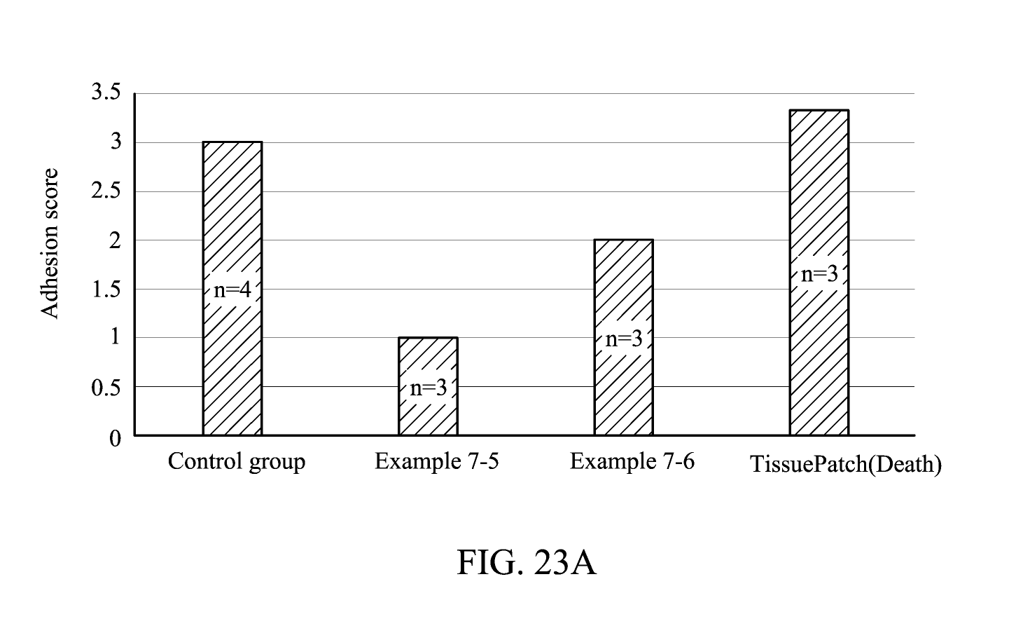

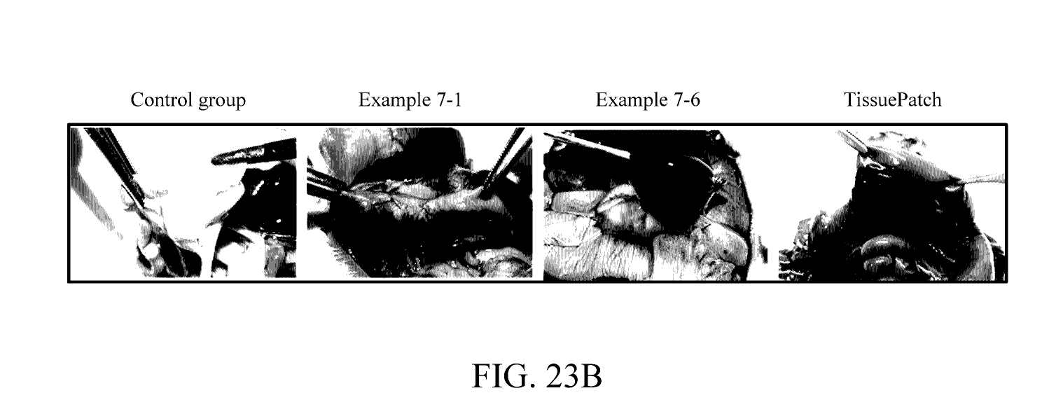

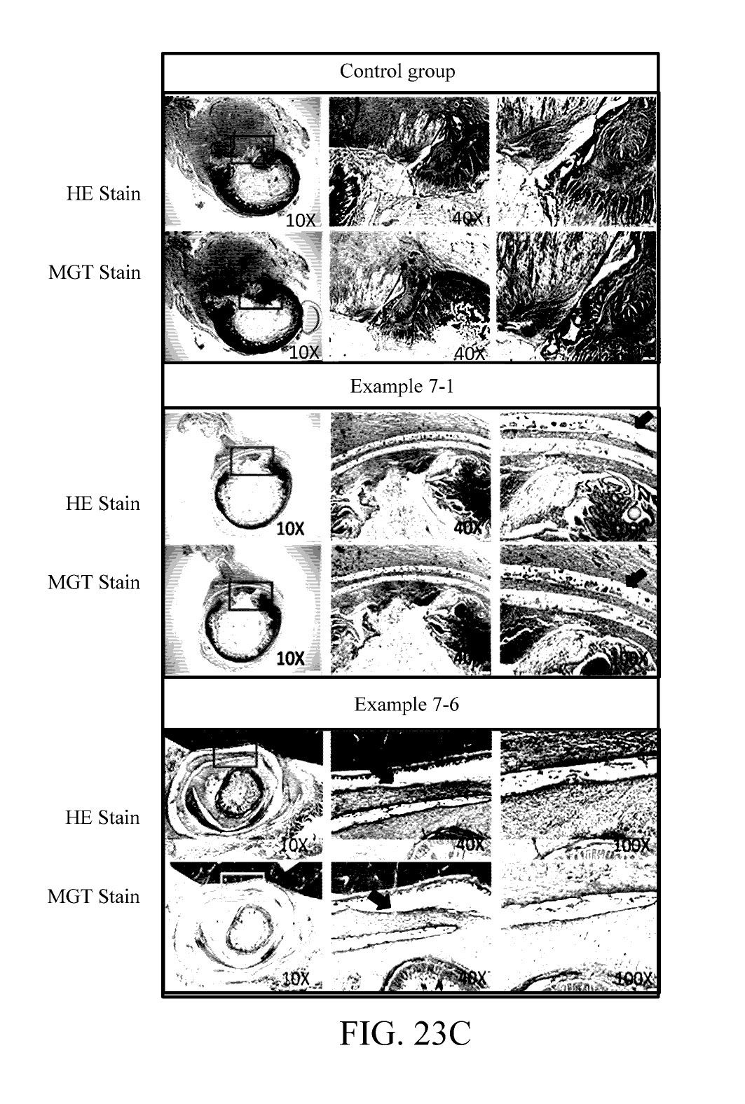

The results are shown in FIGS. 23A and 23B.

After completion of sacrifice and evaluation of the appearance and tissue adhesion, the intestine of the surgery locations was sampled and washed with saline.

After the cleaning step was completed, the intestine sample was fixed with 10% of formalin, and the fixing time was 16-24 hours.

Then, hematoxylin and eosin (H&E) stain and modified Gomori Trichrome (MGT) stain were performed on the intestine samples. The results are shown in FIG. 23C. The positions indicated by the arrows are the positions of the films. Refer to the positions indicated by the arrows in the 100× photograph of Example 7-1 in FIG. 23C, the anti-adhesion adhesive film of the present disclosure can effectively cover the tissue defect location. 40× and 100× photographs are the boxes in the 10× photograph that have been magnified.

According to FIGS. 23A, 23B and 23C, it is known that both the control group and the TissuePatch cause adhesion, and the TissuePatch even causes the death of the rat, whereas the double layer films of the present disclosure have excellent anti-adhesion effects.

It will be apparent to those skilled in the art that various modifications and variations can be made to the disclosed embodiments. It is intended that the specification and examples be considered as exemplary only, with the true scope of the disclosure being indicated by the following claims and their equivalents.

1. A film, composed of a polymer mixture,
wherein the polymer mixture comprises:
a hydrophobic composition comprising polycaprolactone (PCL); and
at least one hydrophilic polymer, selected from a group consisting of:
alginate, gelatin, hyaluronic acid, polyvinyl alcohol (PVA), carboxymethyl cellulose (CMC), polyethylene glycol (PEG), collagen, demineralized bone matrix (DBM), bone morphogenetic protein (BMP), albumin, chitosan, fibrin, polyoxyethylene and polyvinylpyrrolidone,
wherein a weight ratio of the hydrophobic composition to the at least one hydrophilic polymer is about 1:0.01-100.

2. The film as claimed in claim 1, wherein the at least one hydrophilic polymer is in the form of a solid particle.

3. The film as claimed in claim 1, wherein the at least one hydrophilic polymer is dissolved in a solvent to be in the form of a liquid polymer.

4. The film as claimed in claim 1, wherein the at least one hydrophilic polymer is alginate.

5. The film as claimed in claim 4, wherein the weight ratio of the hydrophobic composition to the alginate is about 1:0.05-80.

6. The film as claimed in claim 1, wherein the at least one hydrophilic polymer is gelatin.

7. The film as claimed in claim 6, wherein the weight ratio of the hydrophobic composition to the gelatin is about 1:0.05-80.

8. The film as claimed in claim 1, wherein the hydrophobic composition further comprises at least one hydrophobic polymer which is selected from a group consisting of polylactic acid (PLA), poly(lactic-co-glycolic acid) (PLGA), poly(glycolic acid) (PGA), polyhydroxybutyrate (PHB), polydioxanone (PDS), poly(propylene fumarate) (PPF), polyanhydrides, polyacetals, poly(ortho esters), polycarbonates, polyurethanes, polyphosphazenes and polyphosphoester.

9. The film as claimed in claim 8, wherein the at least one hydrophobic polymer is poly(lactic-co-glycolic acid).

10. The film as claimed in claim 9, wherein the at least one hydrophilic polymer is alginate.

11. The film as claimed in claim 10, wherein the weight ratio of the hydrophobic composition to the alginate is about 1:0.05-80.

12. The film as claimed in claim 9, wherein the at least one hydrophilic polymer is gelatin.

13. The film as claimed in claim 12, wherein the weight ratio of the hydrophobic composition to the gelatin is about 1:0.05-80.

14. A method for manufacturing a film, comprising:
preparing a polymer mixture, wherein a method for preparing the polymer mixture comprises:
preparing a hydrophobic solution, wherein a solute of the hydrophobic solution comprises polycaprolactone; and
adding at least one hydrophilic polymer as a dispersing agent to the hydrophobic solution and mixing it with the hydrophobic solution, wherein the at least one hydrophilic polymer is selected from a group consisting of: alginate, gelatin, hyaluronic acid, polyvinyl alcohol (PVA), carboxymethyl cellulose (CMC), polyethylene glycol (PEG), collagen, demineralized bone matrix (DBM), bone morphogenetic protein (BMP), albumin, chitosan, fibrin, polyoxyethylene and polyvinylpyrrolidone,
wherein the weight ratio of the solute of the hydrophobic solution to the at least one hydrophilic polymer is about 1:0.01-100; and
drying the polymer mixture to form a film.

15. The method for manufacturing a film as claimed in claim 14, wherein the at least one hydrophilic polymer is in the form of a solid particle.

16. The method for manufacturing a film as claimed in claim 14, wherein the at least one hydrophilic polymer is dissolved in a solvent to be in the form of a liquid polymer.

17. The method for manufacturing a film as claimed in claim 14, wherein the viscosity of the polymer mixture is about 300-700 CP.

18. The method for manufacturing a film as claimed in claim 14, wherein the at least one hydrophilic polymer is alginate.

19. The method for manufacturing a film as claimed in claim 18, wherein the weight ratio of the polycaprolactone to the alginate is about 1:0.05-80.

20. The method for manufacturing a film as claimed in claim 14, wherein the at least one hydrophilic polymer is gelatin.

21. The method for manufacturing a film as claimed in claim 20, wherein the weight ratio of the polycaprolactone to the gelatin is about 1:0.05-80.

22. The method for manufacturing a film as claimed in claim 14, wherein the solute of the hydrophobic solution further comprises a hydrophobic polymer and the hydrophobic polymer is selected from a group consisting of polylactic acid (PLA), poly(lactic-co-glycolic acid) (PLGA), poly(glycolic acid) (PGA), polyhydroxybutyrate (PHB), polydioxanone (PDS), polypropylene fumarate) (PPF), polyanhydrides, polyacetals, poly(ortho esters), polycarbonates, polyurethanes, polyphosphazenes and polyphosphoester.

23. The method for manufacturing a film as claimed in claim 22, wherein the at least one hydrophobic polymer is poly(lactic-co-glycolic acid).

24. The method for manufacturing a film as claimed in claim 23, wherein the at least one hydrophilic polymer is alginate.

25. The method for manufacturing a film as claimed in claim 24, wherein the weight ratio of the solute of the hydrophobic solution to the alginate is about 1:0.05-80.

26. The method for manufacturing a film as claimed in claim 23, wherein the at least one hydrophilic polymer is gelatin.

27. The method for manufacturing a film as claimed in claim 26, wherein the weight ratio of the solute of the hydrophobic solution to the gelatin is about 1:0.05-80.

28. A double layer film, comprising:
an attachment layer; and
an anti-adhesion layer on a surface of the attachment layer and bonded thereto,
wherein the attachment layer and the anti-adhesion layer are each independently a film as claimed in claim 1,
wherein, in the attachment layer, the at least one hydrophilic polymer is selected from a group consisting of: alginate, gelatin, collagen, demineralized bone matrix, bone morphogenetic protein, albumin, chitosan, fibrin, polyoxyethylene and polyvinylpyrrolidone, and
wherein, in the anti-adhesion layer, the at least one hydrophilic polymer is selected from a group consisting of: hyaluronic acid, polyvinyl alcohol, carboxymethyl cellulose and polyethylene glycol.

29. The double layer film as claimed in claim 28, wherein in the double layer film, the weight ratio of the total amount of the hydrophobic composition to the total amount of the hydrophilic polymer is about 1:0.1-10.

30. The double layer film as claimed in claim 28, wherein in the attachment layer, the content of the at least one hydrophilic polymer is about 10-80 wt %.

31. The double layer film as claimed in claim 28, wherein in the anti-adhesion layer, the content of the at least one hydrophilic polymer is about 0.1-30 wt %.

32. The double layer film as claimed in claim 28, wherein in the attachment layer, the at least one hydrophilic polymer is gelatin.

33. The double layer film as claimed in claim 28, wherein in the anti-adhesion layer, the at least one hydrophilic polymer is hyaluronic acid.

34. The double layer film as claimed in claim 28, wherein in the anti-adhesion layer, the at least one hydrophilic polymer is a combination of hyaluronic acid and polyvinyl alcohol.

35. The double layer film as claimed in claim 34, wherein in the anti-adhesion layer, the weight ratio of the hyaluronic acid to the polyvinyl alcohol is about 1:0.5-5.

36. The double layer film as claimed in claim 28, wherein in the anti-adhesion layer, the at least one hydrophilic polymer is a combination of carboxymethyl cellulose and polyethylene glycol.

37. The double layer film as claimed in claim 36, wherein in the anti-adhesion layer, the weight ratio of the carboxymethyl cellulose to the polyethylene glycol is about 1:0.1-30.

38. A method for manufacturing a double layer film, comprising:
(a) preparing a first polymer mixture and a second polymer mixture,
wherein a method for preparing the first polymer mixture comprises:
preparing a first hydrophobic solution, wherein a solute of the first hydrophobic solution comprises polycaprolactone; and
adding at least one hydrophilic polymer as a first dispersing agent to the first hydrophobic solution and mixing it with the first hydrophobic solution to form the first polymer mixture, wherein the at least one hydrophilic polymer is selected from a group consisting of: alginate, gelatin, collagen, demineralized bone matrix, bone morphogenetic protein, albumin, chitosan, fibrin, polyoxyethylene and polyvinylpyrrolidone,
wherein the amount of first dispersing agent added is sufficient to let the first polymer mixture become a homogeneous mixture in colloidal form,
wherein a method for preparing the second polymer mixture comprises:
preparing a second hydrophobic solution, wherein a solute of the second hydrophobic solution comprises polycaprolactone; and
adding at least one hydrophilic polymer as a second dispersing agent to the second hydrophobic solution and mixing it with the second hydrophobic solution to form the second polymer mixture, wherein the at least one hydrophilic polymer is selected from a group consisting of: hyaluronic acid, polyvinyl alcohol, carboxymethyl cellulose, and polyethylene glycol,
wherein the amount of second dispersing agent added is sufficient to let the second polymer mixture become a homogeneous mixture in colloidal form,
(b) drying the first polymer mixture to form a film to form an attachment layer; and
(c) drying the second polymer mixture on the attachment layer to form a film to form an anti-adhesion layer to complete the manufacture of the double layer film,
wherein the solvent of the first hydrophobic solution and the solvent of the second hydrophobic solution are the same.

39. The method for manufacturing a double layer film as claimed in claim 38, wherein in the first polymer mixture, the solid content is about 10-60 wt % and a weight ratio of the solute of the first hydrophobic solution to the first dispersing agent is about 1:0.1-5, and in the second polymer mixture, the solid content is about 0.1-30 wt % and a weight ratio of the solute of the second hydrophobic solution to the second dispersing agent is about 1:0.01-10.

40. The method for manufacturing a double layer film as claimed in claim 38, wherein the viscosity of the first polymer mixture is about 300-700 CP, and the viscosity of the second polymer mixture is about 300-700 CP.

41. The method for manufacturing a double layer film as claimed in claim 38, wherein in the attachment layer, the at least one hydrophilic polymer is gelatin.

42. The method for manufacturing a double layer film as claimed in claim 38, wherein in the anti-adhesion layer, the at least one hydrophilic polymer is hyaluronic acid.

43. The method for manufacturing a double layer film as claimed in claim 38, wherein in the anti-adhesion layer, the at least one hydrophilic polymer is a combination of hyaluronic acid and polyvinyl alcohol.

44. The method for manufacturing a double layer film as claimed in claim 43, wherein in the anti-adhesion layer, the weight ratio of the hyaluronic acid to the polyvinyl alcohol is about 1:0.5-5.

45. The method for manufacturing a double layer film as claimed in claim 38, wherein in the anti-adhesion layer, the at least one hydrophilic polymer is a combination of carboxymethyl cellulose and polyethylene glycol.

46. The method for manufacturing a double layer film as claimed in claim 45, wherein in the anti-adhesion layer, the weight ratio of the carboxymethyl cellulose to the polyethylene glycol is about 1:0.1-30.

47. The method for manufacturing a double layer film as claimed in claim 38, wherein the solvent of the first hydrophobic solution and the solvent of the second hydrophobic solution comprise acetone, acetic acid, chloroform, methanol, dichloromethane, dimethylformamide, dioxane, ethyl acetate, formic acid, hexafluoroisopropanol, 1-methyl-2-pyrrolidone, tetrahydrofuran, toluene, or a mixture solution thereof.

48. A method for sealing a surgical wound or a diffuse wound, comprising:
attaching the film as claimed in claim 1 to a surgical wound or a diffuse wound of a subject to seal the surgical wound or the diffuse wound.

49. The method for sealing a surgical wound or a diffuse wound as claimed in claim 48, wherein the hydrophobic composition of the film further comprises at least one hydrophobic polymer which is selected from a group consisting of polylactic acid (PLA), poly(lactic-co-glycolic acid) (PLGA), poly(glycolic acid) (PGA), polyhydroxybutyrate (PHB), polydioxanone (PDS), poly(propylene fumarate) (PPF), polyanhydrides, polyacetals, poly(ortho esters), polycarbonates, polyurethanes, polyphosphazenes, and polyphosphoester.
